(19) United States
(12) Patent Application Publication
(10) Pub. No.: US 2024/0166723 A1
(43) Pub. Date: May 23, 2024

(54) MECHANOCHEMICAL GENERATION OF NOVEL COLLAGEN GEL ARCHITECTURES AND SCAFFOLDS

(71) Applicant: Yale University, New Haven, CT (US)

(72) Inventors: Michael Mak, New Haven, CT (US); Chang Liu, New Haven, CT (US); Ryan Nguyen, New Haven, CT (US)

(21) Appl. No.: 18/503,544

(22) Filed: Nov. 7, 2023

Related U.S. Application Data
(60) Provisional application No. 63/481,511, filed on Jan. 25, 2023.
(60) Provisional application No. 63/382,571, filed on Nov. 7, 2022.

(51) Int. Cl. C07K 14/78; C07K 1/113

(52) U.S. Cl. CPC C07K 14/78; C07K 1/113

(57) ABSTRACT

A method of generating thickened collagen bundles or clusters, comprising the steps of: preparing a neutralized collagen gel sample; warming the neutralized collagen gel sample to provide a warmed neutralized collagen gel sample; and agitating the warmed neutralized collagen gel sample to provide an agitated collagen gel sample.

### CROSS REFERENCE TO RELATED APPLICATIONS

This application claims priority to and the benefit of U.S. Provisional Application No. 63/481,511, filed Jan. 25, 2023, and U.S. Provisional Application No. 63/382,571, filed Nov. 7, 2022, the disclosures of which are incorporated herein by reference herewith in their entireties.

### STATEMENT REGARDING FEDERALLY SPONSORED RESEARCH OR DEVELOPMENT

This invention was made with government support under R35GM142875 awarded by the National Institutes of Health (NIH). The government has certain rights in the invention.

### BACKGROUND OF THE INVENTION

Tumors are often described as “wounds that do not heal” (Dvorak, The New England Journal of Medicine, 1986, 315) since tumorigenesis is tightly associated with chronic tissue damage and repair processes (Arwert et al., Nature Reviews Cancer, 2012, 12). The hallmarks of cancer highly overlap with those of wound healing (MacCarthy-Morrogh, Science Signaling 2020, 13, eaay8690), both featuring sustained proliferative signaling, cell migration, angiogenesis, immune activation and inflammation and tissue repair with extracellular matrix (ECM) remodeling (MacCarthy-Morrogh, Science Signaling, 2020, 13, eaay8690).

In scar tissue formation, after initial blood clots are formed, there is a rapid recruitment of neutrophils and monocytes, with the onset of inflammation. Meanwhile, activation of myofibroblasts induces wounded tissue contraction and increased ECM deposition and remodeling, followed by re-epithelialization and angiogenesis to restore tissue homeostasis (Xue & Jackson, Advances in Wound Care: The Journal For Prevention And Healing, 2015, 4). However, cancer cells harbor high genomic instability and resistance to cell death, which fundamentally differentiate tumor progression from normal wound healing (MacCarthy-Morrogh, Science Signaling, 2020, 13, eaay8690; Behan et al., Nature, 2019, 568). Solid tumors and fibrotic diseases include most of these processes, but in a highly dysregulated and tissue dependent manner. Importantly, pathological ECM remodeling (Cox & Erler, Disease Models & Mechanisms, 2011, 4; Yuzhalin et al., Biochimica Et Biophysica Acta, Reviews On Cancer, 2018, 1870) is usually implicated in liver carcinoma (Nguyen et al., Communications Biology, 2022, 5, 202), breast cancer (Conklin et al., The American Journal Of Pathology, 2011, 178, 1221), melanoma (Kaur et al., Cancer Discovery, 2019, 9, 64) and lung cancer (Wood et al., Cancer Treatment Reviews, 2014, 40, 558). The dysregulated ECM signatures are directly associated with poor prognosis and immunotherapy failure (Chakravarthy et al., Nature Communications, 2018, 9, 4692).

3D in vitro models have been used to mimic specific aspects of the tumor microenvironment (TME) and study the dynamic ECM evolution (Pizzurro et al., Cancers, 2021, 13). Previous work on reconstructing the tumor ECM in vitro has primarily focused on individual local parameters, such as pore size, fiber diameter (Seo et al., Proceedings Of The National Academy Of Sciences Of The United States Of America, 2020, 117, 11387; Kalbitzer & Pompe, Acta Biomaterialia, 2018, 67, 206; Sapudom et al., Biomaterials, 2015, 52, 367; Yang et al., Biomaterials, 2010, 31, 5678), stiffness (Mason et al., Acta Biomaterialia, 2013, 9, 4635), viscoelasticity (Wisdom et al., Nature Communications, 2018, 9, 4144), collagen alignment (Riching et al., Biophysical Journal, 2014, 107, 2546; Mosier et al., Biophysical Journal, 2019, 117, 1692) or microarchitecture (Fischer et al., Proceedings Of The National Academy Of Sciences Of The United States Of America, 2021, 118; Sakar et al., Nature Communications, 2016, 7, 11036; Guzman et al., Biomaterials 2014, 35, 6954). However, these models usually only include sub-micrometer topographies and mechanics but lack global architectural similitude to in vivo ECM structure.

Thus, there is a need in the art for a novel method of generating thickened collagen bundles and clusters. The present invention satisfies this unmet need.

### SUMMARY OF INVENTION

In one aspect, the present invention relates to a method of generating thickened collagen bundles or clusters, comprising the steps of: preparing a neutralized collagen gel sample; warming the neutralized collagen gel sample to provide a warmed neutralized collagen gel sample; and agitating the warmed neutralized collagen gel sample to provide an agitated collagen gel sample.

In one embodiment, the method further comprises the steps of: cooling the agitated collagen gel sample to about 0° C. for about 10 to 30 minutes to provide a cooled collagen gel sample; and reagitating the cooled collagen gel sample at a temperature greater than or equal to 0° C. In one embodiment, the method further comprises the step of allowing the neutralized collagen gel sample to sit at room temperature for 6 to 10 minutes. In one embodiment, the method further comprises the step of cooling the neutralized collagen gel sample to 0° C.

In one embodiment, the step of warming the neutralized collagen gel sample comprises the step of heating the neutralized collagen gel sample to a temperature greater than or equal to 20° C. In one embodiment, the step of agitating the warmed neutralized collagen gel sample comprises the step of mixing the warmed neutralized collagen gel sample with a pipette.

In one embodiment, the agitated collagen gel sample has a turbidity which measures above background. In one embodiment, the step of agitating the warmed neutralized gel sample is automated via a mixing device.

In one embodiment, the step of agitating the warmed neutralized collagen gel is performed for over about 10 minutes. In one embodiment, the step of agitating the warmed neutralized collagen gel provides disconnected collagen clusters.

In one embodiment, the method further comprises the step of mixing the disconnected collagen clusters with cells to provide a cell and collagen cluster mixture. In one embodiment, the cell and collagen cluster mixture self-aggregates. In one embodiment, the cell and collagen cluster mixture comprises a structure selected from the group consisting of compact cell-collagen clusters, spheroids, and fibrotic cell clusters.

In one embodiment, the neutralized collagen sample has a collagen concentration of about 1 mg/mL to about 10 mg/mL.

In one embodiment, the neutralized collagen sample comprises a collagen selected from the group consisting of type I collagen, type II collagen, type III collagen, type IV collagen, type XI collagen, thick collagen, hydrolyzed collagen, mammal collagen, powdered collagen, and synthetic collagen. In one aspect, the present invention provides a thickened collagen structure.

In one embodiment, the thickened collagen structure is macroporous or mesoporous. In one embodiment, the thickened collagen structure is used in a bioactive cue, an in situ injectable, a vaccine, a biomimetic material, or a vascular organoid. In one embodiment, the thickened collagen structure comprises at least one collagen island. In one embodiment, the area of the at least one collagen island is between 1 and 1500 μm².

### BRIEF DESCRIPTION OF THE DRAWINGS

The foregoing purposes and features, as well as other purposes and features, will become apparent with reference to the description and accompanying figures below, which are included to provide an understanding of the invention and constitute a part of the specification, in which like numerals represent like elements.

FIG. 1 comprises FIGS. 1A to 1E and depicts that thickened collagen bundles are curly and globally soft. FIG. 1A depicts structural differences of thick collagen bundles versus normal collagen. FIG. 1B depicts local (left) and global (right) view of thick collagen structure. MDA-MB-231 cells (life-act) seeded in thick collagen bundles respond to the local architectural heterogeneity, demonstrating an invasive phenotype. Thick collagen bundles vary in length and size but are holistically homogeneous in an overview tilescan imaging. Attached polystyrene beads indicate the existence of invisible network between thick collagen bundles. FIG. 1C depicts thick collagen (2 mg/ml) is globally soft compared with normal collagen (2 mg/ml). Unpaired t-test is performed. * indicates P<0.05, ** indicates P<0.01. FIG. 1D depicts simplified manual step-by-step protocol of creating long and thick collagen via collagen fibrillogenesis intervention. FIG. 1E depicts a customized homogenization device that can be used to automate the formation of thick collagen networks.

FIG. 2 comprises FIGS. 2A to 2F and depicts thickened collagen structure can be tuned by temperature and mixing. FIG. 2A depicts a schematic diagram demonstrates hypothetically how external disturbance in the collagen lateral growth stage triggers the formation of thick collagen bundles. FIG. 2B depicts that after thick collagen bundles are formed, ice incubation and remix before gelation (indicated in the dotted square) can tuned to modulate architecture of the thick collagen scaffold. FIG. 2C depicts that after thick collagen is formed, ice incubation decreases the overall granularity of the thick collagen system. More data can be seen in FIG. 9B. FIG. 2D depicts that reflectance imaging depicts that ice incubation between step 4 and step 5 (before complete gelation at 37° C.) reduces the size of collagen bundles. More data and binarized images can be seen in FIGS. 9C and 9D. See FIG. 2C for step 4 and step 5 description. FIG. 2E depicts that the frequency and strength applied in mixing thick collagen bundles before step 5 controls the size of collagen bundles. FIG. 2F depicts scanning electron microscope images of normal collagen versus thick collagen bundles. Normal collagen consist of dense network and small pores while thick collagen patches have long tortuous collagen bundles and less physical restraints. Red arrows indicate the boundary of a possible collagen patch.

FIG. 3 comprises FIGS. 3A to 3H and depicts MDA-MB-231s located on the boundary of thick collagen patch are more migratory. FIG. 3A depicts MDA-MB-231 (lifeact-GFP) cells seeded in normal type I collagen (Nor Col) versus thick collagen (Thick Col). MDA-MB-231 cells seeded in thickened collagen bundles do not form cell clusters and maintain an invasive phenotype throughout the culture period. Images from the top row are Z-projected images in which collagen structures are overlaid. Bottom row demonstrates one slice without projection. FIG. 3B depicts MDA-MB-231s (lifeact-GFP) seeded in thick collagen (Thick Col) bundles migrate more actively than normal collagen (Nor Col) on day0 and day1. FIG. 3C depicts average speed of MDA-MB-231s (lifeact-GFP) between normal versus thick collagen on day0 and day1. FIG. 3D depicts average mean squared displacement of MDA-MB-231s (lifeact-GFP) between normal versus thick collagen on day0 and day1. FIG. 3E depicts representative image demonstrates the distinct behavior of MDA-MB-231s (lifeact-GFP) along gel boundary versus trapped in the middle of the thick collagen. The beads trapped between the gel patches indicate the existence of loose connecting fiber but are invisible to reflectance microscopy. FIG. 3F depicts summarized morphology of MDA-MB-231s (lifeact-GFP) in different conditions. Thick-Boundary: on the boundary of thickened collagen patch; Thick-Inside: in the middle of thickened collagen patch; Normal: normal collagen. FIG. 3G depicts average speed of MDA-MB-231s (lifeact-GFP) located on the boundary versus cells found trapped in the middle of gel. FIG. 3H depicts circularity of MDA-MB-231s (lifeact-GFP) located on the boundary versus cells found trapped in the middle of gel. All data are collected from at least 3 independent experiments with at least 2 replicates in each experiment. One-way ANOVA with post-hoc Tukey HSD test was performed. * indicates p-values<0.05. ** indicates p-value<0.01.

FIG. 4 comprises FIGS. 4A to 4D and depicts drug treatment trigger different cell morphology patterns in thick collagen versus normal collagen. FIG. 4A depicts morphology of MDA-MB-231s (lifeact-GFP) under drug treatment on day2. FIG. 4B depicts summarized morphology of MDA-MB-231s (lifeact-GFP) under drug treatment on day2. SMIFH2 treated MDA-MB-231s do not demonstrate F-actin accumulation along cell boundaries (indicated by white arrow) compared with MDA-MB-231s under DMSO, GM6001, ML141, NSC23766, and EHT1864 treatment (indicated by red arrows). Red dotted circles demonstrate visual difference of MDA-MB-231 cell bodies in normal and thick collagen under Y27632 treatment. FIG. 4C depicts MDA-MB-231 (lifeact-GFP) population distribution when projected on the axis of elongation index, compactness index, shape variance, and sphericity, the definition and calculation of these four indexes in FIG. 11B. Red arrows indicate that the morphology response from the MDA-MB-231 population is similar under different drug treatments, which may suggest that there exists a specific subpopulation of MDA-MB-231s that respond most to collagen architecture (as indicated by yellow arrows). FIG. 4D depicts morphology index of each condition in normal (purple) and thick collagen (blue). All data are collected from at least 2 independent experiments with at least 2 replicates in each experiment. One-way ANOVA with post-hoc Tukey HSD test was performed for each collagen condition (thick or normal) separately. * indicates p-values<0.05. ** indicates p-value<0.01.“#” indicates that condition is significantly different from all other conditions on that day, unless otherwise annotated.

FIG. 5 comprises FIGS. 5A to 5C and depicts fast migration of MDA-MB-231s in thick collagen requires cell contractility and protrusions but not MMPs. FIG. 5A depicts overlaid trajectories of MDA-MB-231s (lifeact-GFP) in thick collagen versus normal collagen on day0 and day1. “#” indicates that condition is significantly different from all other conditions, unless otherwise annotated on that day. One-way ANOVA with Turkey Post hoc analysis was performed within each day of each collagen condition. * indicates P<0.05, ** indicates P<0.01. FIG. 5B depicts average speed of MDA-MB-231s (lifeact-GFP) in thick collagen versus normal collagen on day0 and day1. FIG. 5C depicts average mean squared displacement of MDA-MB-231s (lifeact-GFP) in thick collagen versus normal collagen day0 and day1. Error bar is in SEM. All data are collected from at least 2 independent experiments with at least 2 replicates in each experiment.

FIG. 6 comprises FIGS. 6A to 6H and depicts non-metastatic MCF10As acquire protrusive phenotype in thick collagen. FIG. 6A depicts projected confocal imaging of MCF10As in 2 mg/ml thick collagen, 1 mg/ml and 2 mg/ml normal collagen. FIG. 6B depicts compactness, elongation, sphericity, and shape variance index of MCF10As in different conditions. All data are collected from at least 2 repeated experiments with 2 replicates in each experiment. One-way ANOVA with Turkey Post hoc analysis was performed within each morphology index. * indicates P<0.05, ** indicates P<0.01. FIG. 6C depicts MCF10A cell population distribution is projected on the axis of elongation, compactness, shape variance, and sphericity. FIG. 6D depicts overlaid migration trajectories of MCF10As on day0 and day1. FIG. 6E depicts average speed of MCF10As on day0 and day1. One-way ANOVA with Turkey Post hoc analysis was performed within each day. * indicates P<0.05, ** indicates P<0.01. FIG. 6F depicts average mean squared displacement of MCF10As on day0 and day1. FIG. 6G depicts day4 old MCF10A acini in normal versus thick collagen on day7 (3 days after collagen embedding). Nor Col: Normal collagen. Thick Col: Thick collagen. Reflectance imaging is shown to demonstrate collagen thickening as a result of MCF10A acini contraction. FIG. 6H depicts MCF10A single cell seeded in normal collagen (Nor Col) developed into round or extended cell clusters on day3. MCF10As seeded in thick collagen (Thick Col) demonstrate an invasive phenotype, and almost no round cell clusters can be observed. In addition, the global contraction of thick collagen network happens.

FIG. 7 comprises FIGS. 7A to 7C and depicts molecular profiles induced by thick collagen in MCF10As. FIG. 7A depicts YAP/TAZ localization in MCF10A single cell and clusters on day 4. “MCF10A-N-Cell” indicates single MCF10As identified and measured in 2 mg/ml normal collagen. “MCF10A-T-Cell” indicates single MCF10As identified and measured in 2 mg/ml thick collagen. “MCF10A-N-Cluster” indicates MCF10A clusters (consisting of more than 2 nuclei) identified and measured in 2 mg/ml normal collagen. “MCF10A-T-Cluster” indicates MCF10A clusters (consisting of more than 2 nuclei) identified and measured in 2 mg/ml thick collagen. YAP/TAZ data are collected from 2 independent experiments with at least 3 replicates in each experiment. One-way ANOVA with Turkey Post hoc analysis was performed. * indicates P<0.05, ** indicates P<0.01. FIG. 7B depicts immunofluorescence staining of vinculin in normal versus thick collagen. The red arrow indicates the colocalization of vinculin and collagen pulling. FIG. 7C depicts pMLC staining in normal versus thick collagen. The red arrow indicates the colocalization of pMLC with the collagen pulling (alignment).

FIG. 8 depicts summarized features of thick collagen network compared with normal collagen. Thick collagen gels are heterogeneous and impose low physical restraints compared with normal collagen. Paratensile signaling may exist in thick collagen that allows for long range intercellular communication.

FIG. 9 comprises FIGS. 9A to 9E and depicts thickened collagen structure can be tuned by temperature and mixing. FIG. 9A depicts turbidimetry (A405) and storage modulus of thick collagen and normal collagen. Error bar is in SEM. FIG. 9B depicts additional photos which demonstrate the disassembly of thick collagen bundles before and after ice incubation. FIG. 9C depicts confocal reflectance imaging verified changes of collagen bundles before and after ice incubation. FIG. 9D depicts binarized and inverted images from FIG. 9C. FIG. 9E depicts in the top row representative images of thick collagen bundles produced manually from 5 independent cell seeding experiments with each image collected from one experiment. Variation can be seen in the produced collagen, but overall consistency can be acquired. The bottom row depicts 3 independent repeats demonstrate thick collagen bundles consistency fabricated from automatic mixing.

FIG. 10 comprises FIGS. 10A to 10D and depicts thick collagen highly mimics collagen structure from human skin scars. FIG. 10A depicts second-harmonic generation (SHG) images from human scars. (a-e) human scars of 2, 4, 8, 15, and 21 years age after wound. (f) Normal and healthy skin sample from 29 years old volunteer. FIG. 10B depicts second-harmonic generation (SHG) images of healthy human skin and skin of patient with Ehlers-Danlos syndrome. (a-b) Normal human skin. (c-f) Patient skin. The size of images is 150>150 μm. FIG. 10C depicts different structures can be produced by modulating collagen density. FIG. 10D depicts loose thick collagen network (in blue) shown in trichrome stained histological sample of day6 mouse melanoma (induced by YUMM injection). Cell nuclei are in black, cell cytoplasm and muscle are in red.

FIG. 11 comprises FIGS. 11A to 11C and depicts MDA-MB-231s located on the boundary of thick collagen patch are more invasive. FIG. 11A depicts the zoomed-in view of MDA-MB-231s (lifeact-GFP) on day5 in normal collagen(top) and thick collagen(bottom). FIG. 11B depicts left: Most MDA-MB-231s (lifeact-GFP) locate on the boundary of thick collagen patches on day 5. Right: the criterion used to differentiate middle vs boundary cancer cell. FIG. 11C depicts a histogram of average speed for FIG. 3C.

FIG. 12 comprises FIGS. 12A to 12D and depicts drug treatment triggers different cell morphology in thick collagen versus normal collagen. FIG. 12A depicts brightfield channel images in supplementary to FIG. 4A. FIG. 12B depicts an illustration of each morphology index used. FIG. 12C depicts normalized sphericity of MDA-MB-231(life-act) across all drug conditions. All drugs are normalized by sphericity in normal collagen+DMSO treatment condition. In DMSO/ML141/GM6001/EHT1864 treated conditions, normalized sphericity show similar decrease from normal collagen to thick collagen. Collagen architecture may dominate the morphological change instead of drug treatment. In Y27632/CK666/NSC23766/SMIFH2 treated condition, normalized sphericity is similar between normal collagen and thick collagen. Drug treatment instead of collagen architecture dominate morphological change. FIG. 12D depicts performance of imaging segmentation.

FIG. 13 comprises FIGS. 13A and 13B and depicts fast migration of MDA-MB-231 in thick collagen requires cell contractility and protrusions but not MMPs. FIG. 13A depicts histograms of average speed in supplementary of FIG. 5B. FIG. 13B depicts rearrangement of FIG. 5B with raw data overlaid.

FIG. 14 comprises FIGS. 14A to 14D and depicts non-metastatic MCF10As acquire invasive phenotype in thick collagen. FIG. 14A depicts average speed rearranged in supplementary to FIG. 6E. Unpaired t-test is performed. * indicates P<0.05. FIG. 14B depicts brightfield imaging of day 4 old MCF10A acini in normal versus thick collagen on day 7 (3 days after embedment in collagen). Supplementary to FIG. 6G. FIG. 14C depicts MCF10A single cells seeded in thick collagen demonstrate an invasive phenotype. Supplementary to FIG. 6D; The phenotypic change induced is universal and consistent in all replicates. FIG. 14D depicts a histogram of the average speed of MCF10A in supplementary to FIG. 6E.

FIG. 15 comprises FIGS. 15A to 15D and depicts molecular profiles induced by thick collagen in MCF10As. FIG. 15A depicts Yap/Taz localization in MCF10A single cell and clusters at day 4. Additional data in supplementary to FIG. 6A. FIG. 15B depicts a zoomed-out view of Yap/Taz localization in MCF10A single cell and clusters at day 4. Additional data in supplementary to FIG. 7A. FIG. 15C depicts immunofluorescence staining of vinculin in normal versus thick collagen. The red arrow indicates the colocalization of vinculin and collagen pulling—additional data in supplementary to FIG. 6B. FIG. 15D depicts phosphorylated-MLC staining in normal versus thick collagen. The red arrow indicates the colocalization of phosphorylated-MLC with the collagen pulling (alignment)—Additional data in supplementary to FIG. 7C.

FIG. 16 comprises FIGS. 16A to 16E and depicts thick collagen applied in MDA-MB-231 and NHLF coculture. FIG. 16A depicts the fast invasion of MDA-MB-231s (lifeact-GFP) in an overview tilescan imaging. The tilescan imaging tracks the expansion of each single cell colony along time. MDA-MB-231s (lifeact-GFP) fast “diffuse” along thick collagen bundles from 12 hrs post gel embedment to 36 hrs post gel embedment. FIG. 16B show both NHLF and MDA-MB-231(lifeact-GFP) spread out fast in the first 12 hrs after gel embedment. NHLFs pull and align thick collagen bundles, result in straightened collagen bundles. FIG. 16C depicts the projected entire well tilescan imaging at 12 hrs and 36 hrs post gel embedment, only channel of MDA-MB-231s is shown. The same ROI was taken across two timepoints which allows accurate track of MDA-MB-231s′ colony expansion in 24 hrs. In MDA-MB-231+NHLF coculture condition, MDA-MB-231 and NHLF were seeded at 200K/ml and 50K/ml, respectively. FIG. 16D depicts quantification of MDA-MB-231 colony expansion among all six conditions, “MG_NorCol_12 hrs” indicates MDA-MB-231(lifeact-GFP) in normal collagen post 12 hrs gel embedment. “MG ThickCol_12hrs” indicates MDA-MB-231(lifeact-GFP) in thick collagen post 12 hrs gel embedment. “MG_F_ThickCol_12hrs” indicates MDA-MB-231(lifeact-GFP) cocultured with NHLF in thick collagen post 12 hrs gel embedment. The other three conditions are the same except for data taken at 36 hrs post gel embedment. FIG. 16E depicts long and thick collagen bundles can be formed when a high density of NHLFs are present (600 K/ml). In addition, fast MDA-MB-231 (lifeact-GFP) migration can be observed along this highly stretched “collagen bridge”.

FIG. 17 comprises FIGS. 17A to 17C and depicts images from videos. FIG. 17A depicts MDA-MB-231s′ (life-act,GFP) migration in normal collagen versus thick collagen on day0 and day1. FIG. 17B depicts MDA-MB-231s′(life-act,GFP) migration in thick collagen, zoomed-in view. Left, MDA-MB-231s demonstrate different behaviors within thick collagen patch versus on the boundary. Right: MDA-MB-231s usually demonstrate invasive phenotype in thick collagen. FIG. 17C depicts MCF10As migration in thick collagen, 1 mg/ml and 2 mg/ml normal collagen on day0 and day1. MCF10As demonstrate higher migration activity in thick collagen.

FIG. 18 comprises FIGS. 18A and 18B and depicts images from videos. FIG. 18A depicts MDA-MB-231s′(life-act,GFP) migration with drug treatment in normal versus thick collagen on day 0 and day 1. FIG. 18B depicts MDA-MB-231s′(life-act,GFP) migrate fast when cocultured with NHLF in thick collagen. Dynamic collagen pulling from NHLFs can be seen.

FIG. 19 comprises FIGS. 19A to 19G and depicts tunable collagen island architectures mimicking tissue topology. FIG. 19A depicts collagen architecture in tissue is diverse. Images are representative of normal (top) and diseased (bottom) breast, liver, and lung tissue. Scale bar is 20 um. FIG. 19B depicts schematic detailing collagen island assembly protocol. FIG. 19C depicts collagen island architecture schematic as a function of shear frequency and architecture imaged with confocal microscopy (stained collagen in cyan) and scanning electron microscopy to visualize macro, meso, and nanoscale. Scale bar in the second row is 100 μm. Scale bars in the third row and corresponding inset are 50 μm and 30 μm, respectively. Scale bar in the fourth row is 20 μm. FIG. 19D depicts pore area distribution for each island architecture. Right of dotted line indicates frequency of larger pore areas and therefore sparser collagen. FIG. 19E depicts cross sectional distribution of collagen architecture in a randomly selected location on confocal images. FIG. 19F depicts island area measured from confocal imaging, mean±sem. FIG. 19G depicts Young's modulus of island gels measured from shear rheometry. N≥3, mean±sd., *p<0.05; **, p<0.01; ***p<0.0001 by one-way ANOVA.

FIG. 20 comprises FIGS. 20A to 20C and depicts mechanical properties of collagen islands are tunable. FIG. 20A depicts strain sweeps of island gels. Differential shear modulus K as function of shear strain. Corresponding rate of strain stiffening (dK/dE) and maximum shear modulus for each architecture. N>3, mean±sem. FIG. 20B depicts average normalized stress relaxation tests at different strains. Blue is 60%, red is 30%, and purple is 10% strain. Black line indicates decreasing half max relaxation time as hold strain increases. FIG. 20C depicts Δ_{1/2} values for each island architecture at each given strain. While Δ_{1/2} decreases with increasing strain, there is no significant change in Δ_{1/2} values as a result of island architecture introduction. N≥3, mean±sd. *p<0.05 by one-way ANOVA.

FIG. 21 comprises FIGS. 21A to 21E and depicts cell contractile behavior is modulated by heterogeneous architecture. FIG. 21A depicts a schematic of gel compaction for MSCs (6×10⁵ cells/ml) for varied island architectures over 7 days. N≥3, mean±sem. FIG. 21B depicts a representative timelapse of MSCs in smaller gel volumes over 12 hours. Scalebar is 500 μm. FIG. 21C depicts representative zoomed in images of MSCs in smaller gel volumes over 12 hours. White arrows indicate plastic gel tracks formed between cells. Yellow arrows indicate island architecture movement. Scalebar is 500 μm. FIG. 21D depicts representative images and kymograph for MSCs cultured in (i) normal or (ii) mid shear island gels. Yellow arrow indicates dynamic protrusion and white lines indicate island architecture movement. Scale bar is 30 μm. FIG. 21E depicts gel compaction for MSCs (6×10 5 cells/ml) for varied island architectures over 7 days. N≥3, mean±sem. FIG. 21F depicts 7 day time course of MSCs cultured in each island architecture. FIG. 21G depicts quantified gel compaction area at days 1,2,3 and 7 for each island gel. N≥3, mean±sd. Magenta is stained collagen and cyan is MSC-LifeAct cells. *p<0.05; **, p<0.01; ***p<0.0001 by one-way ANOVA.

FIG. 22 comprises FIGS. 22A to 22F and depicts MSC migratory and differentiation behavior is modulated by island architecture. FIG. 22A depicts representative 3D track reconstructions for cell migration in island architectures. FIG. 22B depicts mean squared displacements, mean speeds, sphericity, and migration track length for migrating cells. N=3, n >200 cells. FIG. 22C depicts representative images of alkaline phosphatase staining (blue), indicating early osteogenic differentiation, for MSC cultured in island gels for 7 days. Scale bar is 200 μm. FIG. 22D depicts corresponding ALP (+) fraction of cells. n≥3 images from 3 independent replicates. FIG. 22E depicts representative images of RUNX2 (green), DAPI (cyan), and F-actin (magenta) of MSCs cultured in island gels for 7 days. FIG. 22F depicts corresponding RUNX2 nuclear to cytoplasmic ratio of cells cultured in each island gel. N=3, n>53 cells. Scale bar is 30 microns. mean±sd*p<0.05; **, p<0.01; ***p<0.0001 by one-way ANOVA.

FIG. 23 comprises FIGS. 23A to 23E and depicts iPSCs undergo mesodermal transitions in island architecture. FIG. 23A depicts representative images of iPSCs grown in normal or 5 second islands in co-gels with either 20% Matrigel by volume or 50% Matrigel by volume over 7 days. Scale bar is 200 μm. FIG. 23B depicts circularity and solidity of iPSC aggregates over 7 days and significantly decreases in matrigel:island cogels. FIG. 23C depicts quantified solidity, circularity, and % aggregates with low circularity (<0.7) after 7 days of culture. N=3, n>5 aggregates, mean±sd. FIG. 23D depicts representative immunofluorescent images of 50% Matrigel:Normal and 50% Matrigel:Island gels stained for DAPI (cyan), F-actin (magenta), Nanog (yellow) and ZO-1 (green) after 7 days of culture. iPSCs cultured in island co gels lose their steminess and apical-basal polarity indicated by loss of Nanog and ZO-1, respectively. FIG. 23E depicts after 7 days of culture, iPSCs show mesodermal differentiation in 50% Matrigel:Island gels indicated by nuclear localization of (left) Snail and (right) Brachyury (insets). Scale bar is 100 μm. Inset scale bar is 30 μm. mean±sd*p<0.05; **, p<0.01; ***p<0.0001 by one-way ANOVA. FIG. 23F depicts corresponding nuclear to cytoplasmic ratio of SNAIL (left) and Brachyury (right). Scale bar is 100 μm. Inset scale bar is 30 μm. mean±sd*p<0.05; **, p<0.01; ***p<0.001; ***p<0.0001 by one-way ANOVA.

FIG. 24 comprises FIGS. 24A and 24B. FIG. 24A depicts COMSOL simulations of both pipette tip and tube during shearing process. As shearing frequency decreases, shear stress introduced into the system also decreases. FIG. 24B depicts instantaneous energy introduced into the system as a function of time. Bars above indicate full period length for each condition. Maximum shear stress, total energy, and total power delivered can be calculated for each shear period.

FIG. 25 depicts velocity profiles of COMSOL simulations for high shear, mid shear, and high shear islands respectively.

FIG. 26 depicts macroscale imaging of island architectures showing the characteristic size of each island gel. 3D projection of island architectures indicating that island topography is distributed throughout the gel. Scale bars are 250 μm (top) and 100 μm (bottom).

FIG. 27 depicts Fourier analysis of line intensity. Line intensity profiles are taken on tile scan images that are ˜400 μm in length. This demonstrates island gels showing bias towards lower frequency signals, indicating inhomogeneity.

FIG. 28 comprises FIGS. 28A and 28B. FIG. 28A depicts confocal microscopy of high shear, mid shear, and low shear island gels (top) and the same gels that were centrifuged and then resuspended in 50% less solution. Scale bar is 50 μm for large image and 30 μm for the inset. FIG. 28B depicts mid shear island gels resuspended in 10 mg/ml fibrin in increasing packing fraction. Fibrin is imaged with reflectance microscopy and collagen (gray) is stained. Scale bar is 100 μm.

FIG. 29 comprises FIGS. 29A to 29C and depicts automated collagen island mixer schematic and output. FIG. 29A depicts schematic illustrating motorized movement to create island architectures. Collagen solution is placed in a steel holder at the bottom. Stepper motor moves syringe plunger up and down at specified frequency to create collagen islands. FIG. 29B depicts a photo of automated collagen mixer. FIG. 29C depicts collagen mixer recapitulates increasing island size as shear frequency decreases. Scale bar is 100 μm.

FIG. 30 depicts the differential shear modulus, K=∂τ/∂γ, at maximum strain amplitude is used to quantify the stiffness of the matrix.

FIG. 31 depicts failure strain of collagen island gels from strain sweeps. N>3, mean±sd *p<0.05 by one-way ANOVA.

FIG. 32 depicts full stress relaxation profiles. Average stress relaxation profiles indicating relaxation modulus (top) and raw stress (bottom) of at least 3 replicates per strain and per condition.

FIG. 33 depicts normalized stress relaxation. Average stress relaxation profiles indicating normalized relaxation modulus of at least 3 replicates per strain and per condition.

FIG. 34 depicts stress relaxation max stress plotted as a function of strain values. N=3, mean±sd*p<0.05 by one-way ANOVA.

FIG. 35 depicts Day 2 Immunofluorescence of MSCs subjected to Gel Compaction. F-Actin (green), DNA (magenta), and collagen (reflectance, gray) shown for normal, 5 second, and 10 second island gels. Scale bar is 100 μm.

FIG. 36 comprises FIGS. 36A and 36B. FIG. 36A depicts gel compaction of 4 mg/ml collagen which compacts to a higher plateau area. FIG. 36B depicts gel compaction area on Day 1, 2, 3 and 7 for all island gels plus inhibitors and 4 mg/ml gels. N=3. mean±sd*p<0.05; **, p<0.01; ***p<0.0001 by one-way ANOVA.

FIG. 37 depicts polydopamine coating protocol for collagen gel adhesion.

FIG. 38 comprises FIGS. 38A and 38B. FIG. 38A depicts day 1 mean squared displacements, mean speeds, sphericity, and migration track length for MSCs in various architectures. FIG. 38B depicts day 1, mean speeds, sphericity, and migration track length for migrating cells treated with various cytoskeletal inhibitors. N=3, n>90 cells. mean±sd*p<0.05; ** p<0.01; ***p<0.0001 by one-way ANOVA.

FIG. 39 comprises FIGS. 39A to 39H. FIG. 39A depicts day 6 mean migration track length for migrating cells treated with various cytoskeletal inhibitors. N=3 gels, n>90 cells. FIG. 39B depicts cell density of MSCs in different architectures determined from immunofluorescence imaging. FIG. 39C depicts representative images of Alizarin Red and Oil Red staining for MSCs cultured in island gels for 7 days. Scale bar is 100 μm. FIG. 39D depicts representative images of ALP staining staining for MSCs cultured in island gels for 7 days treated with various cytoskeletal inhibitors. Scale bar is 100 μm. FIG. 39E depicts ALP positive fraction of MSCs treated with cytoskeletal inhibitors and agonists. Spread area of MSCs in different architectures. FIG. 39F depicts RUNX2 nuclear to cytoplasmic ratio of MSCs treated with cytoskeletal inhibitors and agonists. FIG. 39G depicts spread area of MSCs in different architectures. FIG. 39H depicts cell spread area of MSCs treated with cytoskeletal inhibitors and agonists. N =3 gels, ells mean±sd*p<0.05; **, p<0.01; ***p<0.0001 by one-way ANOVA.

FIG. 40 depicts ALP Positive Cells vs Collagen Concentration. Amount of ALP positive cells increases with increasing concentration of collagen. mean±sd*p<0.05; **, p<0.01; ***p<0.0001 by one-way ANOVA.

FIG. 41 depicts representative images of MSCs treated with RhoA or RhoGTPase agonist. Cells treated with RhoGTPase agonist form connected network structure. Scale bar is 200 μm.

FIG. 42 comprises FIGS. 42A and 42B. FIG. 42A depicts iPSCs cultured in pure 3D Matrigel system stained for DAPI (cyan), F-actin (magenta), vimentin (green), and Oct4 (yellow) for 7 days. iPSC aggregates show maintained stemness in 100% Matrigel system. FIG. 42B depicts iPSCs grown in 50% Matrigel:Island gels show EMT transition indicated by expression of vimentin. Only invasive cells on the periphery of lumen in 50% Matrigel: Normal show vimentin expression. Scale bar is 100 μm.

FIG. 43 depicts quantified solidity and circularity after 2,3,5, and 7 days of culture. N=3, n>5 aggregates, mean±sd. *p<0.05; **, p<0.01; ***p<0.0001 by one-way ANOVA.

FIG. 44 depicts qPCR of stem cells cultured in various architectures. Relative mRNA expression of iPSCs cultured in matrigel with or without island architectures. iPSCs lose pluripotency markers and gain mesodermal markers (TBX6) when cultured in island gels. N>3. mean±sd*p<0.05; **, p<0.01; ***p<0.0001 by one-way ANOVA.

### DETAILED DESCRIPTION

It is to be understood that the figures and descriptions of the present invention have been simplified to illustrate elements that are relevant for a clear understanding of the present invention, while eliminating, for the purpose of clarity, many other elements found in related systems and methods. Those of ordinary skill in the art may recognize that other elements and/or steps are desirable and/or required in implementing the present invention. However, because such elements and steps are well known in the art, and because they do not facilitate a better understanding of the present invention, a discussion of such elements and steps is not provided herein. The disclosure herein is directed to all such variations and modifications to such elements and methods known to those skilled in the art.

### Definitions

Unless defined otherwise, all technical and scientific terms used herein have the same meaning as commonly understood by one of ordinary skill in the art to which this invention belongs. Although any methods and materials similar or equivalent to those described herein can be used in the practice or testing of the present invention, exemplary methods and materials are described.

As used herein, each of the following terms has the meaning associated with it in this section.

The articles “a” and “an” are used herein to refer to one or to more than one (i.e., to at least one) of the grammatical object of the article. By way of example, “an element” means one element or more than one element.

“About” as used herein when referring to a measurable value such as an amount, a temporal duration, and the like, is meant to encompass variations of ±20%, ±10%, ±5%, ±1%, and ±0.1% from the specified value, as such variations are appropriate.

Throughout this disclosure, various aspects of the invention can be presented in a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 2.7, 3, 4, 5, 5.3, 6 and any whole and partial increments therebetween. This applies regardless of the breadth of the range.

The term “extracellular matrix” as used herein refers to a scaffold in a cell's external environment with which the cell interacts via specific cell surface receptors. The extracellular matrix serves many functions, including, but not limited to, providing support and anchorage for cells, segregating one tissue from another tissue, and regulating intracellular communication. The extracellular matrix is composed of an interlocking mesh of fibrous proteins and glycosaminoglycans (GAGs). Examples of fibrous proteins found in the extracellular matrix include collagen, elastin, fribronectin, and laminin. Examples of GAGs found in the extracellular matrix include proteoglycans (e.g., heparin sulfate), chondroitin sulfate, keratin sulfate, and non-proteoglycan polysaccharide (e.g., hyaluronic acid).

As used herein, the term “spheroid” refers to microtissues of cells growing and/or interacting within their surroundings in all three dimensions in an artificially-created environment. Such microtissues can comprise a plurality of homotypic or heterotypic cells, preferably mammalian cells, more preferably human cells. Such 3-D cell cultures more closely resemble the in vivo surroundings of the cells as compared to 2-D cell cultures. Spheroids provide a more accurate model system for cellular, physiological and/or pharmaceutical studies than cells grown in conventional two-dimensional cultures.

As used herein, the term “fibrotic cell” is defined as an injured cell that has a lower capability to conduct or generate an electrical impulse as compared to normal, healthy cells. Such fibrotic cell has lost its capability to conduct or generate an electrical impulse. Fibrotic cells are obtained in various ways. Surrounding cells can be rendered fibrotic by heating them with a heating element with a temperature of at least 55° C., or cooling them with a cooling element with a temperature of at most −75° C.

“Collagen” is a structural protein found in connective tissue; it frequently takes the form of fibrils arranged in a triple helix. Fibrillar types of collagen include Types I, II, III, V, and XI. Type I collagen makes up a great deal of the organic part of bone as well as being found in skin, tendons, blood vessels, and organs, while type III collagen is commonly found near or with type I. On the other hand, cartilage is composed primarily of type II collagen. Other types of cartilage are less common and may be found in membranes, on cell surfaces, and associated with hair and placental structures.

### Methods of Making Collagen Bundles

In one aspect, the present invention relates to a method of generating thickened collagen bundles, the method comprising the steps of preparing a neutralized collagen gel sample; warming the gel sample; and agitating the warmed gel sample until the gel sample has a turbidity above background. In one embodiment, the method generates clusters. Any embodiment involving a bundle is applicable to any embodiment involving a cluster, and vice-versa.

In one embodiment, the method may be employed to prepare collagen through the modulation of collagen-based ECM scaffolds by disrupting the gelation process. In one embodiment, the method is performed manually. In one embodiment, the method is performed using an automated device. In one embodiment, the method is performed using a mixing device.

In one embodiment, the collagen is derived from rat tail. In one embodiment, the collagen is derived from bovine. In one embodiment, the collagen has been neutralized.

In one embodiment, the method involves cooling the collagen gel to about 0° C. In one embodiment, the method involves cooling the collagen gel to about 0° C. to 5° C. In one embodiment, the method involves cooling the collagen gel to about 5° C. to 10° C. In one embodiment, the method involves cooling the collagen gel to about 10° C. to 15° C. In one embodiment, the method involves cooling the collagen gel to about 15° C. to 20° C. In one embodiment, the method involves cooling the collagen gel to about 20° C. to 25° C.

In one embodiment, the method involves warming the cooled collagen gel to about 0° C. In one embodiment, the method involves warming the cooled collagen gel to about 5° C. In one embodiment, the method involves warming the cooled collagen gel to about 10° C. In one embodiment, the method involves warming the cooled collagen gel to about 15° C. In one embodiment, the method involves warming the cooled collagen gel to about 20° C. In one embodiment, the method involves warming the cooled collagen gel to about 25° C. In one embodiment, the method involves warming the cooled collagen gel to ambient temperature.

In one embodiment, the method involves letting the warmed collagen gel sit for 6 to 7 minutes. In one embodiment, the method involves letting the warmed collagen gel sit for 6 to 8 minutes. In one embodiment, the method involves letting the warmed collagen gel sit for 6 to 9 minutes. In one embodiment, the method involves letting the warmed collagen gel sit for 6 to 10 minutes. In one embodiment, the method involves letting the warmed collagen gel sit for 5 to 10 minutes. In one embodiment, the method involves letting the warmed collagen gel sit for 5 to 8 minutes. In one embodiment, the method involves letting the warmed collagen gel sit for 5 to 9 minutes.

In one embodiment, the method involves warming the gel after sitting to about 25° C. In one embodiment, the method involves warming the gel after sitting to about 30° C. In one embodiment, the method involves warming the gel after sitting to about 31° C. In one embodiment, the method involves warming the gel after sitting to about 32° C. In one embodiment, the method involves warming the gel after sitting to about 33° C. In one embodiment, the method involves warming the gel after sitting to about 34° C. In one embodiment, the method involves warming the gel after sitting to about 35° C. In one embodiment, the method involves warming the gel after sitting using heat from a hand.

In one embodiment, the method involves pipetting the warmed gel. In one embodiment, the method involves mixing the warmed gel with a pipette. In one embodiment, the method involves mixing the warmed gel with an automated device. In several embodiments, mixing the warmed gel increases turbidity. In one embodiment, the warmed gel is mixed until it has a turbidity above background.

In one embodiment, the step of agitating the warmed neutralized collagen gel is performed for over about 5 minutes. In one embodiment, the step of agitating the warmed neutralized collagen gel is performed for over about 10 minutes. In one embodiment, the step of agitating the warmed neutralized collagen gel is performed for over about 15 minutes. In one embodiment, the step of agitating the warmed neutralized collagen gel is performed for over about 20 minutes. In one embodiment, the step of agitating the warmed neutralized collagen gel is performed for over about 30 minutes. In one embodiment, the step of agitating the warmed neutralized collagen gel is performed for over about 40 minutes. In one embodiment, the step of agitating the warmed neutralized collagen gel is performed for over about 50 minutes. In one embodiment, the step of agitating the warmed neutralized collagen gel is performed for over about 60 minutes.

In one embodiment, the method provides disconnected collage clusters. In one embodiment, the method provides loosely connected collagen clusters. In one embodiment, the method further comprises the step of mixing collagen clusters with cells. In one embodiment, the method provides a cell and collagen cluster mixture.

In one embodiment, the method further comprises the step of placing the cell and collagen cluster mixture onto a low adhesion plate. In one embodiment, the method further comprises the step of placing the cell and collagen cluster mixture into a well. In one embodiment, the method further comprises the step of forming the cell and collagen cluster mixture into hanging droplets. In one embodiment, the step of placing the cell and collagen cluster mixture is performed with a pipette. In one embodiment, the step of forming the cell and collagen cluster mixture is performed with a pipette.

In one embodiment, the cell and collagen cluster mixture self-aggregates. In one embodiment, the cell and collagen cluster mixture undergo cell aggregation. In one embodiment, the cell and collagen cluster mixture comprises a structure selected from the group consisting of compact cell-collagen clusters, spheroids, and fibrotic cell clusters.

In one embodiment, the neutralized collagen sample has a collagen concentration of about 1 mg/mL to about 10 mg/mL. In one embodiment, the neutralized collagen sample has a collagen concentration of about 1 mg/mL to about 9 mg/mL. In one embodiment, the neutralized collagen sample has a collagen concentration of about 1 mg/mL to about 8 mg/mL. In one embodiment, the neutralized collagen sample has a collagen concentration of about 1 mg/mL to about 7 mg/mL. In one embodiment, the neutralized collagen sample has a collagen concentration of about 1 mg/mL to about 6 mg/mL. In one embodiment, the neutralized collagen sample has a collagen concentration of about 1 mg/mL to about 5 mg/mL. In one embodiment, the neutralized collagen sample has a collagen concentration of about 1 mg/mL to about 4 mg/mL. In one embodiment, the neutralized collagen sample has a collagen concentration of about 1 mg/mL to about 3 mg/mL. In one embodiment, the neutralized collagen sample has a collagen concentration of about 1 mg/mL to about 2 mg/mL. In one embodiment, the neutralized collagen sample has a collagen concentration of about 2 mg/mL to about 10 mg/mL. In one embodiment, the neutralized collagen sample has a collagen concentration of about 2 mg/mL to about 9 mg/mL. In one embodiment, the neutralized collagen sample has a collagen concentration of about 2 mg/mL to about 8 mg/mL. In one embodiment, the neutralized collagen sample has a collagen concentration of about 2 mg/mL to about 7 mg/mL. In one embodiment, the neutralized collagen sample has a collagen concentration of about 2 mg/mL to about 6 mg/mL. In one embodiment, the neutralized collagen sample has a collagen concentration of about 2 mg/mL to about 5 mg/mL. In one embodiment, the neutralized collagen sample has a collagen concentration of about 2 mg/mL to about 4 mg/mL. In one embodiment, the neutralized collagen sample has a collagen concentration of about 2 mg/mL to about 3 mg/mL.

In one embodiment, the neutralized collagen gel sample comprises a collagen selected from the group consisting of type I collagen, type II collagen, type III collagen, type IV collagen, type XI collagen, thick collagen, hydrolyzed collagen, mammal collagen, powdered collagen, and synthetic collagen.

In one embodiment, turbidity of the gel is measured with a microplate reader. In one embodiment, the microplate reader is pre-heated. In one embodiment, the colorimetric readout is monitored. In one embodiment, the turbidity is determined by the colorimetric readout.

In one embodiment, the microplate reader is pre-heated to 30° C. In one embodiment, the microplate reader is pre-heated to 31° C. In one embodiment, the microplate reader is pre-heated to 32° C. In one embodiment, the microplate reader is pre-heated to 33° C. In one embodiment, the microplate reader is pre-heated to 34° C. In one embodiment, the microplate reader is pre-heated to 35° C. In one embodiment, the microplate reader is pre-heated to 36° C. In one embodiment, the microplate reader is pre-heated to 37° C. In one embodiment, the microplate reader is pre-heated to 38° C. In one embodiment, the microplate reader is pre-heated to 39° C. In one embodiment, the microplate reader is pre-heated to 40° C.

In one embodiment, the colorimetric readout is recorded every 1 minute for 20 minutes. In one embodiment, the colorimetric readout is recorded every 1 minute for 25 minutes. In one embodiment, the colorimetric readout is recorded every 1 minute for 30 minutes. In one embodiment, the colorimetric readout is recorded every 1 minute for 35 minutes. In one embodiment, the colorimetric readout is recorded every 1 minute for 40 minutes. In one embodiment, the colorimetric readout is recorded every 2 minutes for 20 minutes. In one embodiment, the colorimetric readout is recorded every 5 minutes for 20 minutes.

In one embodiment, the stirring of the warmed gel results in a turbidity around 0.4-0.5. In one embodiment, the stirring of the warmed gel results in a turbidity around 0.4-0.6. In one embodiment, the stirring of the warmed gel results in a turbidity around 0.4-0.7. In one embodiment, the stirring of the warmed gel results in a turbidity around 0.4-0.8. In one embodiment, the stirring of the warmed gel results in a turbidity around 0.4-0.9. In one embodiment, the stirring of the warmed gel results in a turbidity around 0.4-1.0.

In another aspect, the present invention provides thick collagen bundle gels. In one embodiment, the thick collagen bundle gels are globally softer than normal collagen gels. In one embodiment, the thick collagen bundle gels display viscoelastic behavior similar to that of normal collagen gels. In one embodiment, the thick collagen bundle gels display higher frequency dependent behavior than normal collagen gels during bulk rheometry.

In one embodiment, the thick collagen bundle gels possess stiff mesoscopic clusters. In one embodiment, the thick collagen bundle gels have tunable mesoscopic patterns. In one embodiment, the thick collagen bundle gels contain pores. In one embodiment, the thick collagen bundle gels display macroporosity. In one embodiment, the thick collagen bundle gels display mesoporosity.

In one embodiment, the diameters of the pores range from 1 to 10 μm. In one embodiment, the diameters of the pores range from 1 to 20 μm. In one embodiment, the diameters of the pores range from 1 to 30 μm. In one embodiment, the diameters of the pores range from 1 to 40 μm. In one embodiment, the diameters of the pores range from 1 to 50 μm. In one embodiment, the diameters of the pores range from 10 to 20 μm. In one embodiment, the diameters of the pores range from 10 to 30 μm. In one embodiment, the diameters of the pores range from 10 to 40 μm. In one embodiment, the diameters of the pores range from 10 to 50 μm. In one embodiment, the diameters of the pores range from 10 to 60 μm. In one embodiment, the diameters of the pores range from 10 to 70 μm. In one embodiment, the diameters of the pores range from 10 to 80 μm. In one embodiment, the diameters of the pores range from 10 to 90 μm. In one embodiment, the diameters of the pores range from 10 to 100 μm. In one embodiment, the diameters of the pores range from 100 to 200 μm. In one embodiment, the diameters of the pores range from 200 to 300 μm. In one embodiment, the diameters of the pores range from 300 to 400 μm. In one embodiment, the diameters of the pores range from 400 to 500 μm.

In one embodiment, the thick collagen bundle gels can be incorporated with several cell types. Exemplary cell types include, but are not limited to, stem cells and tissue cells to study development. In one embodiment, cancer cells are embedded into the hydrogels to study dissemination in physiologically relevant settings. In one embodiment, cancer cells are embedded into the hydrogels to study migration dynamics in physiologically relevant settings. In one embodiment, the hydrogels are applied to study differentiation in vitro. In one embodiment, the thick collagen bundle gels can be incorporated with co-gel biomaterials.

In one embodiment, the thick collagen bundle gels are used in three-dimensional tissues. In one embodiment, the thick collagen bundle gels are used in a vascular organoid.

In one embodiment, the thick collagen bundle gels are used to induce stem cell differentiation. In one embodiment, the thick collagen bundle gels are used to induce mesodermal differentiation.

In one embodiment, the thick collagen bundle gels can serve as bioactive cues to influence the behavior of cells. In one embodiment, the thick collagen bundle gels facilitate cell infiltration. In one embodiment, the thick collagen bundle gels are employed in in situ injectables. In one embodiment, the thick collagen bundle gels are employed in vaccines. In one embodiment, the thick collagen bundle gels are used as microporous gels.

In one embodiment, the thick collagen bundle gels are used for immune stimulation. In one embodiment, the thick collagen bundle gels are used for wound healing. In one embodiment, the thick collagen bundle gels are used for vascularization.

In one embodiment, the hydrogels contain mesoscaled architectures. In one embodiment, the hydrogels contain macroscaled architectures. In one embodiment, the hydrogels are clusters.

### Collagen Island Hydrogels

In another aspect, the present invention provides collagen island hydrogels. In one embodiment, the collagen island hydrogels require only mechanical force to form. In one embodiment, the collagen island hydrogels are formed using shear. In one embodiment, the collagen island hydrogels have tunable island size. In one embodiment, the island size is dependent on mechanical shear frequency. In one embodiment, the collagen island hydrogels can be isolated from solution and resuspended in other natural ECM-derived matrices. In one embodiment, the collagen island hydrogels have tunable topography. In one embodiment, the collagen island hydrogels have tunable mechanical properties.

In one embodiment, the collagen island hydrogels are formed via mixing. In one embodiment, the collagen island hydrogels are formed via mixing for about 1 second to about 10 minutes. In one embodiment, the collagen island hydrogels are formed via mixing for about 1 second to about 10 seconds. In one embodiment, the collagen island hydrogels are formed via mixing for about 1 second to about 20 seconds. In one embodiment, the collagen island hydrogels are formed via mixing for about 1 second to about 30 seconds. In one embodiment, the collagen island hydrogels are formed via mixing for about 1 second to about 40 seconds. In one embodiment, the collagen island hydrogels are formed via mixing for about 1 second to about 50 seconds. In one embodiment, the collagen island hydrogels are formed via mixing for about 1 second to about 60 seconds. In one embodiment, the collagen island hydrogels are formed via mixing for about 1 second to about 2 minutes. In one embodiment, the collagen island hydrogels are formed via mixing for about 1 second to about 3 minutes. In one embodiment, the collagen island hydrogels are formed via mixing for about 1 second to about 4 minutes. In one embodiment, the collagen island hydrogels are formed via mixing for about 1 second to about 5 minutes. In one embodiment, the collagen island hydrogels are formed via mixing for about 1 second to about 6 minutes. In one embodiment, the collagen island hydrogels are formed via mixing for about 1 second to about 7 minutes. In one embodiment, the collagen island hydrogels are formed via mixing for about 1 second to about 8 minutes. In one embodiment, the collagen island hydrogels are formed via mixing for about 1 second to about 9 minutes. In one embodiment, the collagen island hydrogels are formed via mixing for about 1 minute to about 10 minutes.

In one embodiment, the area of the collagen islands ranges from 1 to 1500 μm². In one embodiment, the area of the collagen islands ranges from 1 to 1000 μm². In one embodiment, the area of the collagen islands ranges from 1 to 750 m². In one embodiment, the area of the collagen islands ranges from 1 to 500 m². In one embodiment, the area of the collagen islands ranges from 1 to 1000 μm 2 . In one embodiment, the area of the collagen islands ranges from 1 to 450 μm². In one embodiment, the area of the collagen islands ranges from 1 to 400 μm². In one embodiment, the area of the collagen islands ranges from 1 to 350 m². In one embodiment, the area of the collagen islands ranges from 1 to 300 m². In one embodiment, the area of the collagen islands ranges from 1 to 250 m². In one embodiment, the area of the collagen islands ranges from 1 to 200 μm². In one embodiment, the area of the collagen islands ranges from 1 to 150 m². In one embodiment, the area of the collagen islands ranges from 1 to 100 μm². In one embodiment, the area of the collagen islands ranges from 1 to 50 μm².

In several embodiments, the collagen island hydrogels mimic the heterogeneous mesoscopic features seen in tissue. In one embodiment, the collagen island hydrogels are compatible with modular assembly for tissue engineering. In one embodiment, the collagen island hydrogels act as bioactive cues. In one embodiment, the collagen island hydrogels modulate stem cell behavior. In one embodiment, the collagen island hydrogels can promote MSC osteogenesis. In one embodiment, the collagen island hydrogels can direct spontaneous iPSC differentiation. In one embodiment, the collagen island hydrogels can induce cellular self-assembly processes. In one embodiment, the collagen island hydrogels induce microtissue fabrication. In one embodiment, the collagen island hydrogels induce tissue morphogenesis. In one embodiment, the collagen island hydrogels direct cell differentiation. In one embodiment, the collagen island hydrogels direct physical cues in regenerative applications.

In one embodiment, the collagen island hydrogels are used in a biomimetic material. In one embodiment, the biomimetic material displays strain stiffening. In one embodiment, the biomimetic material displays stress relaxation.

In one embodiment, the collagen island hydrogels can be incorporated with several cell types. Exemplary cell types include, but are not limited to, stem cells and tissue cells to study development. In one embodiment, cancer cells are embedded into the hydrogels to study dissemination in physiologically relevant settings. In one embodiment, cancer cells are embedded into the hydrogels to study migration dynamics in physiologically relevant settings. In one embodiment, the hydrogels are applied to study differentiation in vitro. In one embodiment, the collagen island hydrogels can be incorporated with co-gel biomaterials.

In one embodiment, the collagen island hydrogels are used in three-dimensional tissues. In one embodiment, the collagen island hydrogels are used in a vascular organoid.

In one embodiment, the collagen island hydrogels are used to induce stem cell differentiation. In one embodiment, the collagen island hydrogels are used to induce mesodermal differentiation.

In one embodiment, the collagen island hydrogels can serve as bioactive cues to influence the behavior of cells. In one embodiment, the collagen island hydrogels facilitate cell infiltration. In one embodiment, the collagen island hydrogels are employed in in situ injectables. In one embodiment, the collagen island hydrogels are employed in vaccines. In one embodiment, the collagen island hydrogels are used as microporous gels.

In one embodiment, the collagen island hydrogels are used for immune stimulation. In one embodiment, the collagen island hydrogels are used for wound healing. In one embodiment, the collagen island hydrogels are used for vascularization.

In one embodiment, the hydrogels contain mesoscaled architectures. In one embodiment, the hydrogels contain macroscaled architectures. In one embodiment, the hydrogels are clusters. In one embodiment, the hydrogels are thick tortuous fiber bundles.

### EXPERIMENTAL EXAMPLES

The invention is further described in detail by reference to the following experimental examples. These examples are provided for purposes of illustration only and are not intended to be limiting unless otherwise specified. Thus, the invention should in no way be construed as being limited to the following examples, but rather, should be construed to encompass any and all variations which become evident as a result of the teaching provided herein.

Without further description, it is believed that one of ordinary skill in the art can, using the preceding description and the following illustrative examples, make and utilize the compositions of the present invention and practice the claimed methods. The following working examples, therefore, specifically point out various embodiments of the present invention, and are not to be construed as limiting in any way the remainder of the disclosure.

### Collagen Bundle Structure

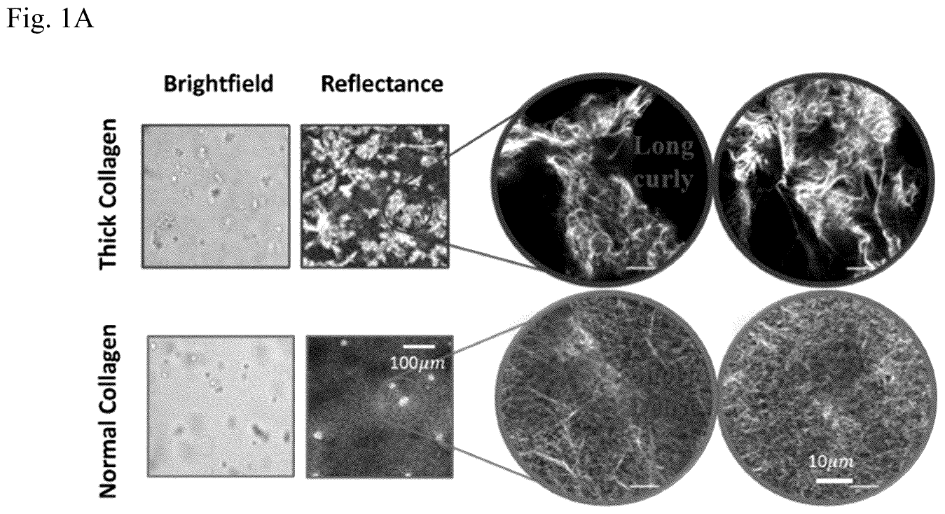

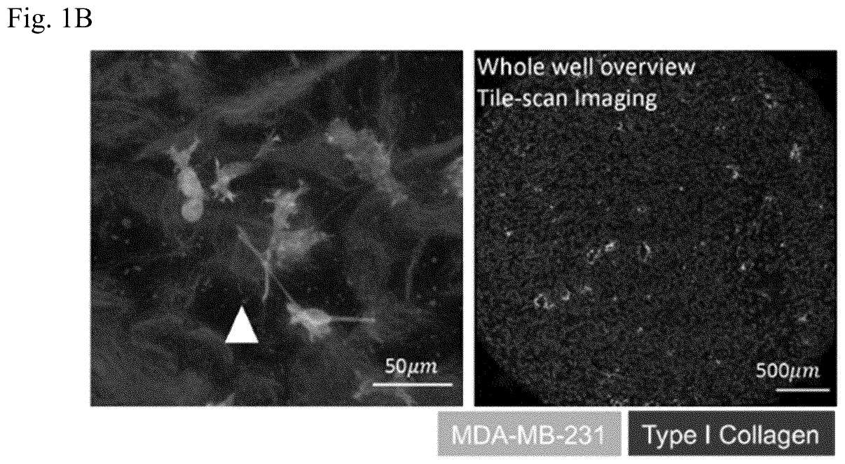

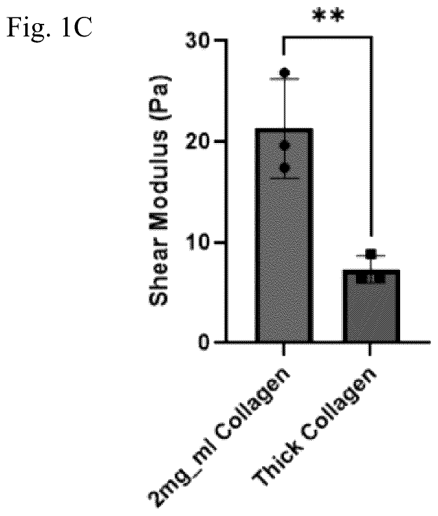

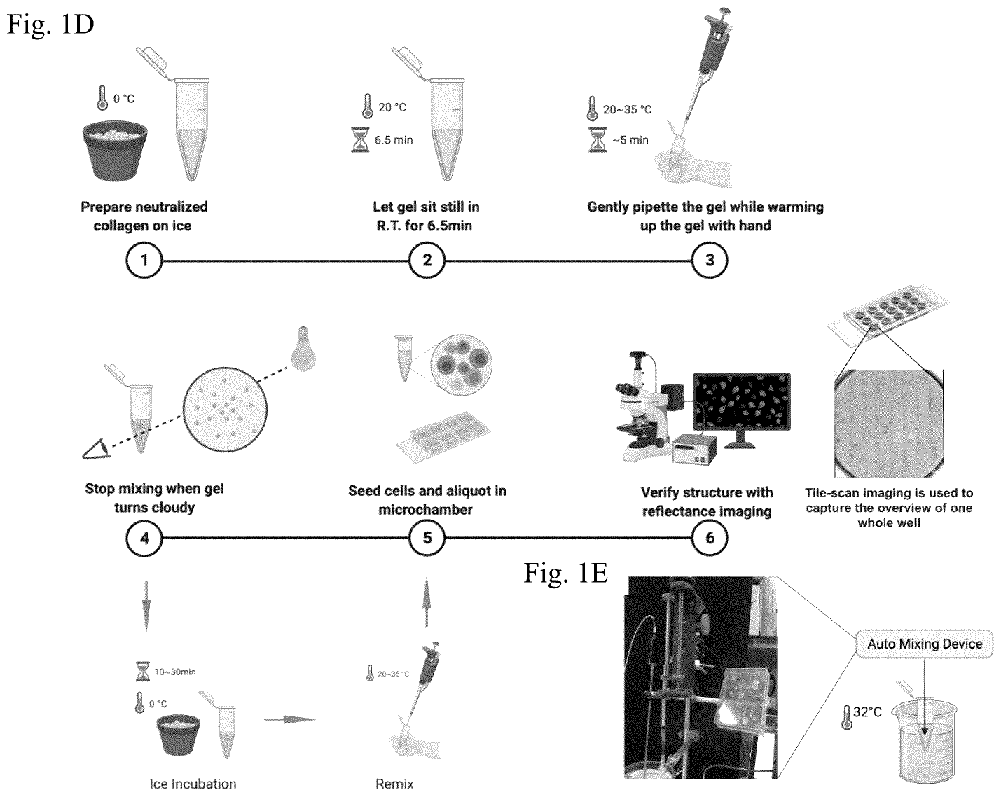

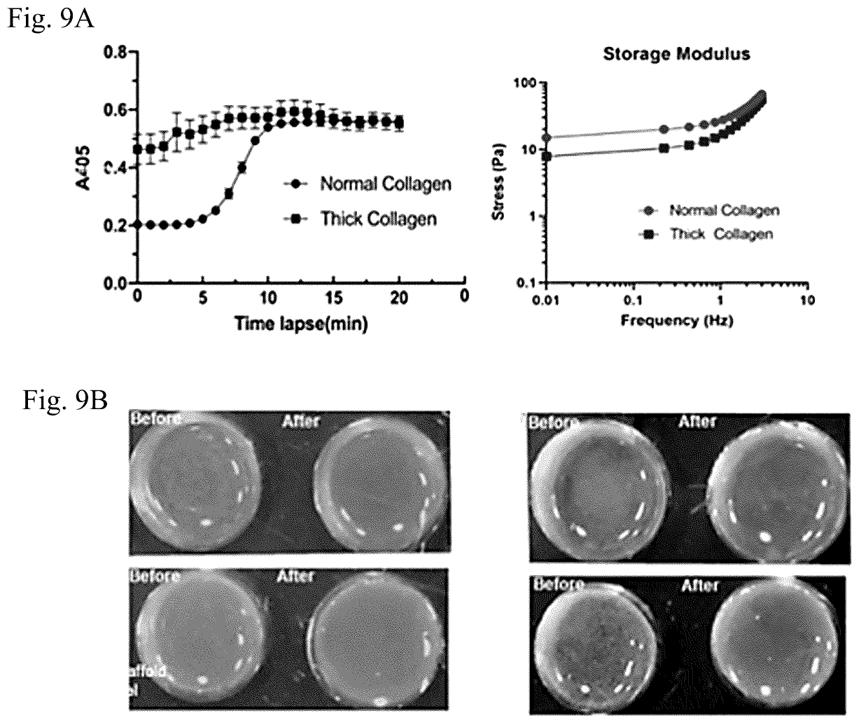

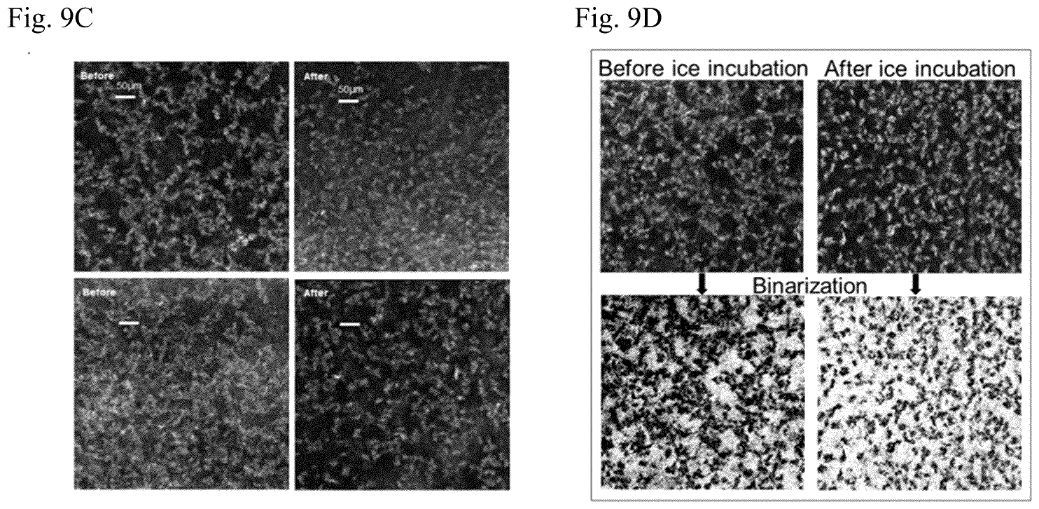

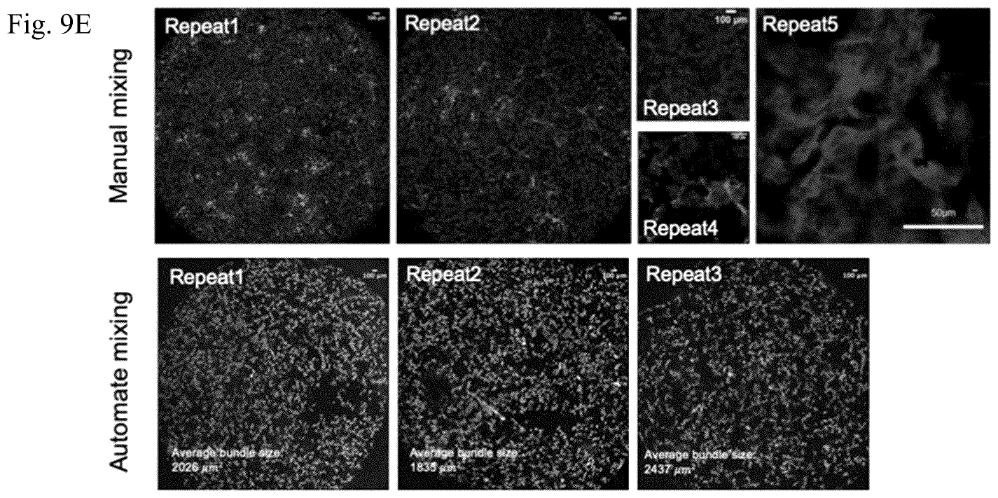

Thickened collagen bundles were long and curly in appearance, when compared with short, dense collagen networks gels prepared without mechanical disturbance, i.e. normal collagen, as shown in FIG. 1A. Collagen bundles imposed limited physical restraints, as both 3D collagen networks supported cell growth and provided a good mechanical scaffold. As for its detailed structure, thickened collagen patches were interconnected by thin collagen fibers, which were not clearly seen in reflectance imaging, but attached polystyrene beads indicated the existence of this loose network (FIG. 1B, left). Thick collagen patches varied in size, orientation, and micro-structure, providing local spatial heterogeneity for individual cells, but these patches were relatively uniformly distributed and separated by the loose collagen network (FIG. 1B, right). In addition, bulk rheometry revealed that thick collagen bundle gels are globally softer than normal collagen gels (FIG. 1C). Frequency sweeps revealed viscoelastic behavior in thick collagen gels, similar to that of normal collagen gels, but with slightly higher frequency dependent behavior (FIG. 9A, right). The generation of thickened collagen bundles did not require complex equipment, the overall process took 20 minutes to be completed and was reproducible. In manual fabrication (FIG. 1D), neutralized ice-cold collagen was first kept at room temperature for 6.5 minutes, then warmed up by fingertips while being stirred gently by a wide-bore pipette tip until turbidity reached between 0.4-0.5 (FIG. 9B). Similar thick collagen bundles structures were obtained between independent experiments, although a certain degree of variation can be seen (FIG. 9C, top row). To standardize the procedure, an automated homogenization device was developed and adapted to the protocol accordingly (FIG. 1E). With this device, thick collagen bundles were generated by mixing while heating ice-cold collagen gel at 32° C. for 4 min-5 min.

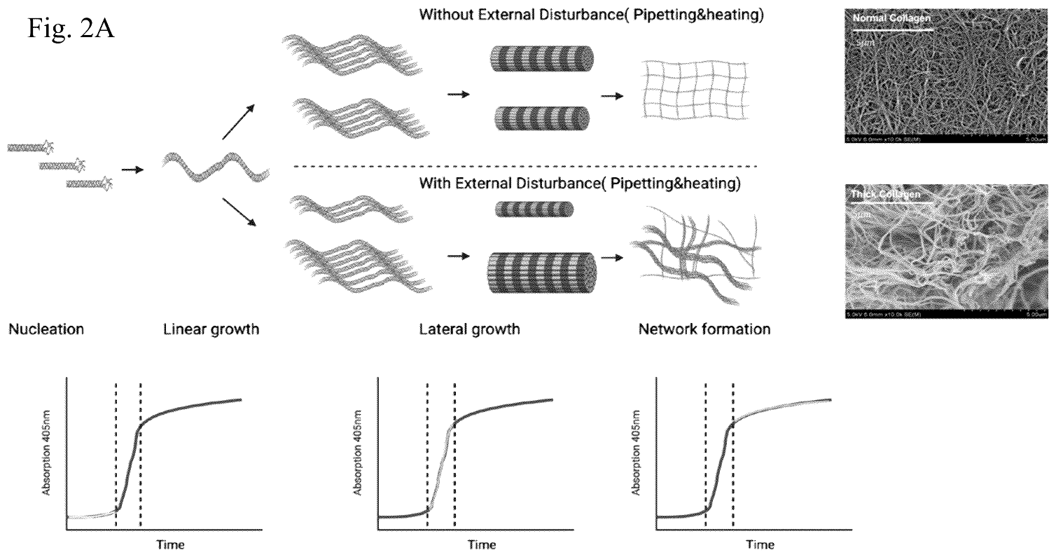

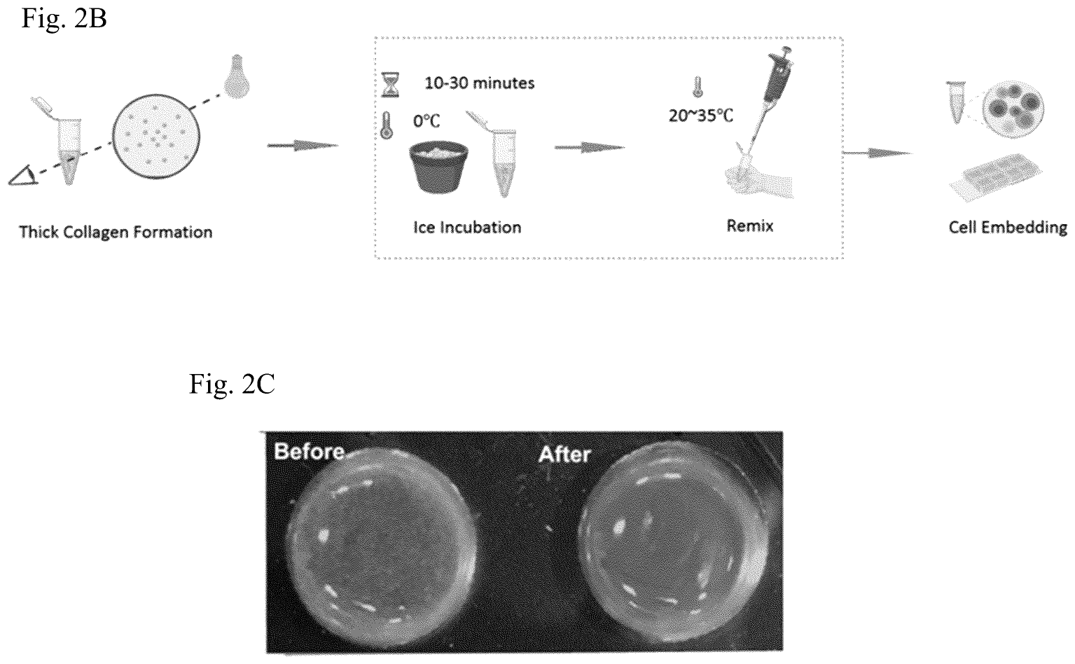

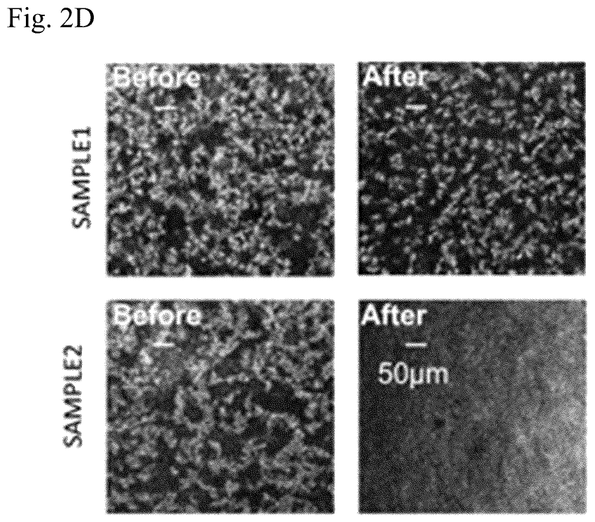

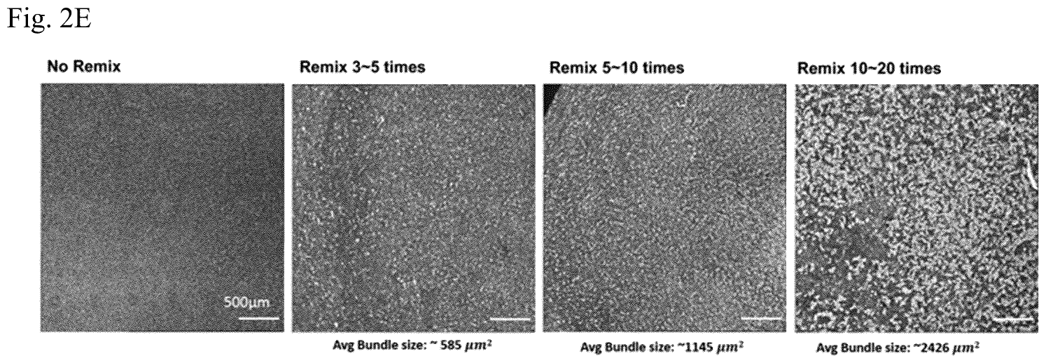

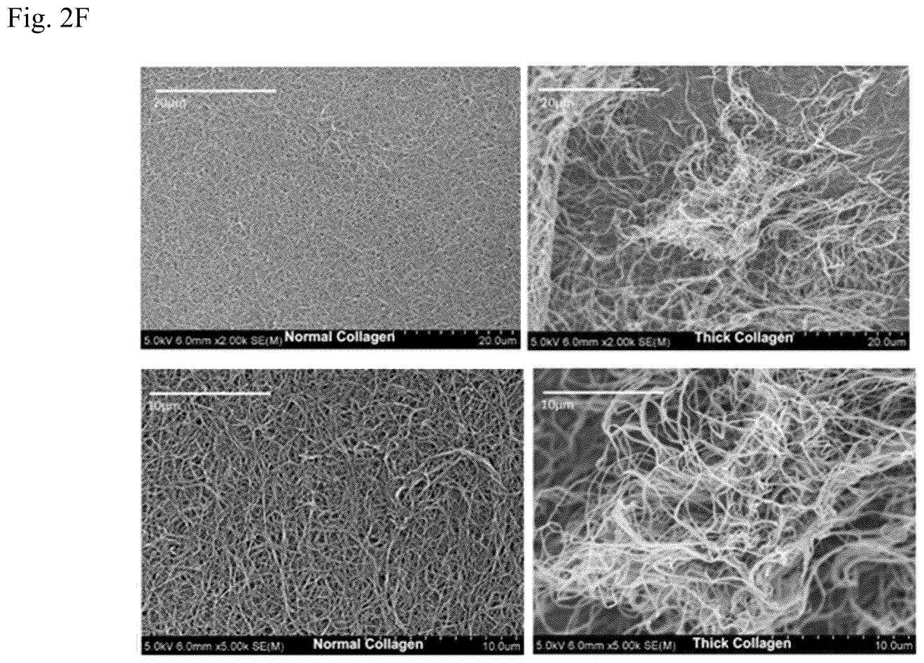

Previous studies have focused on the dynamics of fibrillar gel formation. The steps proposed for in vitro fibrillogenesis, portrayed in FIG. 2A, are nucleation, linear growth, and lateral growth (Sapudom et al., Biomaterials, 2019, 193, 47-57; Wood, Biochemical Journal 1960, 75, 598-605; Silver & Birk, Collagen and Related Research, 1983, 3, 393-405). The thickening of fibrils has often been reflected in the lag phase of the typical turbidity curve (Wood, Biochemical Journal, 1960, 75, 598-605; Silver & Birk, Collagen and Related Research 1983, 3, 393-405); Yang et al., Biomaterials, 2010, 31, 5678-5688). However, it has been suggested that the growth of visible particle-like structure is associated with the transition phase in the turbidity curve (Zhu & Kaufman, Biophysical Journal, 2014, 106, 1822-1831). Also, collagen precipitation can dissolve upon pH (Rosenblatt et al., Biomaterials, 1994, 15, 985-995) or temperature change (de Wild et al., Biophysical Journal, 2013, 105, 200-210; Wood, Biochemical Journal 1960, 75, 598-605; Silver & Birk, Collagen and Related Research, 1983, 3, 393-405), and this temperature-associated fibril instability may be governed by entropy. In this line, temperature tuning during fibrillogenesis has been used to control collagen pore size (Yang et al., Biomaterials 2010, 31, 5678-5688). Indeed, clusters in thick collagen structures were not thermally stable and may disassemble upon ice incubation (FIG. 2B, FIG. 2C, FIG. 9E). In addition to temperature, it is noted that frequency and strength of remixing between step 4 and step 5, as annotated in FIG. 2B, are critical for tuning the average size of collagen patches. Of note, the size of the final collagen patches was dependent on the degree of mixing (FIGS. 2D and 2E). However, over mixing often resulted in an ultra-fragile collagen network that collapsed upon PBS addition. The introduction of mechanical disturbance may have caused an uneven aggregation in gelation cores of collagen fibrils during lateral growth stage. For this, heating while mixing in the lateral growth stage may favor a more homogeneous distribution of these early gelation cores. Furthermore, 37° C. incubation propagated the early heterogeneity and resulted in thick collagen networks.

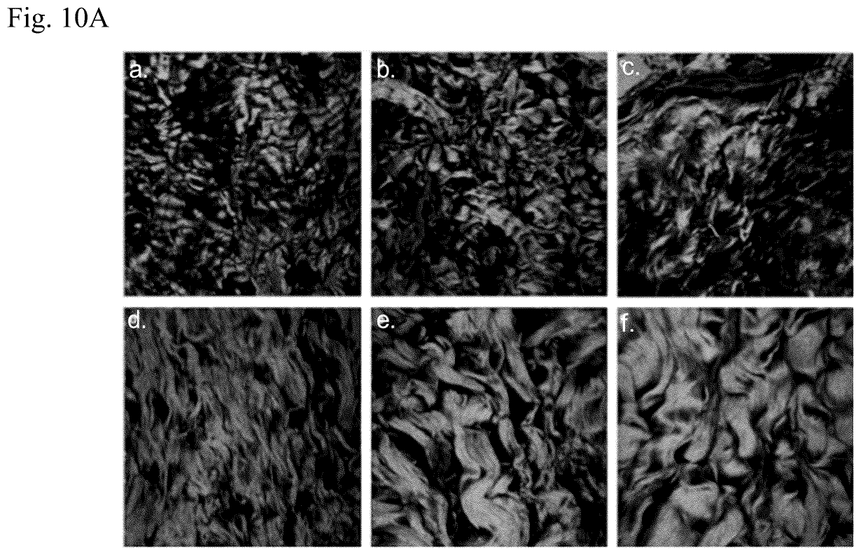

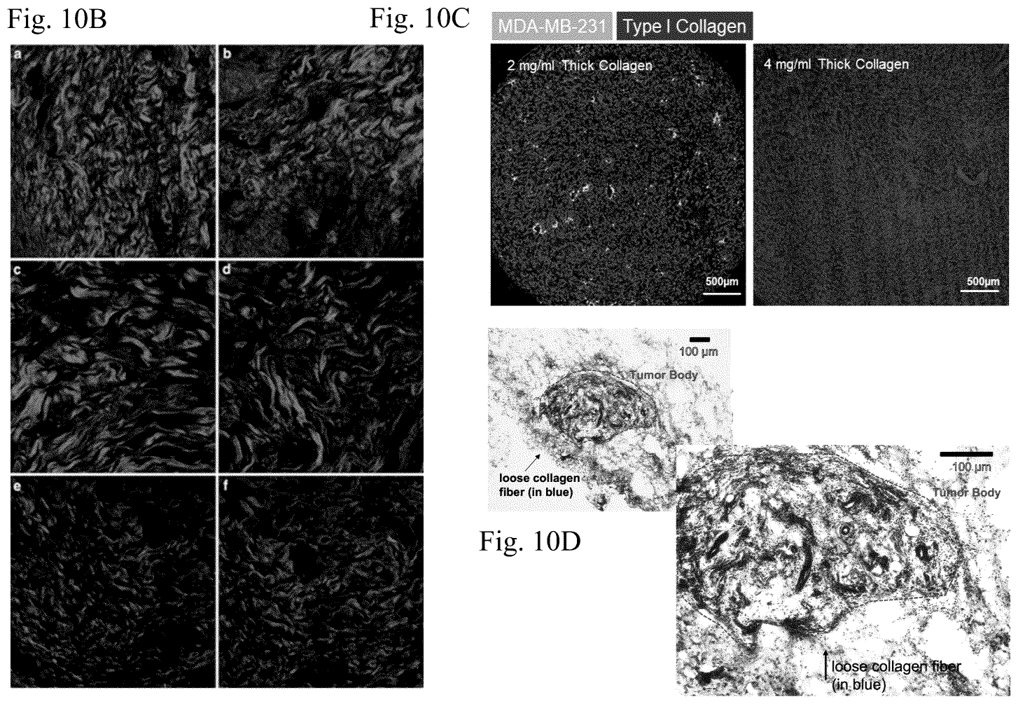

As shown in FIG. 2F, scanning electron microscopy (SEM) images of 2 mg/ml normal collagen and 2 mg/ml thick collagen were consistent with above-mentioned observations with confocal reflectance imaging (FIGS. 1A and 1B). Normal collagen is a network with dense and small pores, while thick collagen patches consist of long entangled and tortuous fibers. Interestingly, thick collagen seemed to highly mimic the architecture of collagen from human scars (FIG. 2B). Overall, different fibrillar collagen gel patterns can be created by modulating collagen concentration, temperature, and mixing (FIG. 10C).

MDA-MB-231 is a breast cancer triple-negative basal type cell line. It is highly aggressive and metastatic, featuring low-claudin (reduced cell-cell contact) and overexpression of ECM components and remodeling-related proteases, mesenchymal associated markers, and tumor favoring signals (Dai et al., Journal of Cancer 2017, 8, 3131-3141). In in vitro 3D models, MDA-MB-231 cells are highly sensitive to collagen alignment (Nuhn et al., Acta Biomaterialia, 2018, 66, 248-257; Han et al., Proceedings Of The National Academy Of Sciences Of The United States Of America, 2016, 113, 11208-11213), stiffness (Geiger et al., PloS One, 2019, 14, e0225215; Sapudom et al., Biomaterials, 2015, 52, 367-375) and steric hindrance, such as pore size (Wolf et al., The Journal Of Cell Biology, 2013, 201, 1069-1084). In vivo, MDA-MB-231 tumors preferentially metastasize to the bones, brain and lungs (Bos et al., Nature, 2009, 459, 1005-1009; Kang et al., Cancer Cell, 2003, 3, 537-549; Minn et al., The Journal of Clinical Investigation, 2005, 115, 44-55).

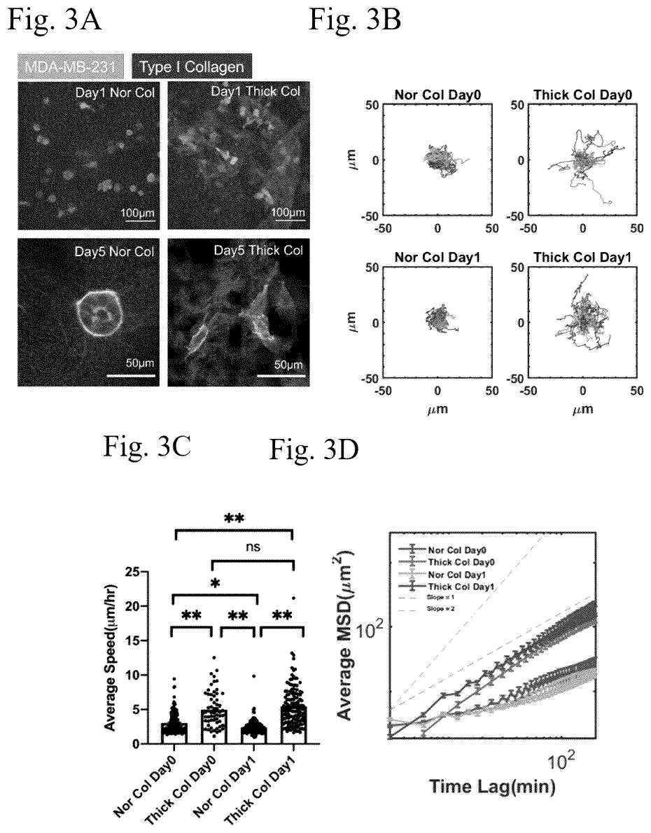

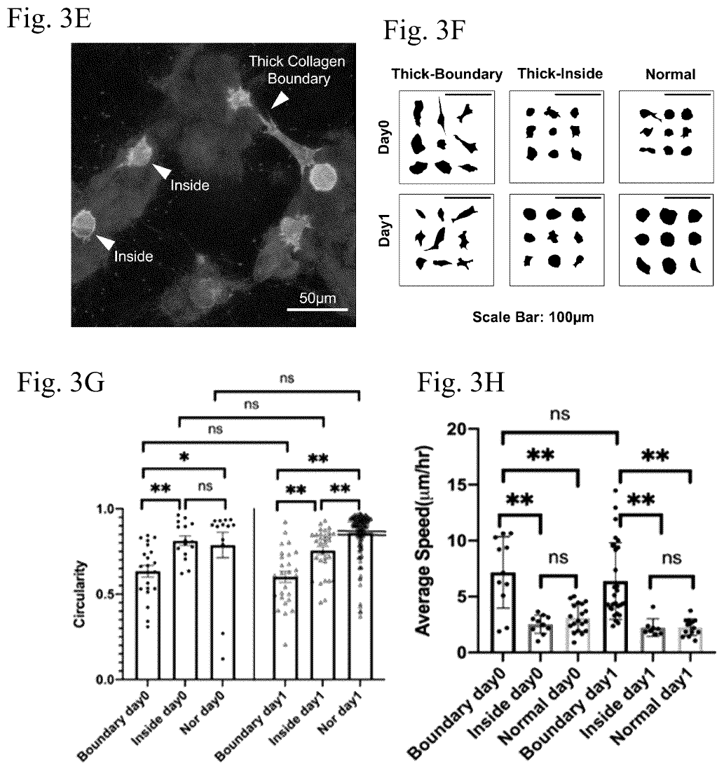

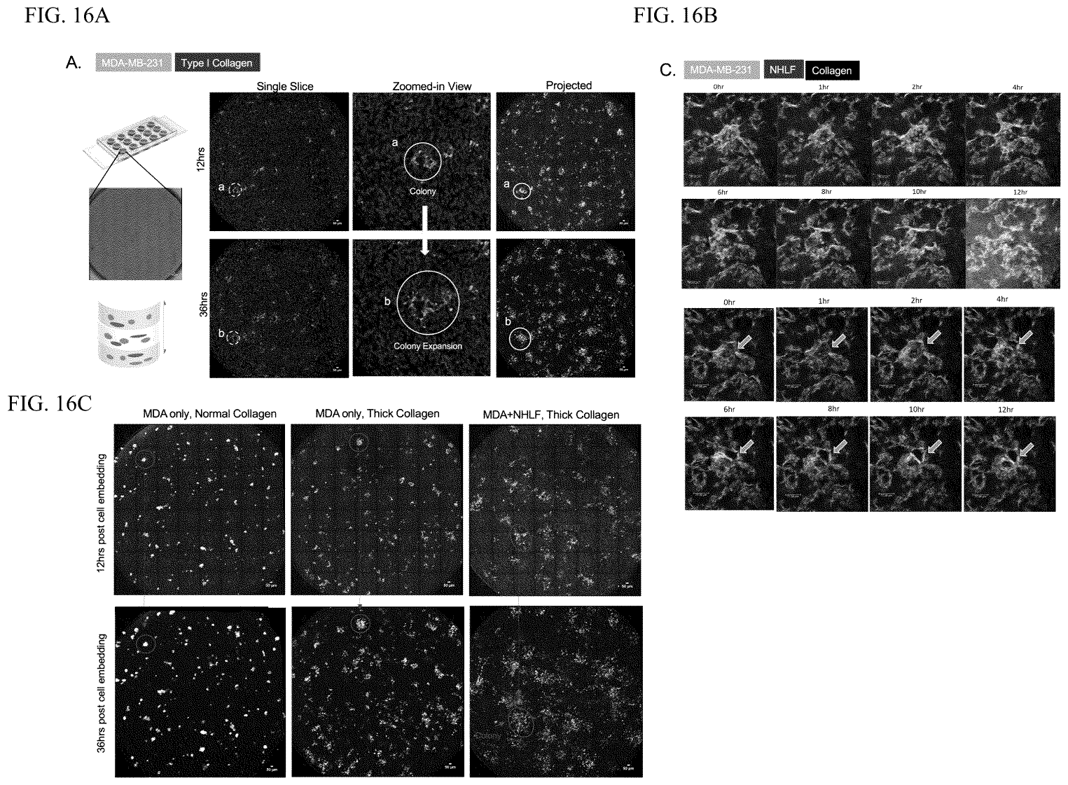

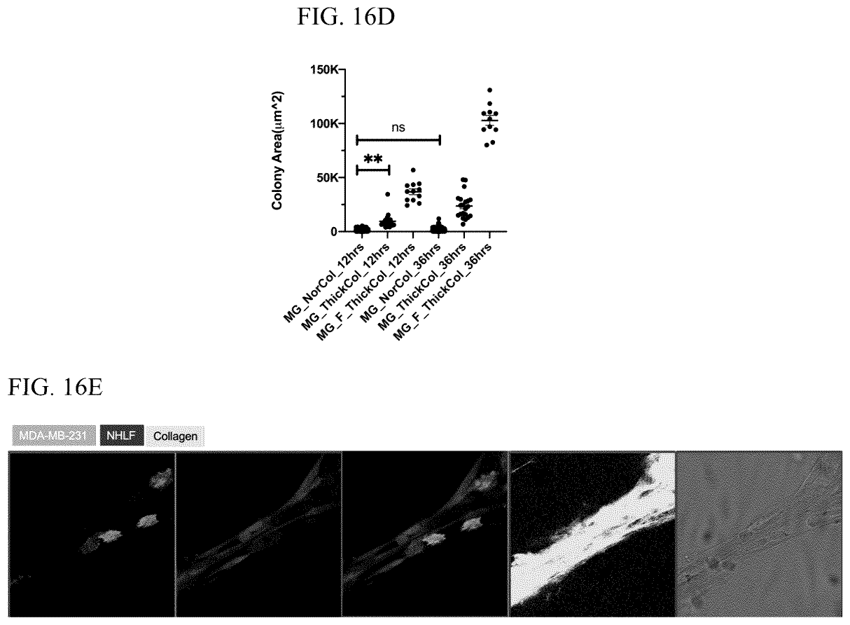

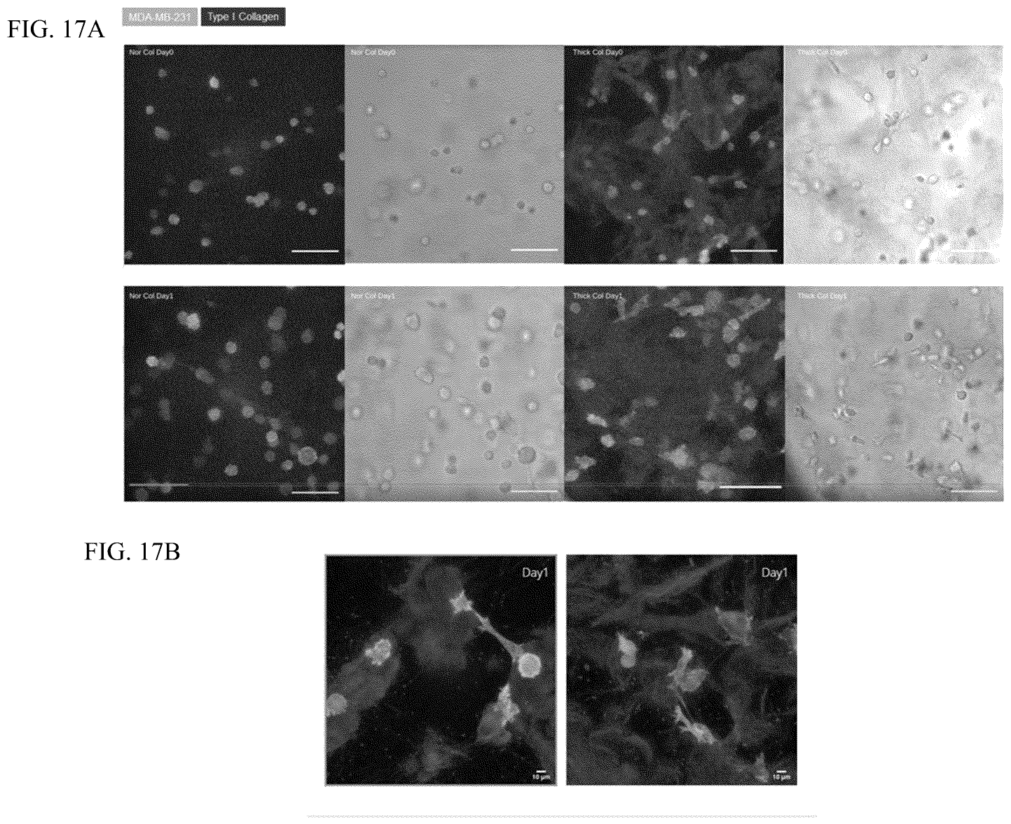

The thick collagen system was characterized by reduced physical hindrance, global softness, and local thickened fiber bundles. When embedded in the thick collagen gels, MDA-MB-231 cells responded rapidly during the first 24 h. In long term culture (up to day 5), MDA-MB-231 cells in normal collagen mostly grew into spherical cell clusters whereas the ones seeded in thick collagen remained isolated and rarely clustered (FIG. 3A, FIG. 11A). This observation can be further supported by overview of the tile scan imaging (FIG. 16A and FIG. 16B). Overall, when in thick collagen, MDA-MB-231 cells demonstrated higher migration (FIG. 3B), average speed (FIG. 3C, FIG. 11C) and persistence (as shown by the MSD and its logarithmic slope) (FIG. 3D) compared with cells in normal collagen on day 0 and day 1 (FIG. 17A). In addition, MDA-MB-231 cells often demonstrated two distinct morphological phenotypes depending on the position of cells in the thick collagen patches (FIG. 3E, FIG. 11B, FIG. 17B). MDA-MB-231 cells perching on the boundary of thick collagen bundles were often highly stretched, spindle-like, protruding towards nearby collagen patches (FIG. 3E, FIG. 11B-left). MDA-MB-231 cells located in the center of a thick collagen patch were more spherical, similar to cells in normal collagen (FIG. 3F, FIG. 3G). Consistent with morphological differences, MDA-MB-231 cells on the boundary displayed higher migration speed than cells in the middle of thick collagen patches (FIG. 3H).

Persistent cell migration in a 3D environment requires protrusive structures and actomyosin-mediated contractility (Pandya et al., Current Opinion in Cell Biology, 2017, 48, 87-96). The branching and elongation of protrusive structures often involves ARP2/3 and formin-nucleated actin polymerization (Pollard, Annual Review of Biophysics And Biomolecular Structure, 2007, 36, 451-477; Goode & Eck, Annual Review Of Biochemistry, 2007, 76, 593-627), whereas Rho-ROCK signaling is in the center of actomyosin-mediated cell contractility (Amano et al., Cytoskeleton, 2010, 67, 545-554). The Rho GTPases Rho, Rac and Cdc42 are major players in cytoskeletal regulation (Sadok & Marshall, Small GTPases, 2014, 5, e29710). In 3D ECMs, matrix metalloproteinases (MMPs) contribute to matrix degradation and remodeling, which allows cell migration (Singh et al., Frontiers in Molecular Biosciences, 2015, 2, 19; Wolf et al., The Journal Of Cell Biology, 2013, 201, 1069-1084).

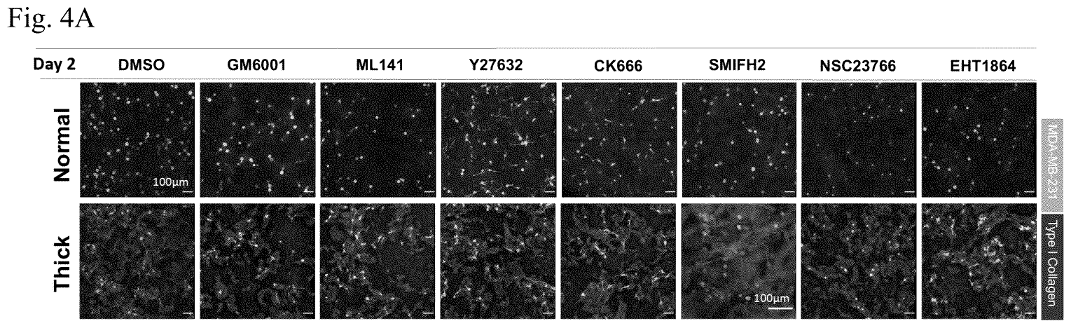

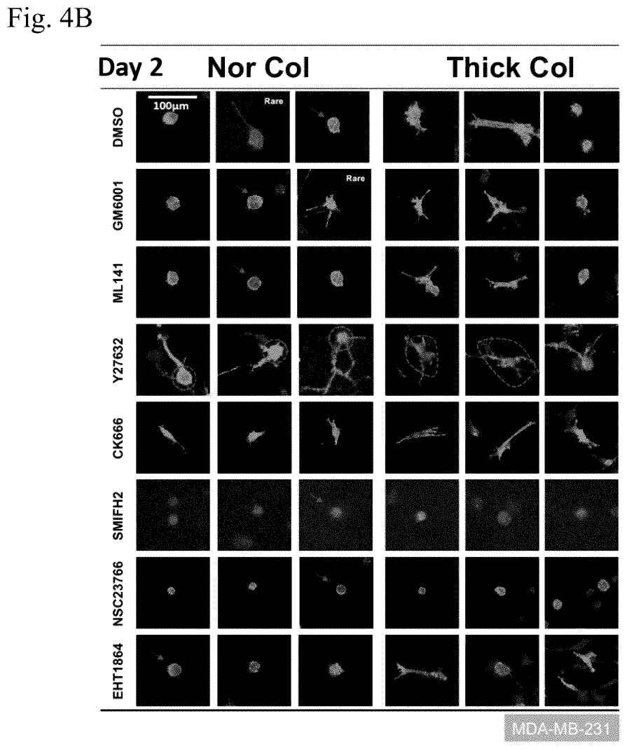

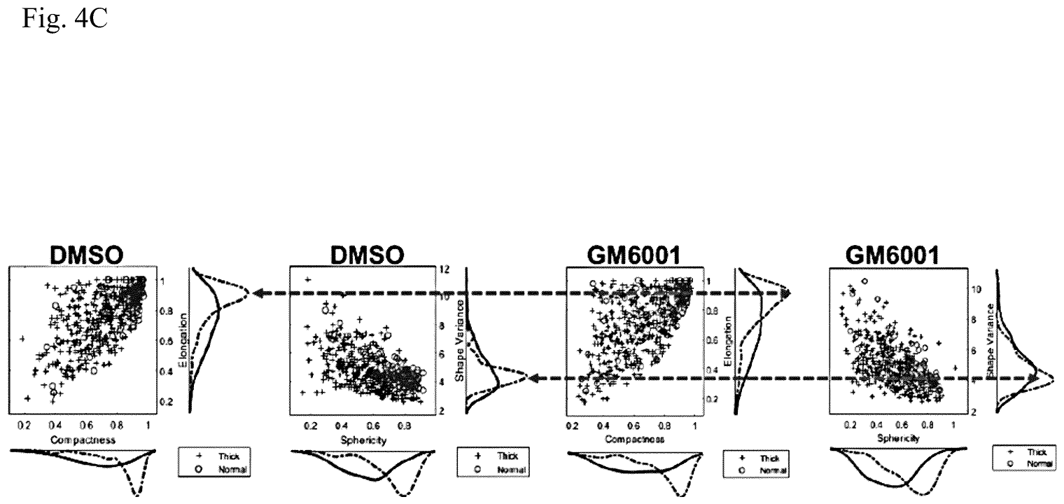

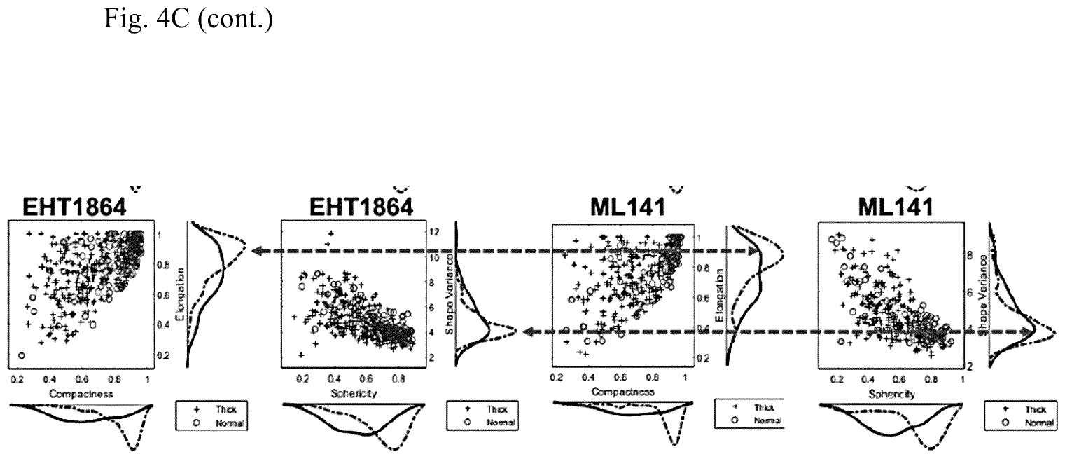

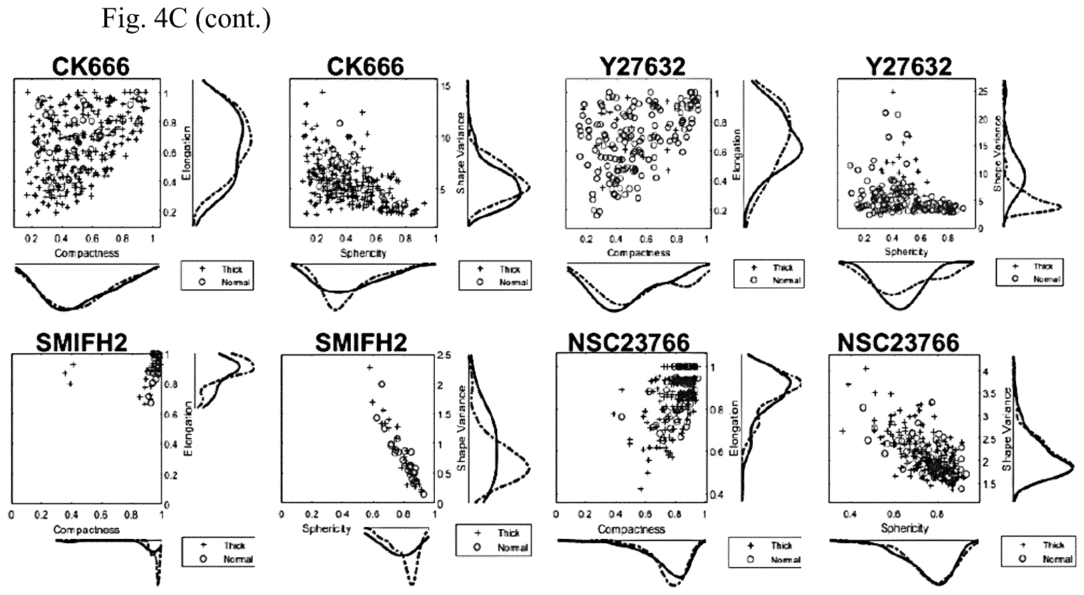

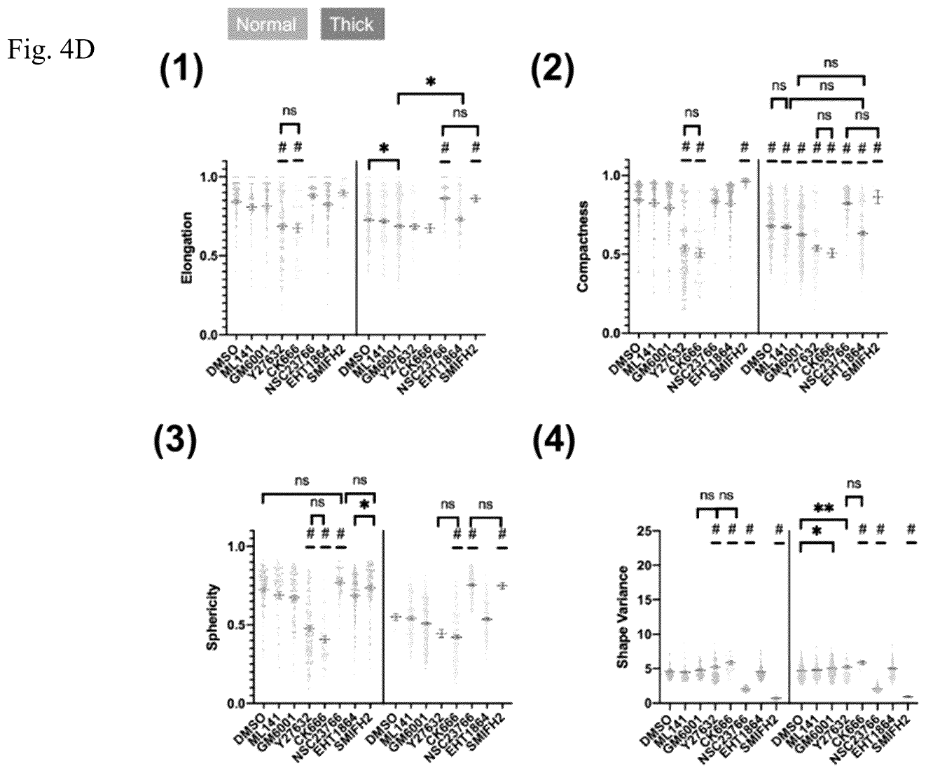

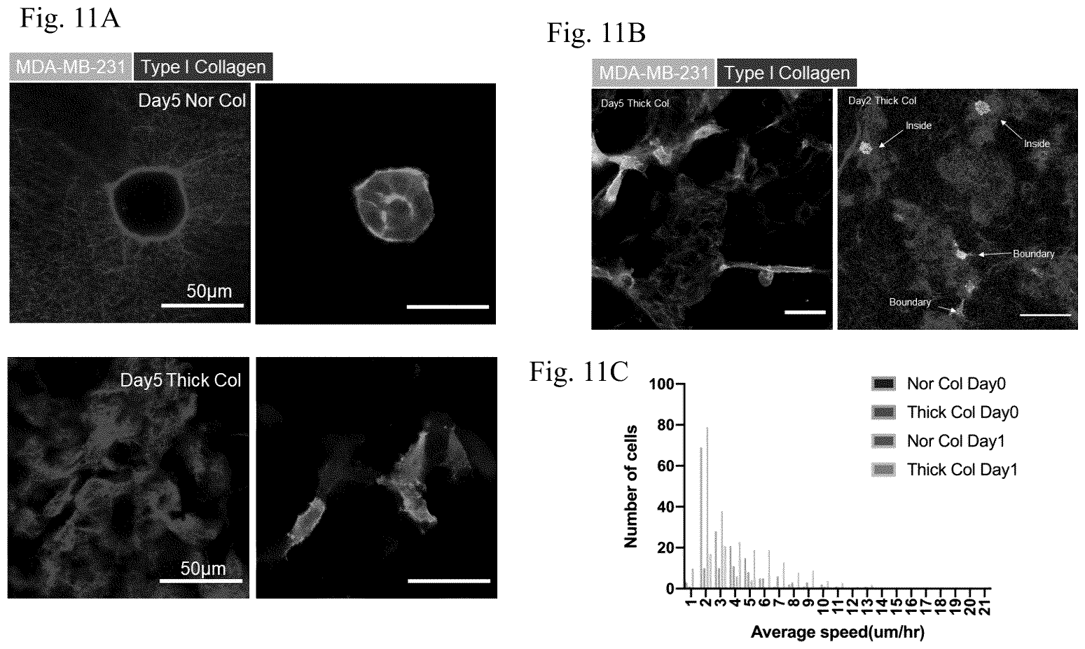

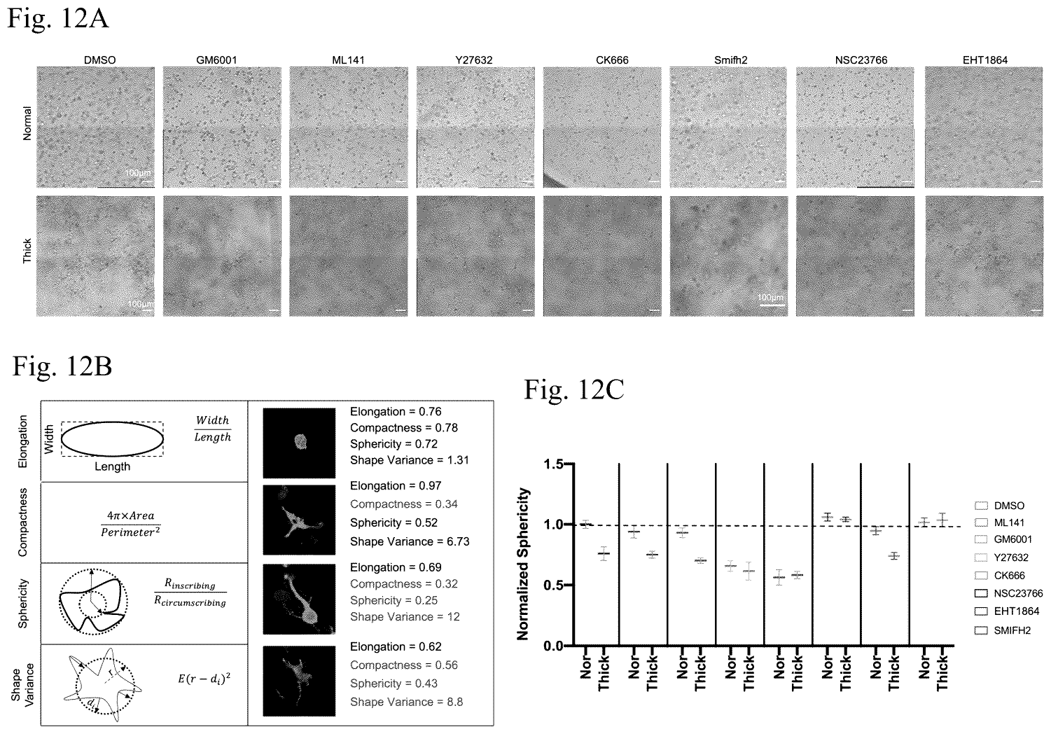

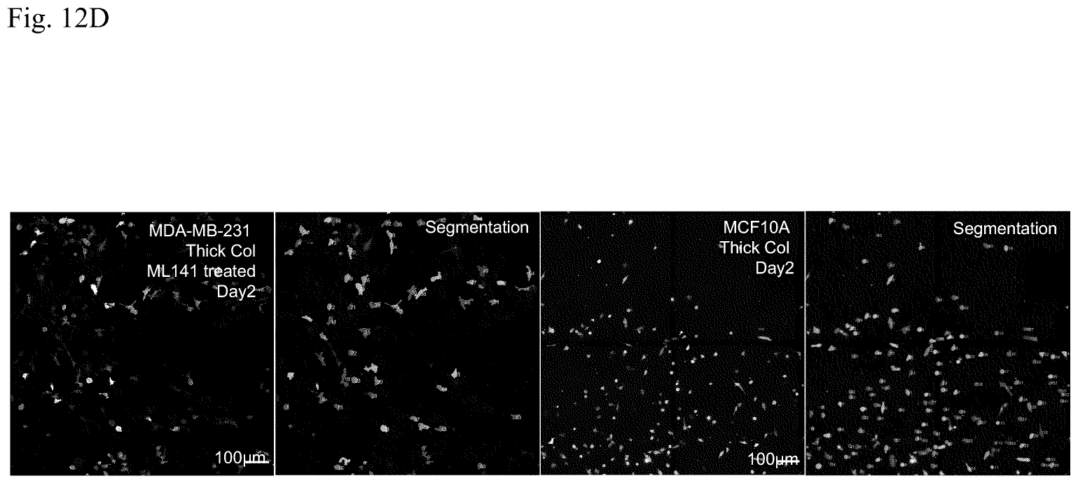

To investigate the specific molecular mechanism behind the elevated migration of MDA-MB-231 cells in thick collagen, the following drugs were tested in these gels: pan-MMP inhibitor GM6001 (20 μM), potent selective Cdc42 inhibitor ML141 (10 μM), ROCK inhibitor Y27632 (30 μM), ARP2/3 inhibitor CK666 (50 μM), formin inhibitor SMIFH2 (50 μM) and Rac1 inhibitors NSC23766 (100 μM) and EHT1864 (20 μM). First cell morphology was traced and quantified. GM6001, ML141 and EHT1864 treatment resulted in similar morphology patterns compared with DMSO control in normal collagen. In thick collagen, GM6001, ML141 and EHT1864 treatment also showed similar morphology but still different from the morphology observed in normal collagen conditions (FIG. 4A, FIG. 4B, and FIG. 12A). These findings suggested that cell morphology was not governed by these drug targets, but was rather still more strongly influenced by the collagen architecture. On the other hand, Y27632, CK666, SMIFH2 and NSC23766 treated cells demonstrated distinct morphologies compared with control. Under the same drug treatment, MDA-MB-231 cells displayed similar morphology in normal collagen and thick collagen. This suggested that the driving force in these conditions was the drug treatment instead of collagen architecture. The graphic illustration of each morphological index can be found in FIG. 12B. Normalized sphericity is shown in FIG. 12C, and image segmentation performance is demonstrated in FIG. 12D.

Notably, each of the observed patterns is directly linked with the cell function directly affected by the inhibitor. Y27632 is a potent and selective ROCK inhibitor. ROCK mediates actomyosin-based cell contractility via regulating retrograde flow of actin monomers from protruding structures (Vicente-Manzanares et al., Nature Reviews. Molecular Cell Biology 2009, 10, 778-790). Inhibition of ROCK in these cultures led to extremely long cell protrusions, as shown in FIG. 4B. Interestingly, cell shape in normal collagen was usually round whereas the cells in thick collagen were slightly elliptical, as indicated by the round/elliptical dotted circle in FIG. 4B. This suggested that the alignment of the cell body in response to collagen architecture may not be entirely abolished with ROCK inhibition.

Nucleation inhibitors triggered distinct morphologies in these collagen cultures. CK666 inhibits function of ARP2/3 whereas SMIFH2 inhibits formins. ARP2/3 often initializes actin fiber branching and is closely associated with lamellipodia. Upon ARP2/3 inhibition, MDA-MB-231 cells, in both normal collagen and thick collagen, were elongated and stretched. This suggested that, in the absence of ARP2/3, formins-driven actin networks may be disrupted and may lead to the elongation of the cell body. However, SMIFH2 treated cells were round and lacked cell peripheral fluctuations (indicated by red arrows on FIG. 4B). This suggested that formins may play a predominant role in cell migration in 3D collagen. NSC23766 shows similar effects as SMIFH2 (FIG. 4B). However, NSC23766- treated cells were smaller than most other drug conditions. This may be linked to NSC23766-induced proliferation arrest, as previously shown in chronic lymphocytic leukemia cells (Hofbauer et al., Blood, 2014, 123, 2181-2188). In addition, SMIFH2-treated cells lacked F-actin accumulation on the cell periphery, clearly visible in NSC23766-treated cells (FIG. 4B, red arrows). NSC23766 and EHT1864 were both Rac1 inhibitors but generated distinct morphological responses in MDA-MB-231 cells in these cultures.

Further quantification of MDA-MB-231 cells morphology agreed with representative images (FIG. 4C, FIG. 4D). In conditions governed by collagen architecture, namely GM6001/ML141/EHT1864/DMSO, the cell population demonstrated almost identical distributions as shown in histograms of each morphology index (FIG. 4C, red dotted arrows).

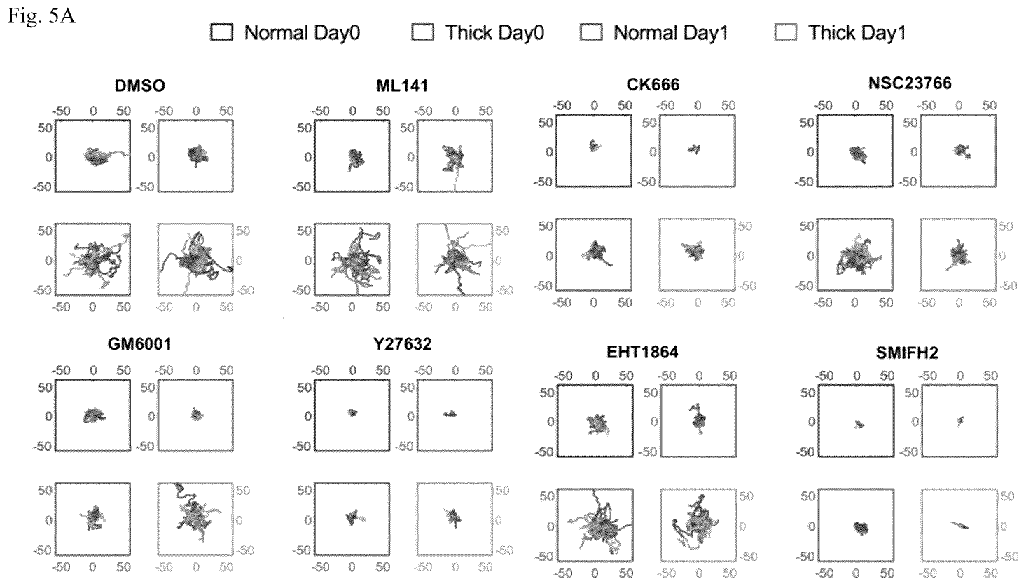

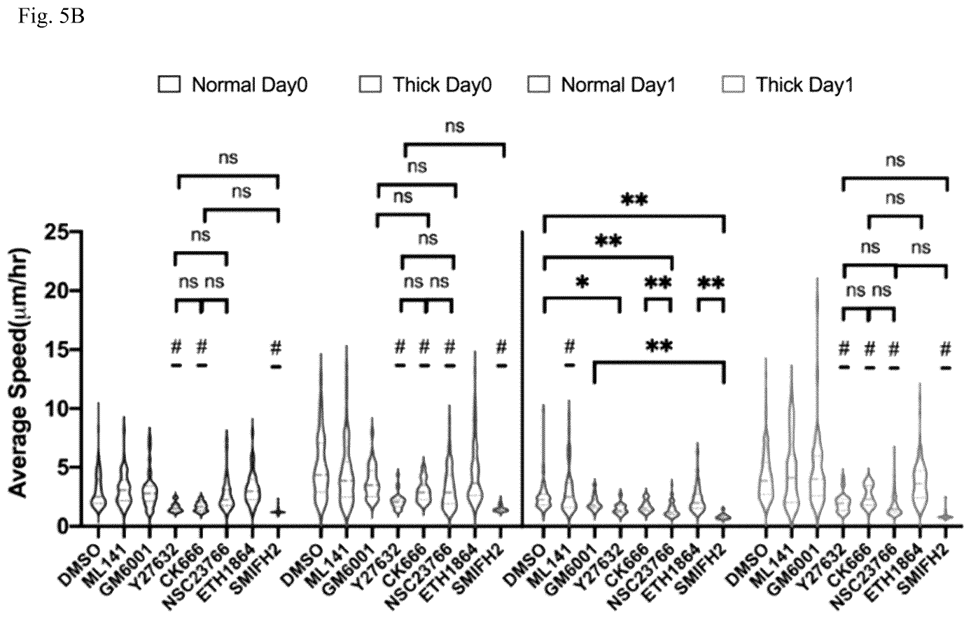

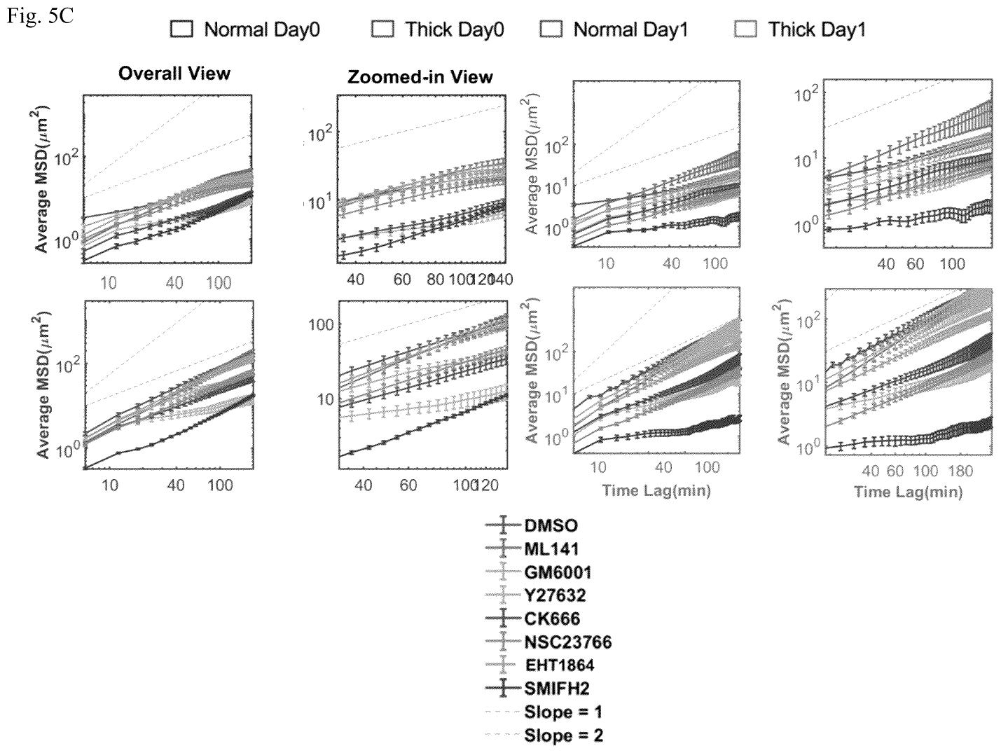

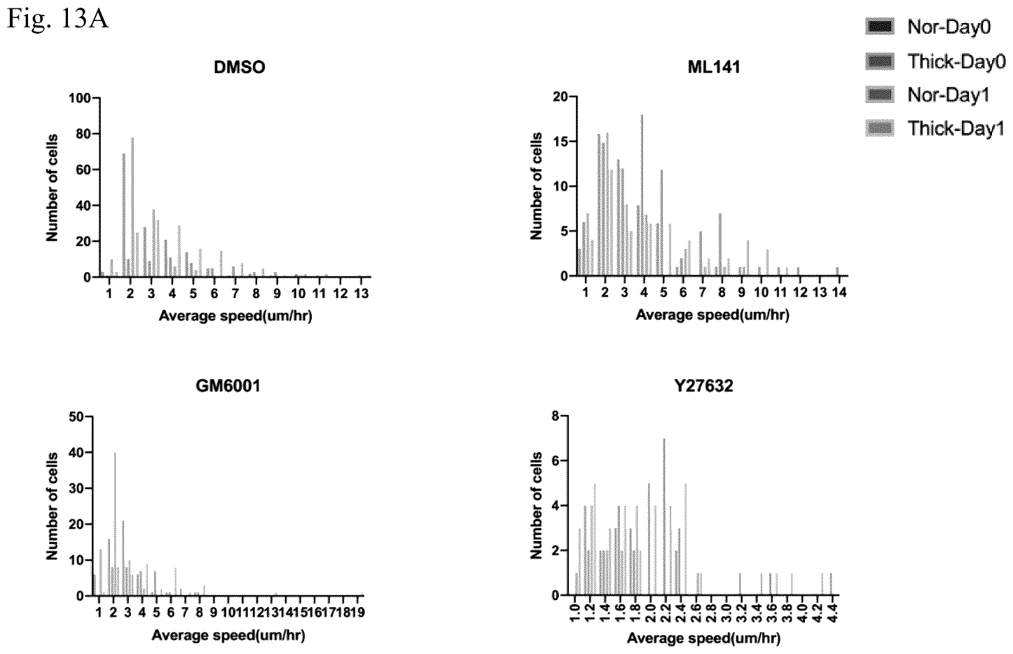

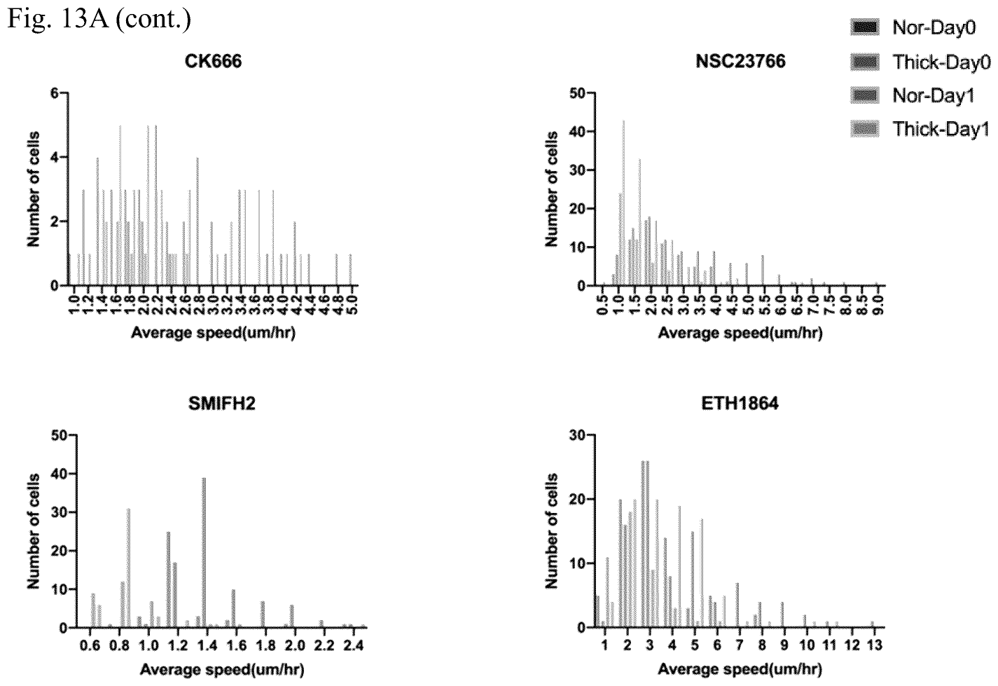

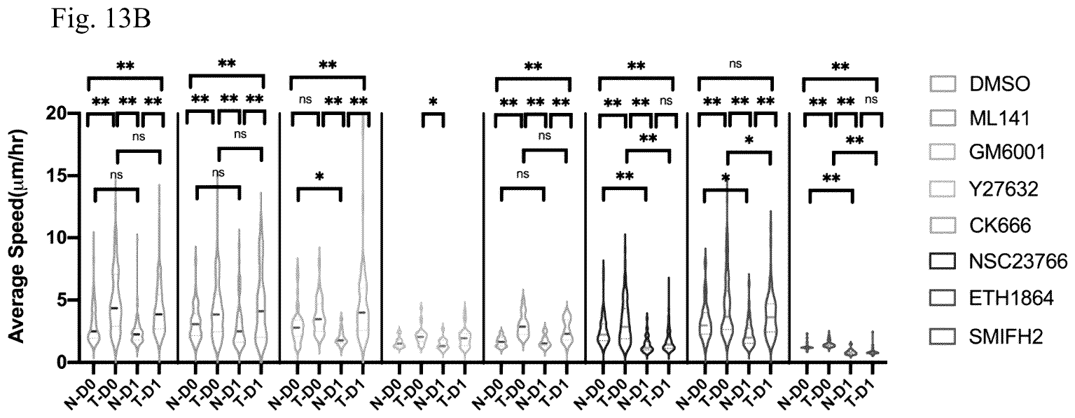

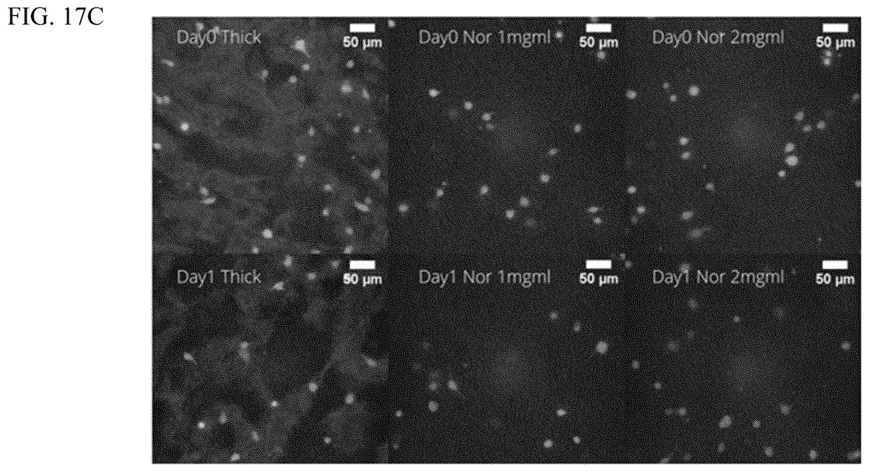

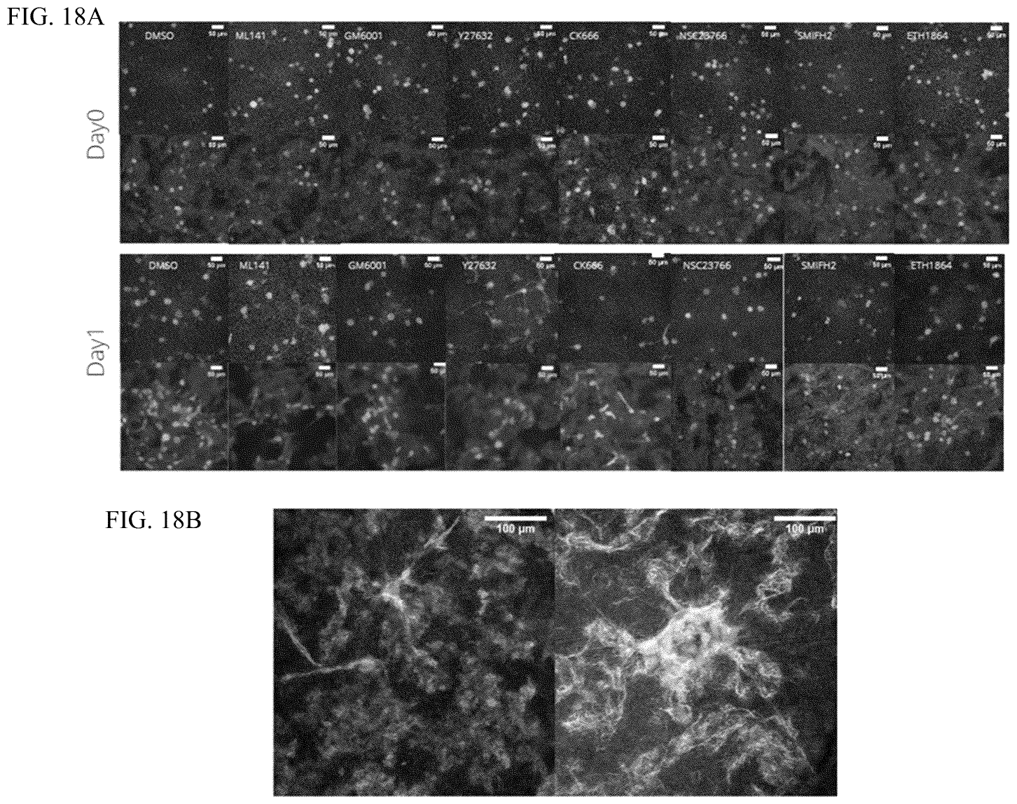

Apart from morphology signatures, cell migration was also tracked (FIG. 5A-C, FIG. 13A-B and FIG. 18A). First, GM6001 treatment failed to inhibit MDA-MB-231 cell migration. GM6001-treated MDA-MB-231 cells in thick collagen migrated as actively as the DMSO control, both increased compared to cells in normal collagen. This suggested that migration of tumor cells in thick collagen was MMP-independent. It was also noticed that Y27632, NSC23766 and SMIFH2 significantly reduced MDA-MB-231 cell migration. This suggested that actomyosin-based contractility by RhoA/ROCK and formin-based actin nucleation were required for cell migration in thick collagen. However, the interpretation of NSC23766 was confounded by the poor performance of another Rac1 inhibitor ML141. NSC23766 binds directly to Rac1 and blocks the interaction between GEF Trio or Tiam (Gao et al., Proceedings Of The National Academy Of Sciences Of The United States Of America, 2004, 101, 7618-7623). EHT1864 binds to Rac1 but it is an allosteric inhibitor for guanine association. EHT1864 can inhibit PDGF-induced lamellipodia formation, but lysophosphatidic acid or bradykinin can still trigger cytoskeletal rearrangement through RhoA and Cdc42 respectively (Shutes et al., The Journal of Biological Chemistry, 2007, 282, 35666-35678). This suggested that the thick collagen architecture may override the effect of EHT1864. On the other hand, ML141, one of the Cdc42 inhibitors, did not show significant inhibition. There are no studies of ML141 on MDA-MB-231 cells, but it has been shown that human pancreatic cancer cells PANC-1 moved faster in aligned matrix fiber regardless of ML141 treatment, whereas NSC23766 inhibited cell movement, regardless of collagen fiber orientation (Wang et al., Nanoscale 2020, 12, 3183-3193), which correlates with these findings. Interestingly, while ML141 does not inhibit migration of MDA-MB-231 cells or pancreatic cancer cells, in 2D conditions it does inhibit ovarian cancer cell lines such as OVCA429 or SKOV3ip, in a dose-dependent manner. The differential preference between MDA-MB-231 cells for a stiff substrate and metastatic ovarian cancer cells (MOCC) for compliant substrate for migration (McGrail et al., Journal of Cell Science 2014, 127, 2621-2626); McGrail et al., Physical Biology 2015, 12, 026001) may be dictated by Rac1 and ML141, respectively.

To conclude, these drug assays together indicate that for highly metastatic MDA-MB-231 cells to migrate in thick collagen, ROCK mediated cell contractility is in the center of tumor cell dissemination. In addition, the role of formins based on SMIFH2 needs to be explored further, since SMIFH2 has been recently reported to have off-target effects on several other non-muscle myosins (Nishimura et al., Journal of Cell Science, 2021, 134). In contrast to the MDA-MB-231 cell line, MCF10A is an immortalized noncancerous breast epithelial cell line. MCF10As lack the potential to form tumors and metastasize in nude mice (Cowell et al., Cancer Genetics and Cytogenetics, 2005, 163, 23-29). When cultured in 3D reconstituted basement membrane, MCF10As undergo differentiation and growth arrest and develop into acinar structures that recapitulate many features of normal breast epithelial cells.

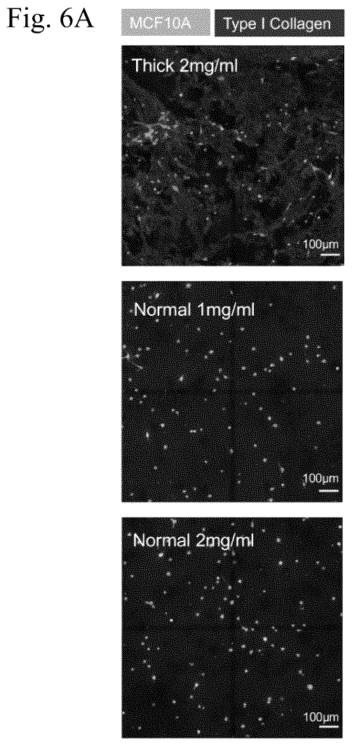

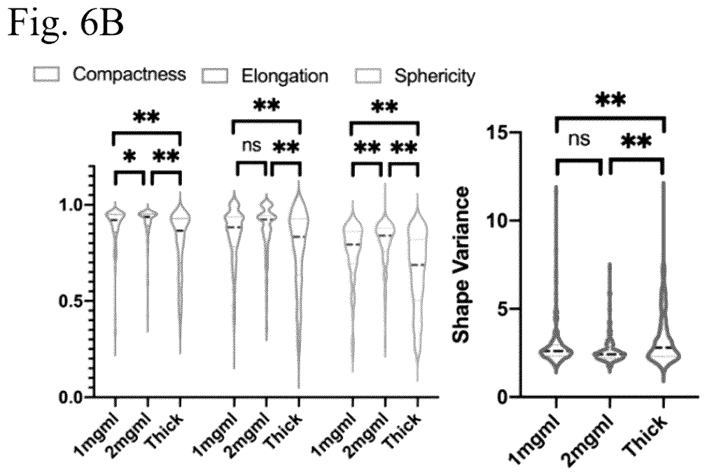

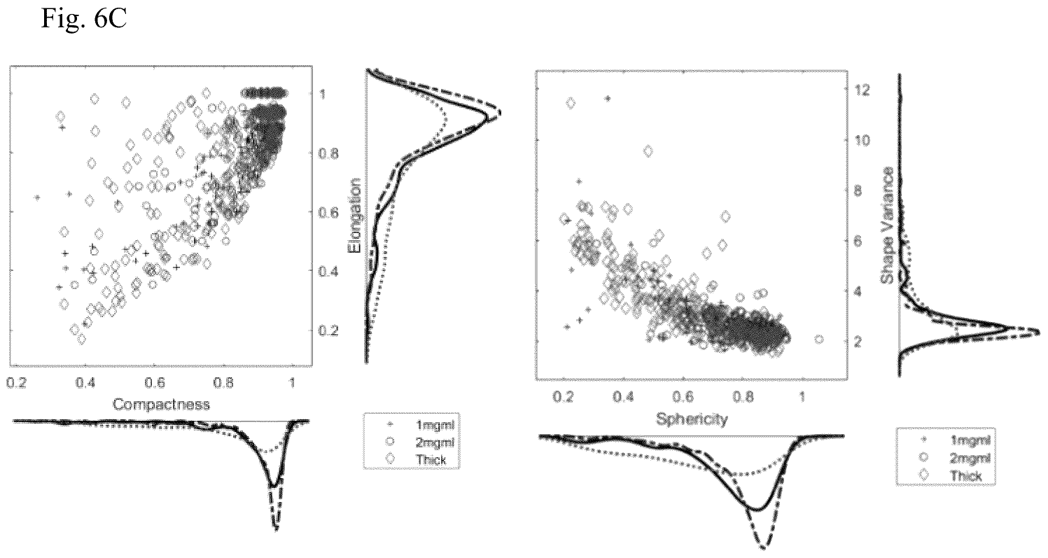

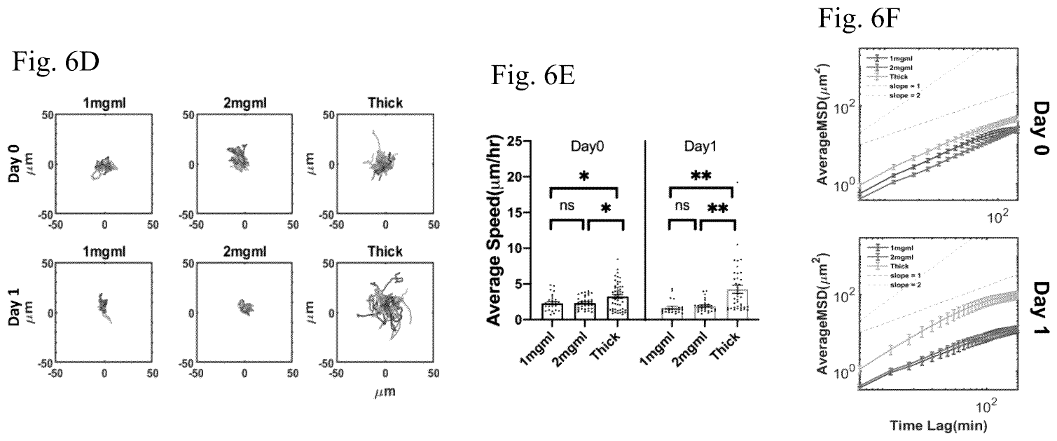

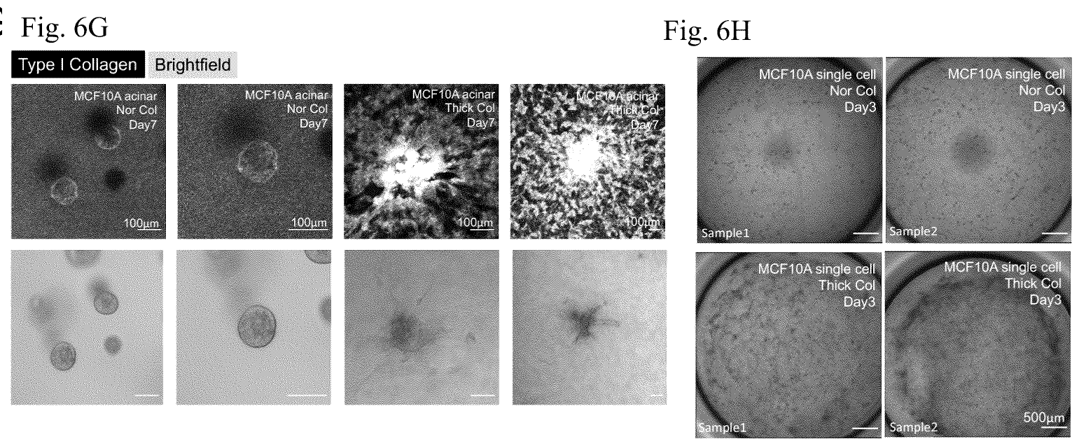

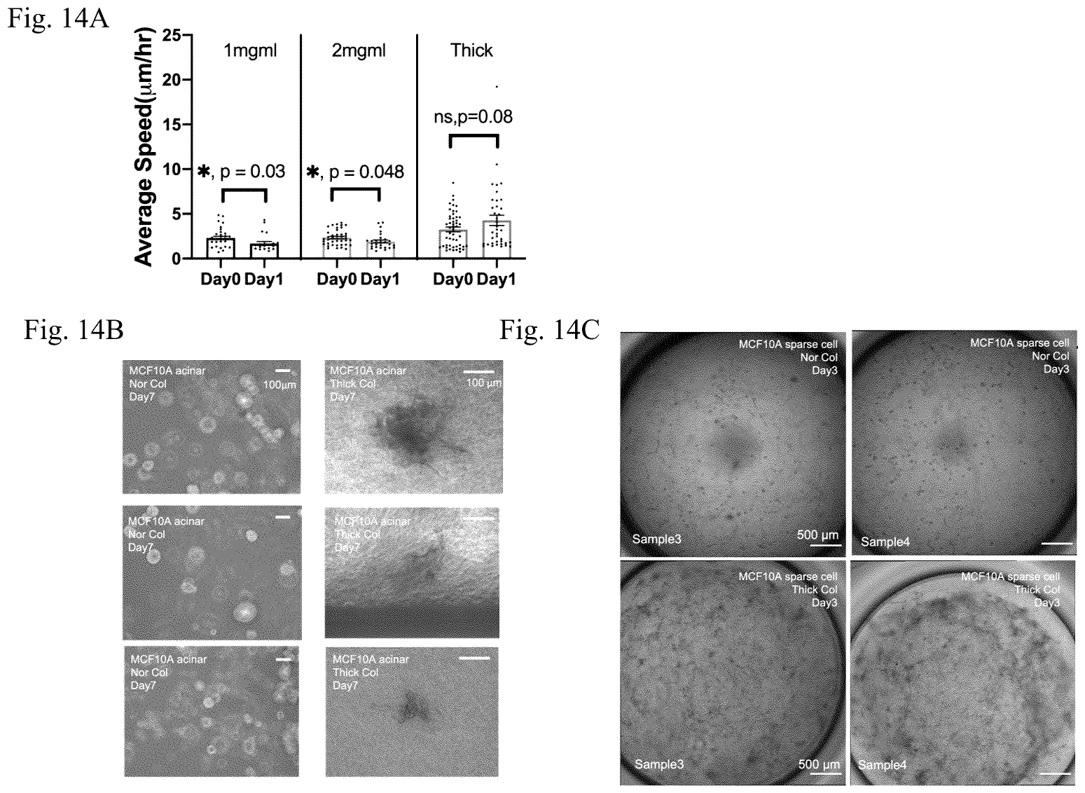

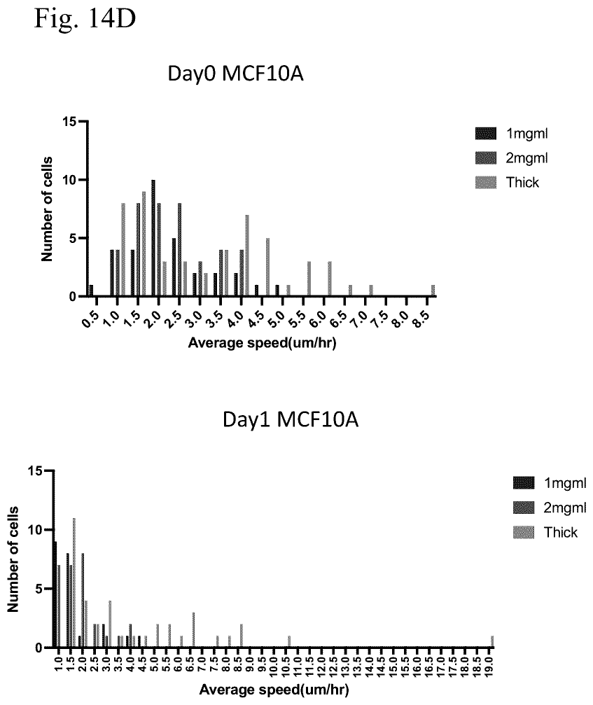

Thick collagen induced different responses from MCF10A single cells compared with both 1 mg/ml or 2 mg/ml normal collagen, but not as dramatic as seen in MDA-MB-231 cells (FIG. 6A) A higher number of MCF10A cells in thick collagen were elongated and less spherical and compact, acquiring a mesenchymal-like morphology (FIG. 6B). A contrast with the irregular protrusion patterns of MDA-MB-231 cells induced by thick collagen architecture was observed, as indicated by the shape variance distribution in FIG. 1B, FIG. 3E-F, FIG. 4B DMSO condition. MCF10A single cells only transitioned from round to more elliptical without producing any small finger-like protrusions, as indicated by the highly concentrated distribution of shape variance in FIG. 6B and FIG. 6C. Also, MCF10A cells were sensitive to thick collagen architecture and influenced by collagen density (FIG. 6C). Consistent with the morphological transition, MCF10A cells had increased migration in thick collagen, as shown in FIG. 6D and SI FIG. 6D. MCF10A cells migrated significantly faster (FIG. 6E, FIG. 14A) and more persistently (FIG. 6F) in thick collagen than 1 mg/ml or 2 mg/ml normal collagen.

Thick collagen also triggered morphological changes of MCF10A acini. Protrusive branches appeared from the acinus and pulled the collagen patches nearby substantially (FIG. 6G). MCF10A acini seeded in normal collagen showed little of these collagen-pulling. Additional data can be seen at FIG. 14B. It has been previously documented that MCF10A acini generate protrusive cell strands in response to mechanical forces (Shi et al., Proceedings Of The National Academy Of Sciences Of The United States Of America, 2014, 111, 658-663).

The collagen-pulling was not limited to MCF10A acini but also observed in single cells. In overview tile scan imaging (FIG. 6H), morphological changes were universal and occurred across the entire well, accompanying the collapse and detachment of the thick collagen bundles from the vertical wall of culture wells. Additional data can be seen at FIG. 14C.

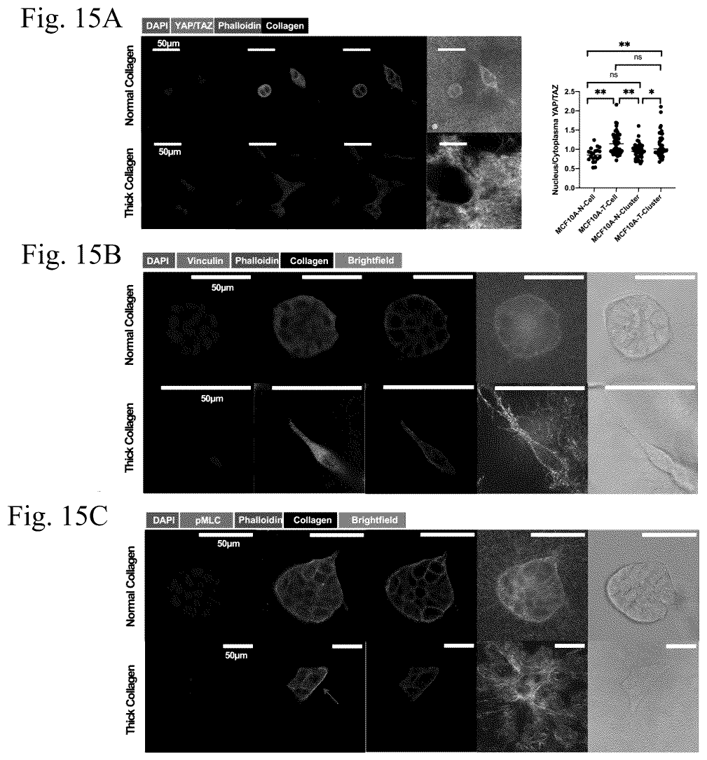

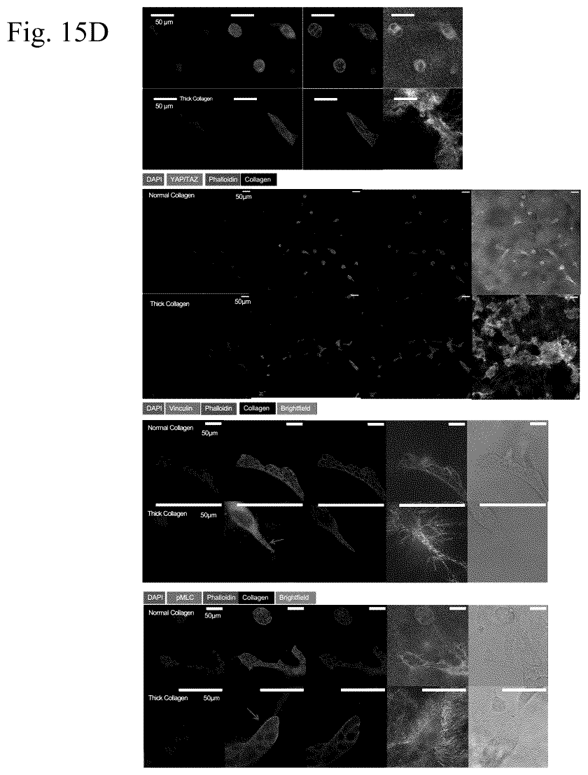

To investigate the molecular signatures induced by thick collagen in MCF10A cells, immunofluorescence imaging of YAP/TAZ, vinculin, and phosphorylated myosin light chain (pMLC) was collected. YAP/TAZ is a sensitive mechanosensor, and its relative nuclear/cytoplasmic distribution is regulated by substrate rigidity (Dupont et al., Nature, 2011, 474, 179-183). YAP/TAZ accumulates in the nucleus when a cell stretches out on a substrate of high rigidity but localizes in the cytoplasm when cells are in confinement or interacting with softer substrates. As MCF10A cells often clustered in collagen after three days after gel embedding, MCF10A clusters were separated from MCF10A single cells in YAP/TAZ measurement. YAP/TAZ quantification revealed that both MCF10A single cells and MCF10A clusters had a higher nucleus/cytoplasm ratio in thick collagen versus normal collagen (FIG. 7A, FIG. 15A-B). This was consistent with the previous observation of highly stretched MCF10A clusters in thick collagen. Thick collagen architecture triggered stretching and elongation of MCF10A cells and redistribution of YAP/TAZ.

Vinculin is a molecular marker for both intercellular and cell-ECM adhesions (Bays & DeMali, Cellular and Molecular Life Sciences: CMLS, 2017, 74, 2999-3009). In the protrusive strands from MCF10A cell clusters, vinculin (FIG. 7B, red arrows, FIG. 15C) colocalized with strained collagen bundles. This suggested that the collagen pulling observed in FIG. 6G and FIG. 6H may be linked to protrusive structures with strong adhesions.

pMLC is a direct marker for actomyosin based contractility. Data in both FIG. 7C and FIG. 15D demonstrated that, in thick collagen where large gel patch contraction happens, increased pMLC can be observed. This indicated that the collagen-pulling observed in FIG. 6G and FIG. 6H was linked to cell contractility.

In this work a fast and reproducible method to modulate collagen architecture was introduced and created a type of collagen scaffold highly similar to in vivo stromal architecture heterogeneity (FIGS. 10A, 10B, and 10D). As the tumor microenvironment often features wavy and bundled collagen fibers, embedded human cells were embedded into the system. Regardless of their initial invasive potential, increased migration was observed in the breast cancer cell line MDA-MB-231 and the immortalized breast cell line MCF10A in thick collagen scaffolds. In thick collagen, MDA-MB-231 cell dissemination was significantly augmented in co-culture with normal human lung fibroblasts (FIG. 16, FIG. 18B). Fast migration of MDA-MB-231 cells relied on ROCK-mediated cell contractility and forming-based protrusive structures but is MMP-independent. Characterization of both MCF10A single cells and MCF10A acini demonstrated differential behavior in thick collagen, compared with normal collagen. These findings agree with observations of cells on wavy ECM substrates, which highlight the role of cell contractility ((Fischer et al., Proceedings of The National Academy of Sciences Of The United States Of America 2021).

There have been many previously reported different ways of modifying collagen architecture for 3D cultures. However, compared with previous work, these collagen scaffolds demonstrated better global resemblance to in vivo ECM architecture (FIGS. 10A and 10B). This architecture was generated in reconstituted collagen gels by applying mechanical agitation during fibrillogenesis and gelation. Thus, this method opens a new way of collagen architecture modification by integrating mechanical shearing during in vitro fibrillogenesis.

The instantly presented thick collagen may serve as a better platform to examine and compare cell migration behaviors in 3D, as this type of collagen architecture has decoupled the confounding effect of physical restraints from other factors. Physical limits or steric restraints of collagen greatly impact cell behavior. Most previous reports of tumor cell migration in 3D collagen are based on the normal collagen scaffold, which is strongly impacted by matrix proteolysis (Zaman et al., PNAS 2006, 103, 10889-10894; Sabeh et al., The Journal of Cell Biology, 2004, 167, 769-781). These studies suggest the potential of MMP inhibition as a strategy against invasion. The instantly presented thick collagen suggests an adjusted view of MMP inhibitors that they may be ineffective in heterogeneous and clustered collagen environments (similar to the thick collagen gels presented herein). In addition, the results agree with intravital imaging work (Gligorijevic et al., PLOS Biology, 2014, 12, e1001995) that the migration of breast tumor cells in vivo is MMP-independent and may provide additional indications for the reasons of the failure of MMP inhibitors in clinical trials (Fingleton, The Cancer Degradome, 759-785). Without physical restraints, thick collagen can help further stratify drug performance in in vivo-like microenvironments. For example, in normal collagen, GM6001/ML141/SMIFH2/NSC23766/EHT1864 appear to exert the same effects on cell morphology (all cells are round). However, MDA-MB- 231s in thick collagen fall into two subgroups: GM6001/ML141/EHT1864 versus SMIFH2/NSC23766 (FIG. 4B). Therefore, thick collagen bundles have the potential to be applied in drug screening targeting cell phenotypes in more complex microenvironments.

Thick collagen may serve as a better platform to examine and compare cell migration behaviors in 3D, as this type of collagen architecture has decoupled the confounding effect of physical restraints from other factors. Physical limits or steric restraints (Mosier et al., Biophysical Journal, 2019, 117, 1692-1701); Guzman et al., Biomaterials, 2014, 35, 6954-6963) of collagen greatly impact cell behavior. Most previous reports of tumor cell migration in 3D collagen were based on the normal collagen scaffold, which was strongly impacted by matrix proteolysis (Wolf et al., The Journal Of Cell Biology, 2013, 201, 1069-1084; Zaman et al., Proceedings Of The National Academy Of Sciences Of The United States Of America, 2006, 103, 10889-10894; Sabeh et al., The Journal Of Cell Biology, 2004, 167, 769-781), suggesting MMP inhibition as a strategy against invasion. However, thick collagen suggested an adjusted view of MMP inhibitors since they may be ineffective in heterogeneous and clustered collagen environments such as thick collagen gels. In addition, the results agreed with intravital imaging work (Gligorijevic et al., PLoS Biology, 2014, 12, e1001995) that the migration of breast tumor cells in vivo is MMP-independent and may provide additional indications for the reasons for the failure of MMP inhibitors in clinical trials (in The Cancer Degradome 759-785). Without physical restraints, thick collagen could help further stratify drug performance in in vivo-like microenvironments. For example, in normal collagen, GM6001/ML141/SMIFH2/NSC23766/EHT1864 appeared to exert the same effects on cell morphology. However, MDA-MB-231 cells in thick collagen fell into two subgroups: GM6001/ML141/EHT1864 response versus SMIFH2/NSC23766 response (FIG. 4B). Altogether, thick collagen bundles have the potential to be applied in drug screening for targeting cell phenotypes in more complex microenvironments.

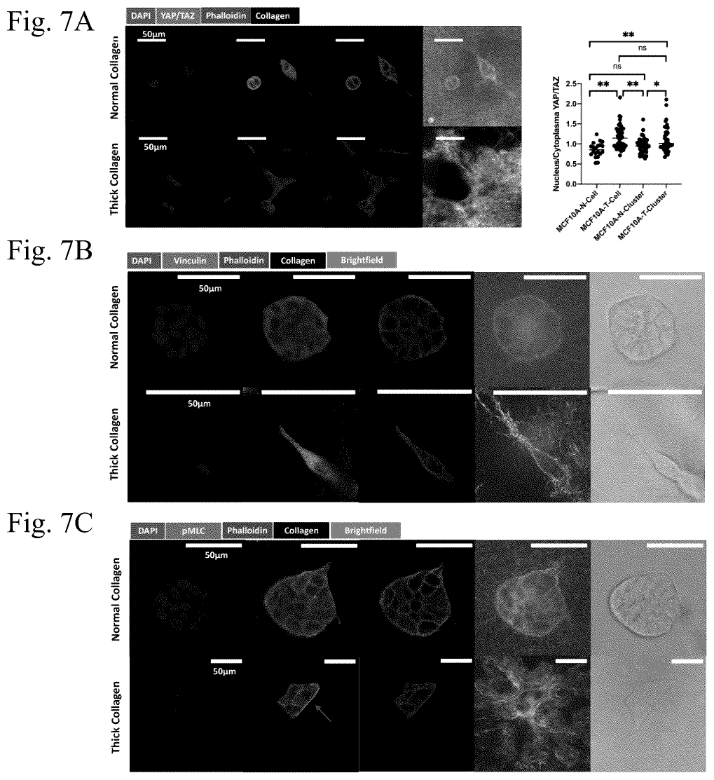

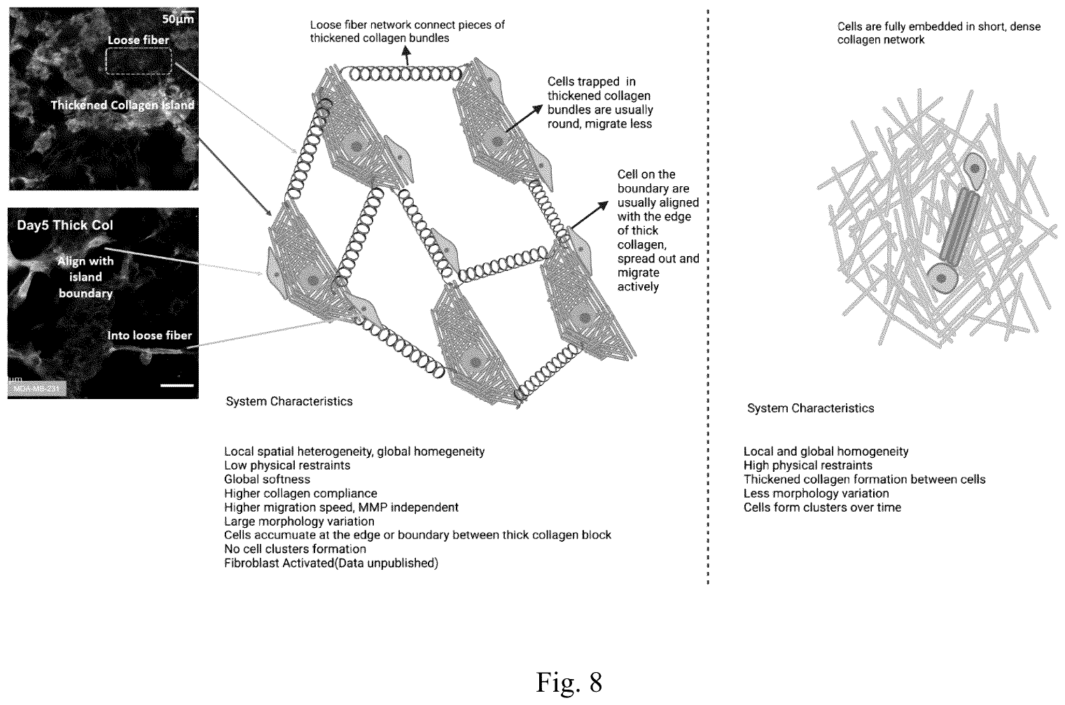

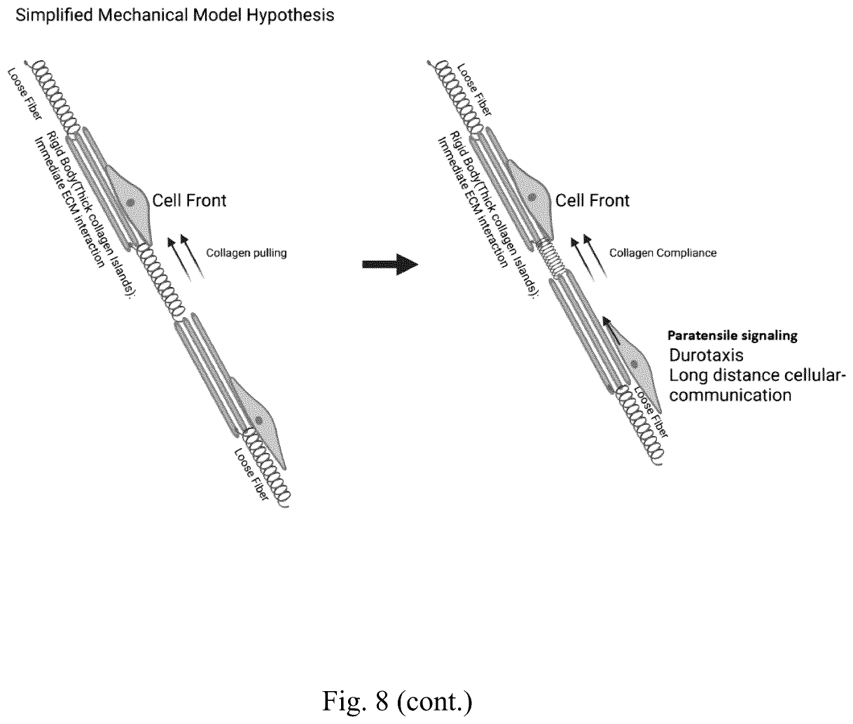

Further, this type of collagen can be easily integrated into coculture assays as with normal collagen. The unique architecture and mechanical features of this thick collagen introduce a different overall picture of the mechanical landscape that scaffolds cells in culture. This method captures the spatial heterogeneity in the tumor microenvironment. These findings echoed one intravital imaging study of breast cancer (Gligorijevic et al., PLoS Biology, 2014, 12, e1001995) which identified two subpopulations of tumor cell phenotypes, and their multiparameter classification links the driving factor of these two subpopulations to their relative locations in the TME. This study also supported the importance of spatial heterogeneity of TME. The overall features of this thick collagen system are summarized in FIG. 8. Existence of paratensile signaling in thick collagen networks is also proposed in Fig. d8, which may account for the increased MDA-MB-231 migration in the presence of NHLF in thick collagen (FIG. 16). A current limitation in this system was a lack of molecular and cellular complexity. Importantly, thick collagen scaffolds were solely composed of collagen I and do not include other important ECM proteins such as hyaluronic acid, laminin, or fibronectin. In addition, these systems lacked other important cells which contribute to the TME such as vascular endothelial cells and immune cells. Other studies have created microtissue cancer scaffolds containing many cell types which not only gave rise to tumors with more physiologically relevant cell-cell interactions but also stimulated deposition of other ECM proteins which are correlated with cancer progression (Acta Biomaterialia, 2018, 73, 236-249). Future studies will explore how the addition of other cell types which stimulate or participate in ECM protein deposition can affect cancer cell migration in thick collagen scaffolds.

As thick collagen gels were locally dense but globally soft, they provided a way to separate local scale mechanics from global scale (or mesoscale) mechanics. Mechanical testing implied that the thick collagen behaves as a more fluid-like material. Additionally, previous research has shown that stiffness of collagen increases over collagen concentration (Motte & Kaufman, Biopolymers, 2013, 99, 35-46). From the confocal imaging, thick collagen bundles are of higher density compared with thin collagen networks in between. Taken together, it may be possible to conclude that thick collagen networks have a weaker, more fluid-like background but contain stiff inclusions. Previous studies have shown the importance of scales in mechanical interactions. For example, long-distance matrix-mediated mechanical communication exists (Sunyer et al., Science, 2016, 353, 1157-1161; van Oers et al., PLoS Computational Biology 2014, 10, e1003774; Wang et al., Biophysical Journal 2014, 107, 2592-2603; Pakshir et al., Nature Communications, 2019, 10, 1850; Reinhart-King et al., Biophysical Journal 2008, 95, 6044-6051)), fibroblasts can recruit macrophages from 100-200 micrometers away in fibrillar collagen (Wozniak et al., The Journal Of Cell Biology, 2003, 163, 583-595), floating gel regulates differently compared with anchored gel (Grinnell et al., The Journal Of Biological Chemistry 1999, 274, 918-923; Leong et al., Biochemical And Biophysical Research Communications 2010, 401, 287-292)), with the same surface coating, the thickness of the coating regulates cell behavior, tissue-scale deformations can control wound closure (Sakar et al., Nature Communications, 2016), among others. The creation of mesoscale clustered collagen architectures may provide a solution to these problems. Outside tumor modeling, the thick collagen gels have potential applications in 3D wound modeling, stem cell differentiation, and primary cell cultures.

### Collagen and Collagen Island Architecture

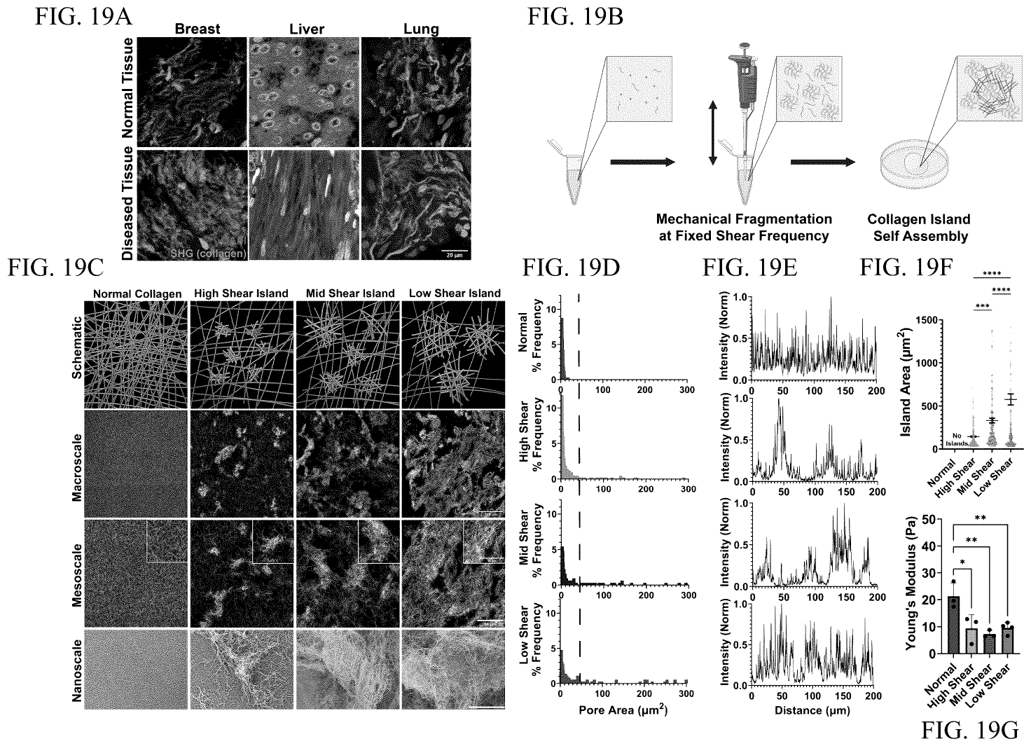

In physiological tissues, ECM topologies and architectures can be diverse, with regions of varying density and stiffnesses (FIG. 19A). These properties are crucial for determining cell function. To recreate the heterogeneous architecture of physiological ECM, a method to introduce inclusions into collagen hydrogels was developed. This process involved mechanical fragmentation during the collagen gelation process at regular intervals to produce collagen “island” inclusions embedded into the hydrogel. By modulating shearing frequency, the amount of shear stress introduced during gelation was altered (FIG. 19B).

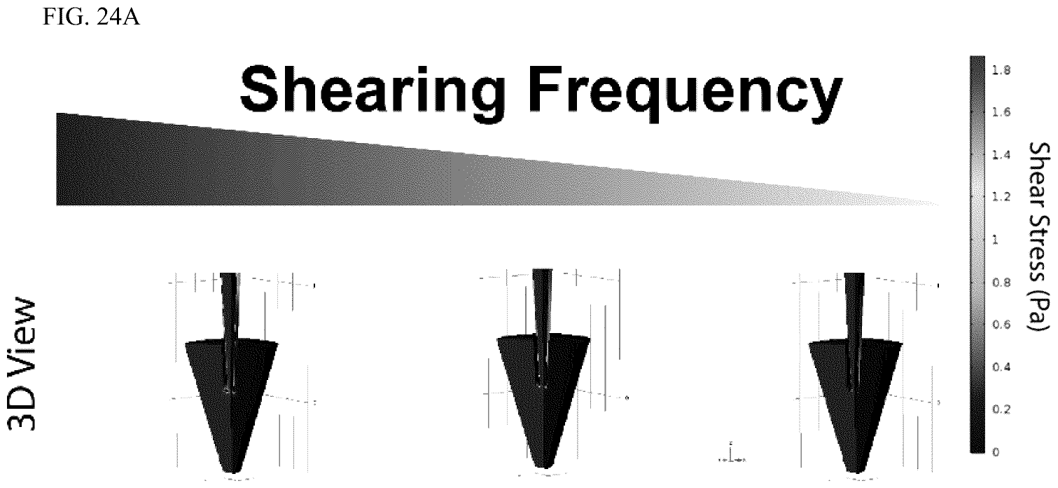

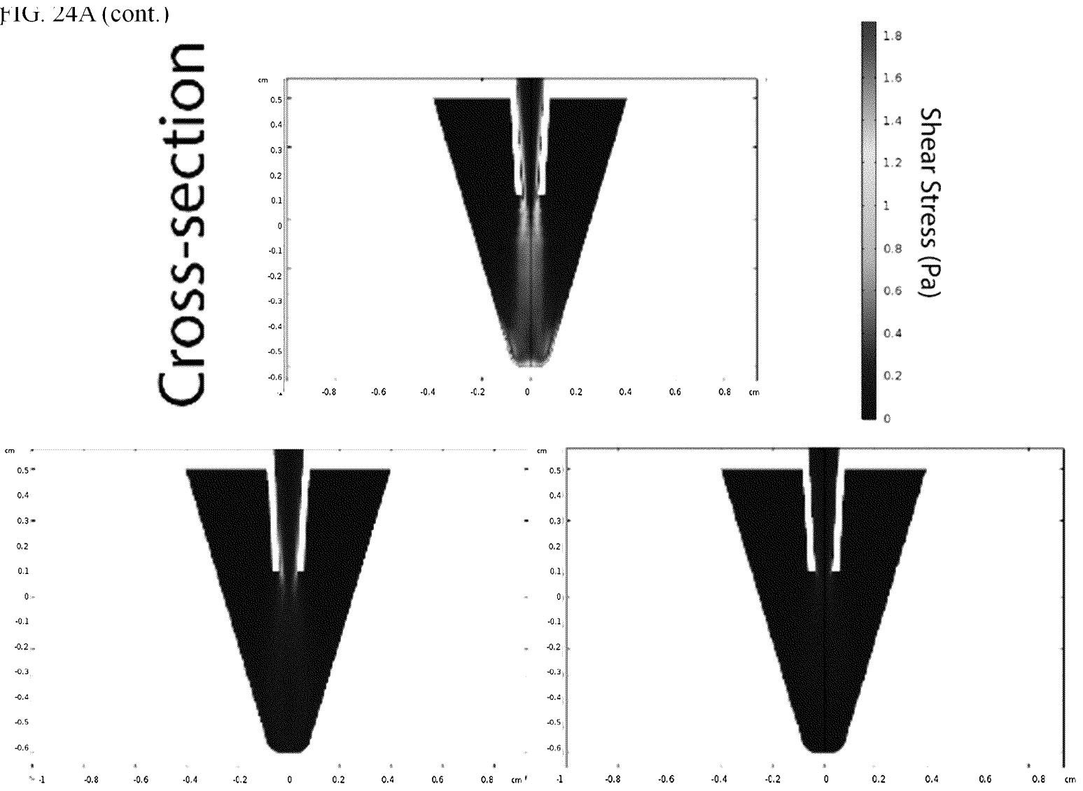

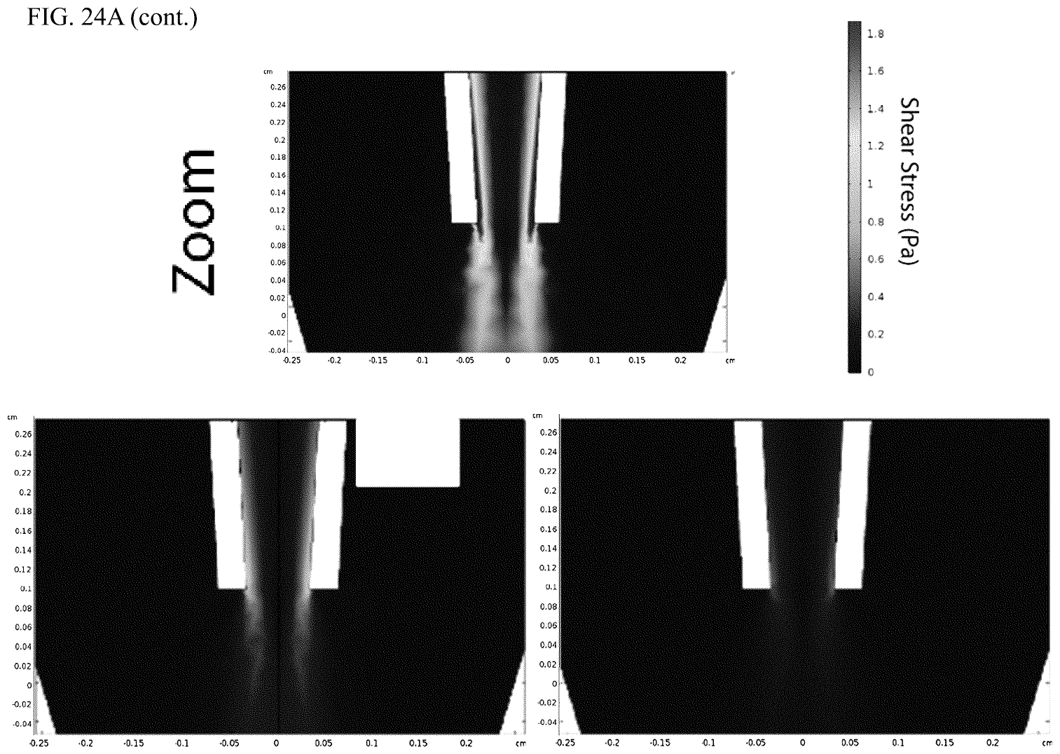

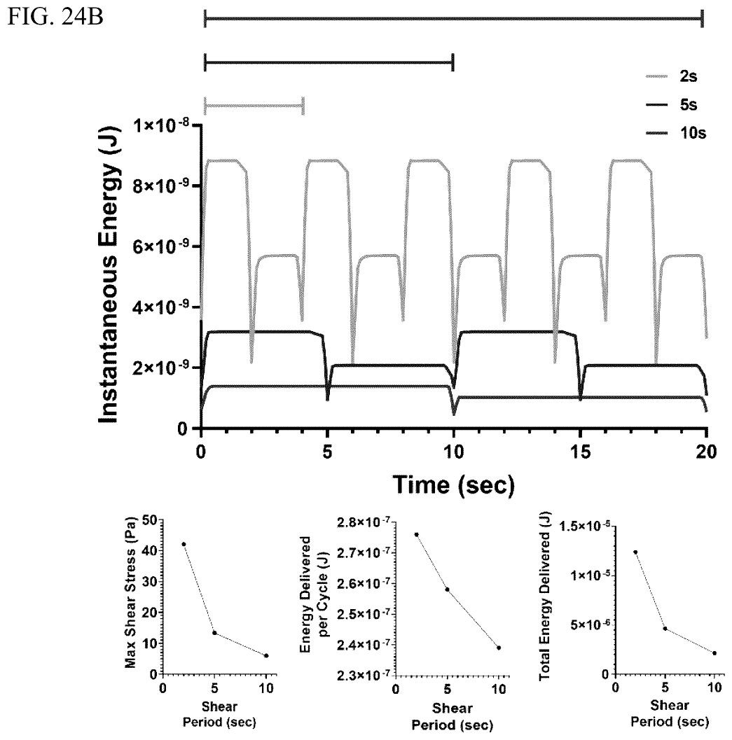

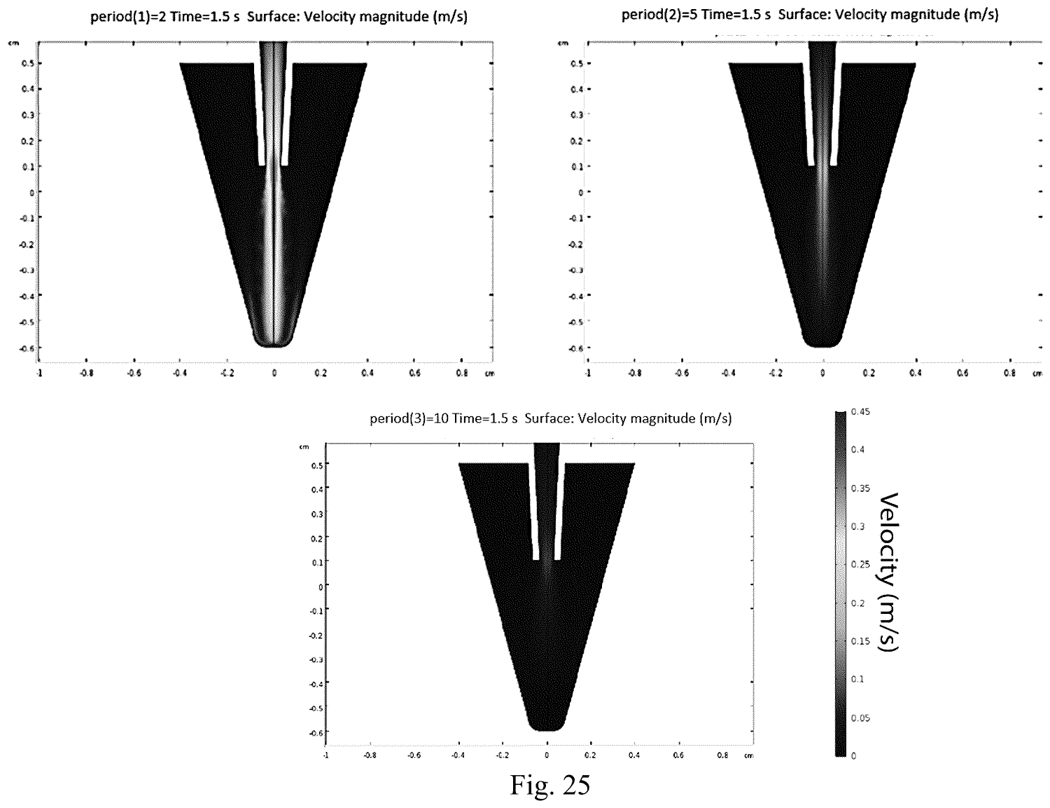

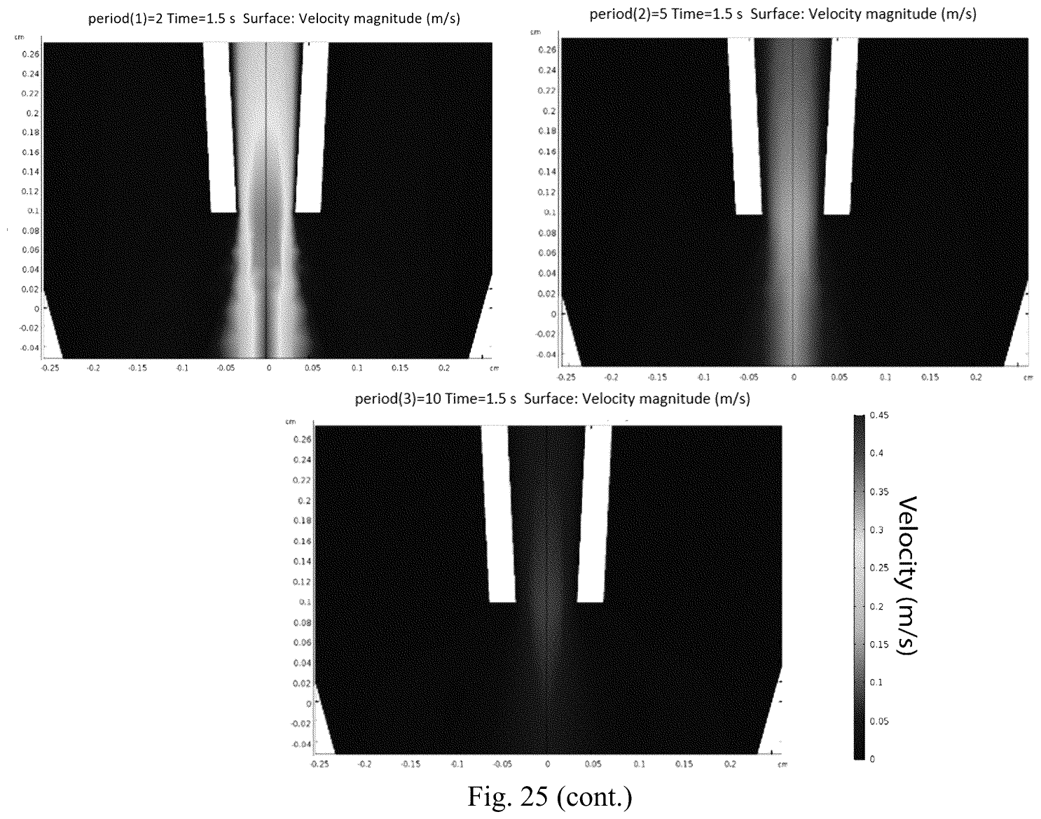

Fluid dynamics simulations revealed how much shear stress and energy are introduced into gels during this process (FIG. 24A). By shearing at a higher frequency, it was observed that more energy and shear stress were introduced into the system (FIGS. 24B and 25). Interestingly, while most of the shear stress being introduced into the system comes from shear induced by the pipette tip, a significant amount of stress developed as the fluid hit the bottom of the centrifuge tube. These simulations allowed for the separation of island gels into distinct architectures based on the shear frequency: a high shear island gel (oscillatory mixing with a period of 2 seconds), a mid-shear island gel (oscillatory mixing with a period of 5 seconds) and low shear island gel (oscillatory mixing with a period of 10 seconds).

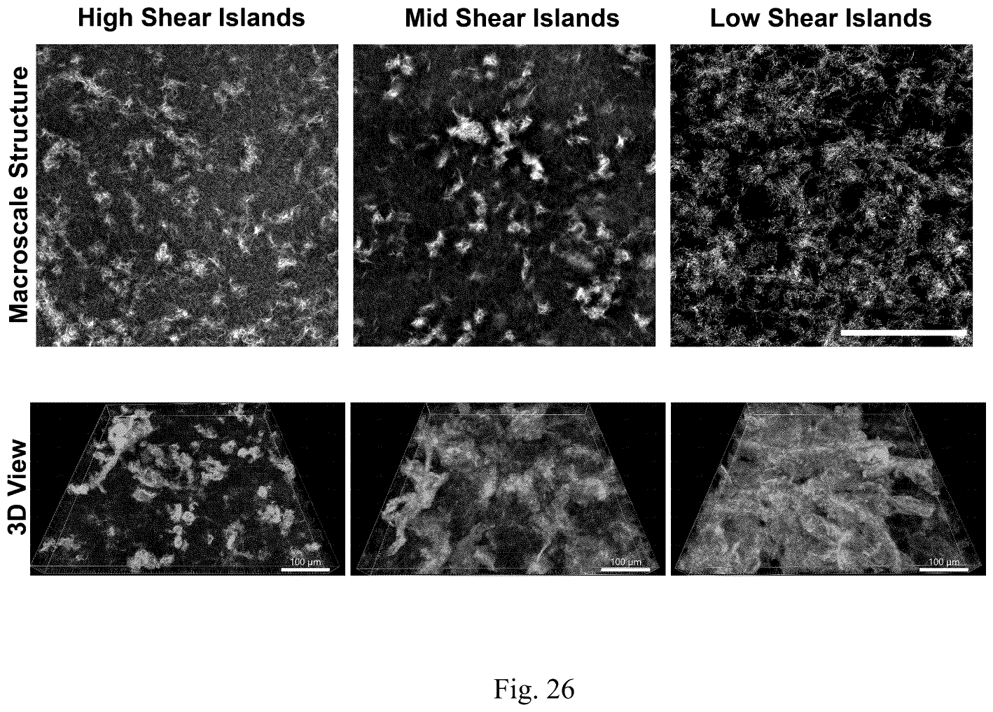

In accordance with simulations, it was found that island size could be reproducibly modulated with 2 second gels having smaller islands and 10 second having larger islands (FIG. 19C). During the mixing stage, collagen gelation has not completed as this is occurring during the transient gelation phase right after collagen neutralization. Thus, nucleation, polymerization, gelation, and agglomeration are all occurring during island gel synthesis. Large scale confocal microscopy and subsequent 3D reconstructions reveal that these island architectures are well distributed throughout the gel (FIG. 26). All gels are a final concentration of 2 mg/ml. The gels were then allowed to sit at room temperature for 6.5 minutes. Finally, the gels were sheared with a p200 pipette tip set to 175 ul at a specified frequency (2 second, 5 second, or 10 second period) for 3 minutes before being plated. All gels are a final concentration of 2 mg/ml. For simplicity, isotropic 2 mg/ml gels with no mechanical perturbation are referred to as normal gels.

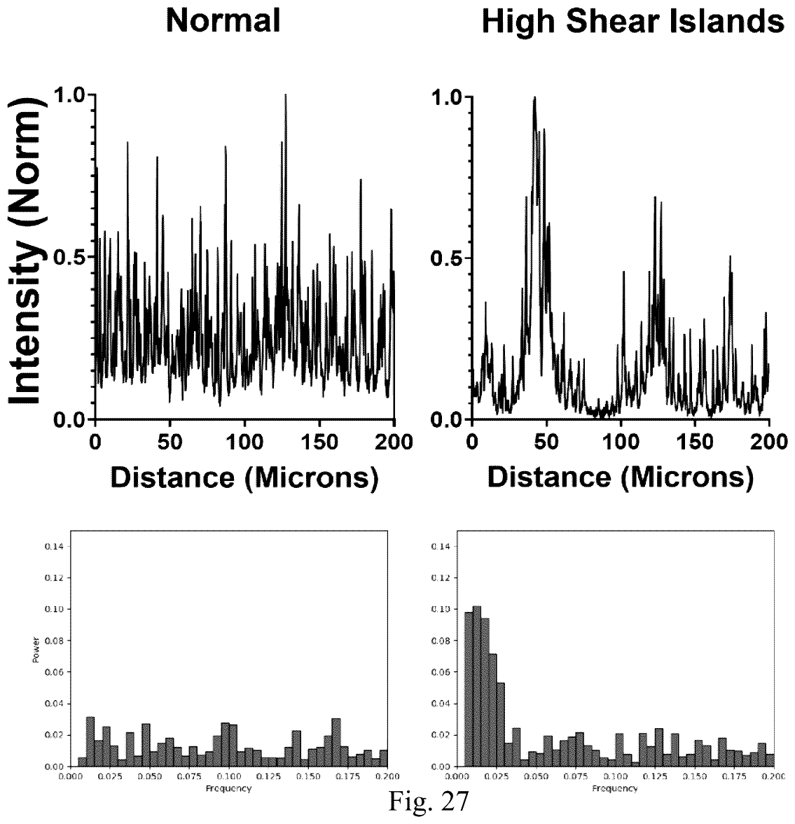

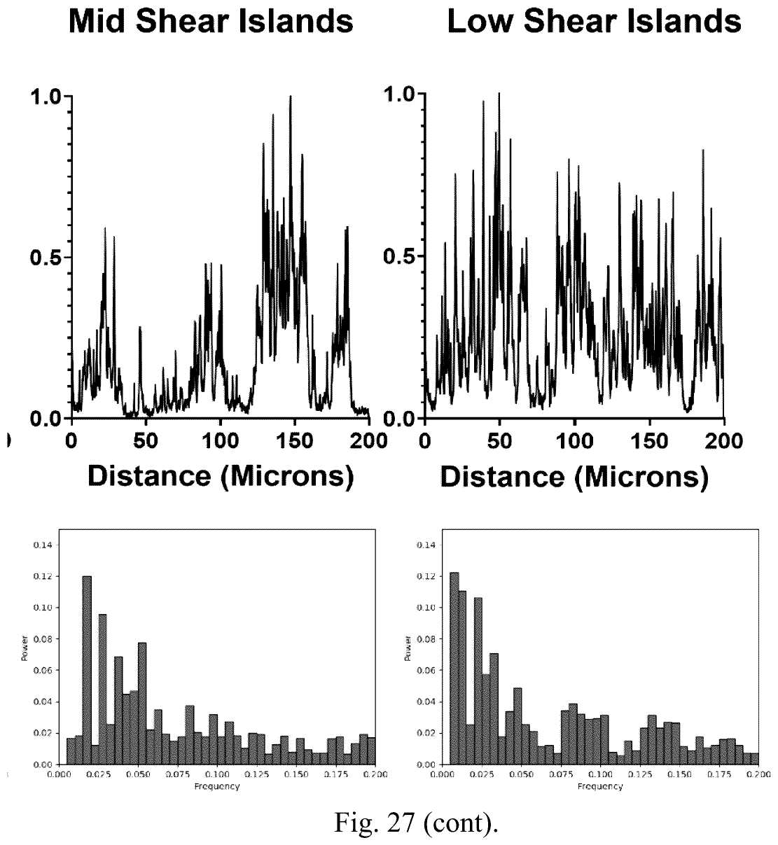

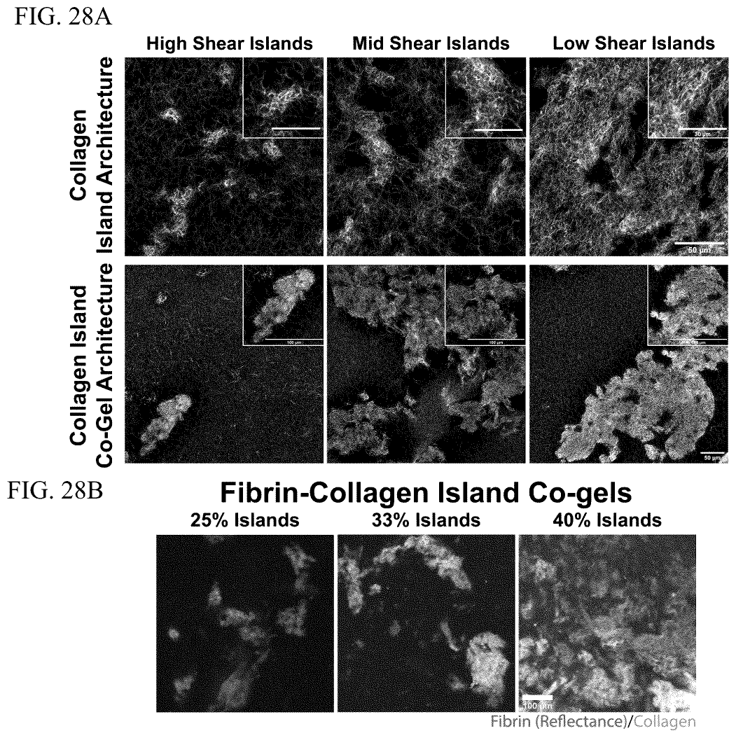

These island architectures and their packing within the gel were observable at the macro, meso and nano scales. Confocal microscopy revealed that increasing shearing frequency caused inhomogeneity in pore size in our gels, indicated by higher occurrence of larger pore areas (FIG. 19D, right side of dotted line). This suggested that, as shear frequency increases, more collagen became a part of the island architecture and the background fibers became more sparse. Furthermore, confocal microscopy revealed that mechanical shearing introduced inhomogeneous architectures into the collagen gel and that increasing shearing frequency decreased the packing fraction of islands (FIG. 19E, FIG. 27). These islands are highly stable, being able to be spun down and resuspended in smaller volumes of collagen or in different biomaterials to tune island packing fraction (FIG. 28). Increasing mechanical fragmentation yielded hydrogels with smaller islands as well as softer bulk stiffness as determined by shear rheometry (FIGS. 19F and 19G). While bulk stiffness is used here, confocal microscopy suggested local differences in stiffness in island gels, with the islands being higher density collagen structures and, therefore stiffer than the background collagen embedded between the islands (Motte, Biopolymers, 2013, 99, 35-46).

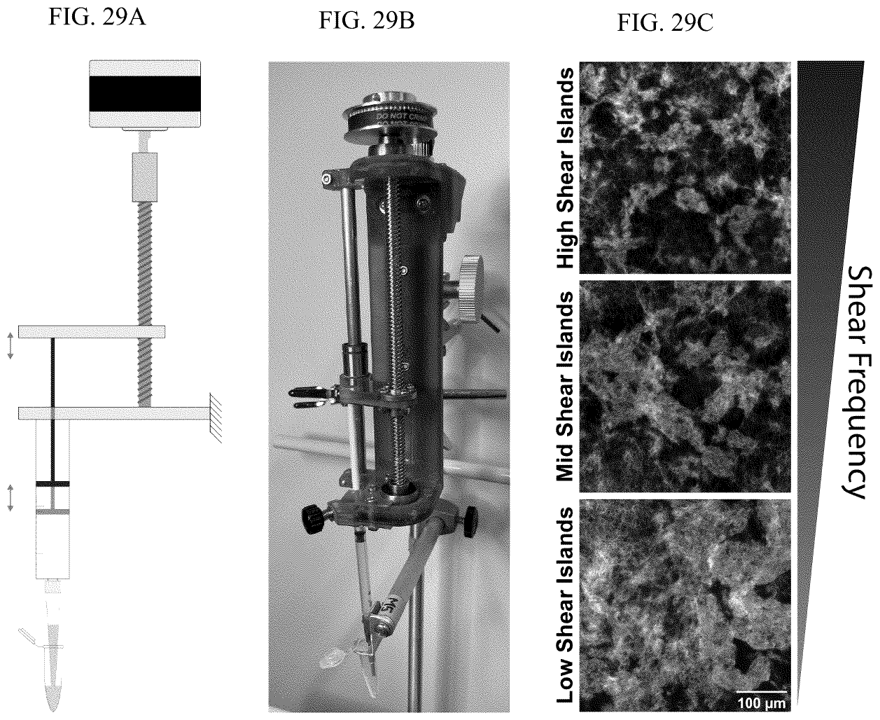

To improve scalability, an automated collagen mixer was developed (FIGS. 29A and 29B). Shear frequencies were able to be inputted and recapitulated collagen island architectures made by hand (FIG. 29C). Future studies can further utilize this tool to scale up and automate island architecture assembly as well as efficiently explore the phase space that underlies island architecture. Together, these data suggested that collagen islands are easily tunable with the capability for modular assembly.

Mechanical properties of tunable collagen islands

Stiffness is only one mechanical property of biomaterials that can affect cell behavior. In particular, collagen can undergo nonlinear strain stiffening in which collagen can increase in stiffness significantly under increasing applied strain. Notably, cells are able to mechanically remodel collagen to sufficiently induce strain stiffening, creating stiffness gradients over hundreds of microns (Winer et al., PloS One 2009, 4, e6382; Wang et al., Biophysical Journal, 2014, 107, 2592-2603; Rudnicki et al., Biophysical Journal, 11-20). Strain stiffening has been shown to contribute to a variety of cell behaviors including durotaxis, focal adhesion development, and higher migratory behavior. In addition, collagen is viscoelastic and can reorganize molecularly over time under applied strain leading to stress relaxation. Viscoelasticity in biomaterials has been implicated in a number of cellular processes including cell spreading, cell migration, and differentiation (Lee et al., Science Advances, 2021, 7; Lee et al., Nature Materials 2017, 16, 1243-1251; Chaudhuri et al., Nature Materials 2016, 15, 326-334; Chrisnandy et al., Nature Materials, 2022, 21, 479-487). While these properties have been studied extensively in the literature, how tissue architecture affects these mechanical properties is poorly understood.

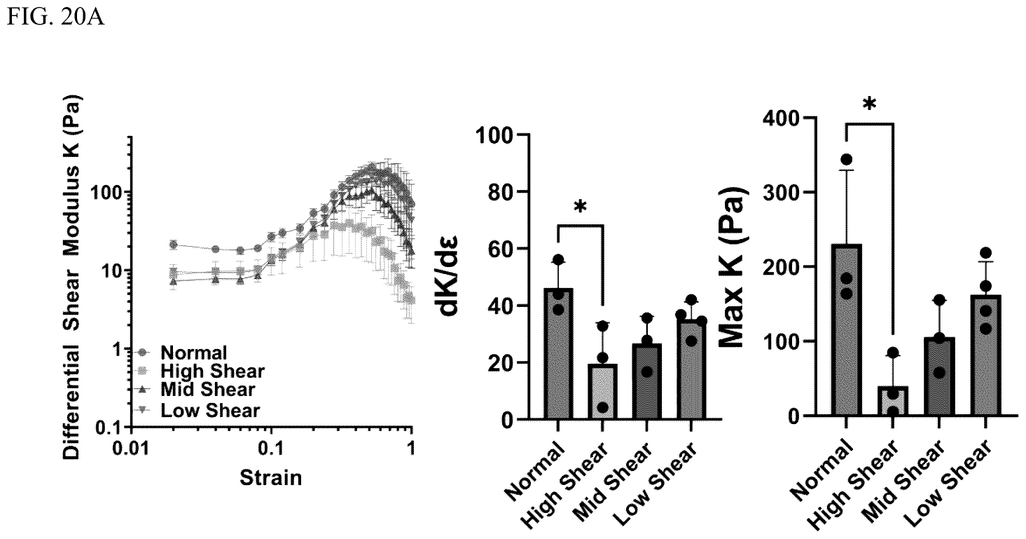

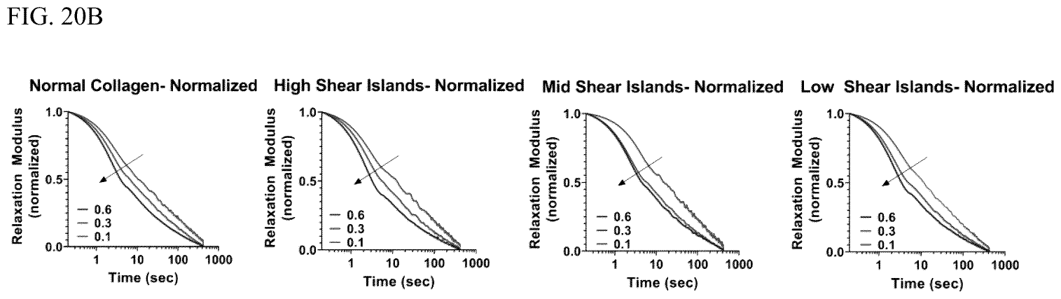

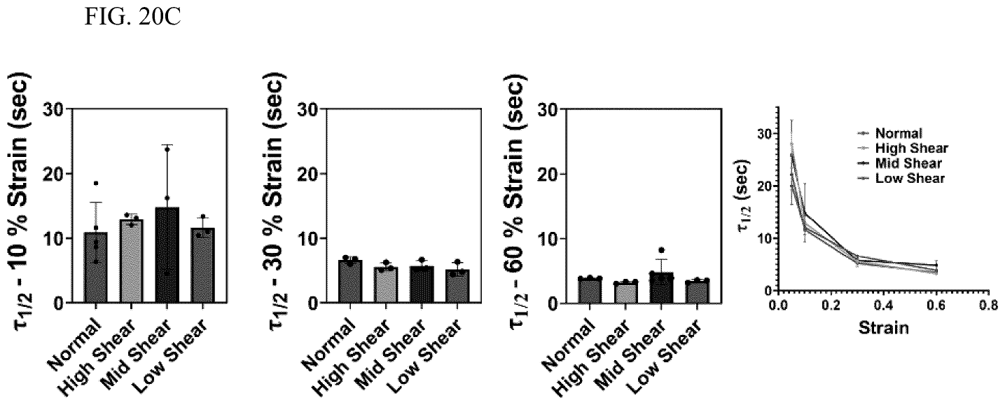

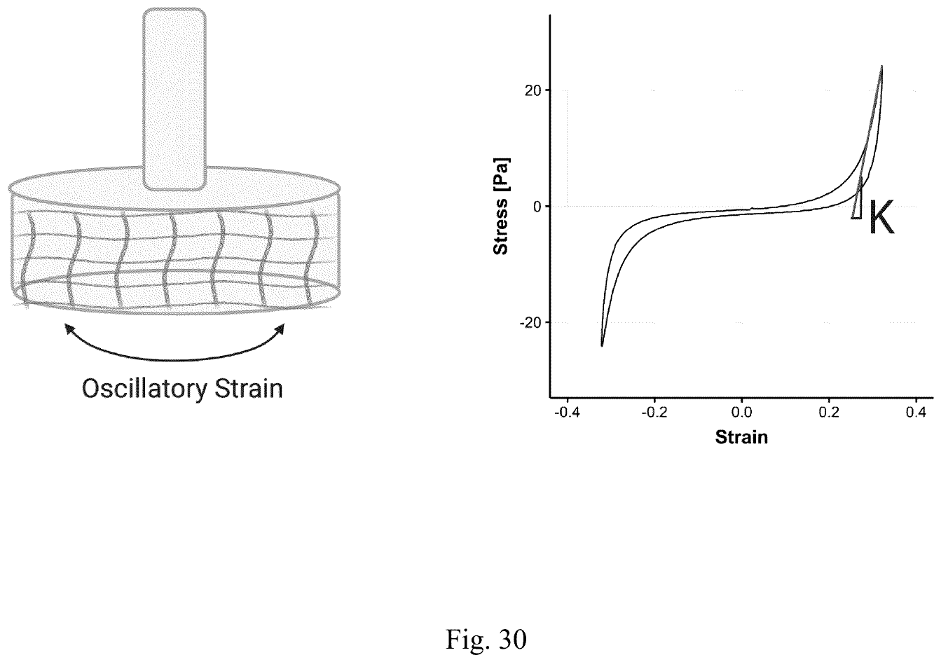

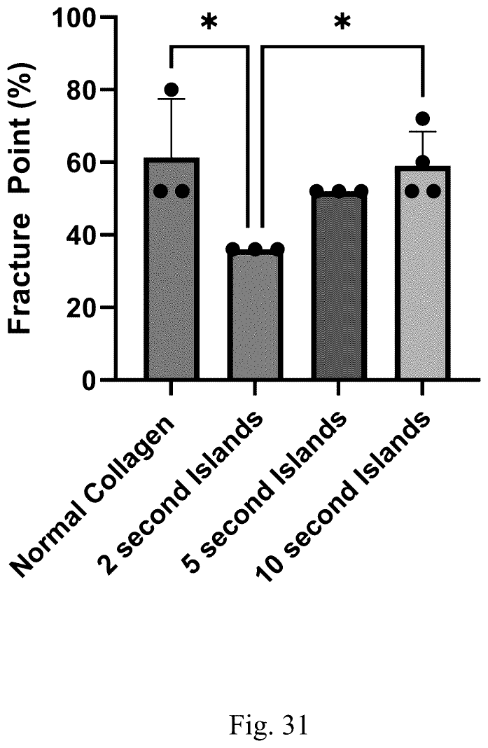

Given the distinct architectural organization of collagen islands, the bulk mechanical properties of these gels were interrogated. Rheological shear strain sweeps revealed that all collagen island architectures maintained their strain stiffening capabilities (FIG. 20A). Onset of strain stiffening occurred in all gels between 12-16% strain. A differential shear modulus K was defined to describe bulk stiffness as a function of strain (physical description in FIG. 30). High shear island gels were significantly softer than an isotropic collagen gel. In addition, high shear island gels reached a smaller plateau K and failed under lower strains (FIG. 20B, FIG. 31). This could be due to increased mechanical fragmentation introduced during gelation which resulted in less dense background fibers that strain- stiffen and fail under lower shear strain.

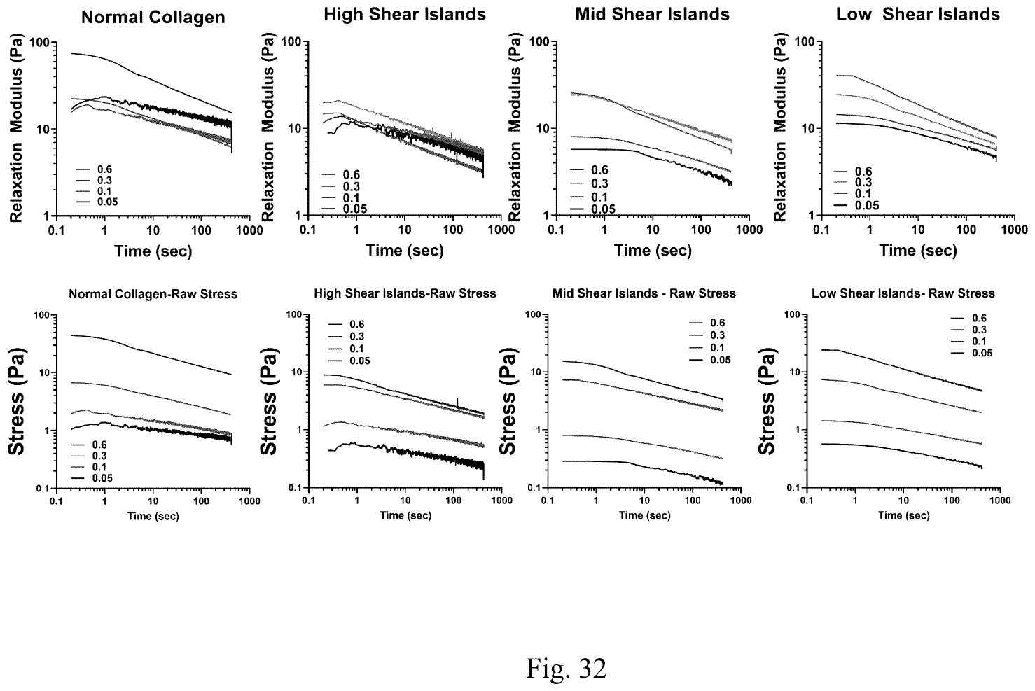

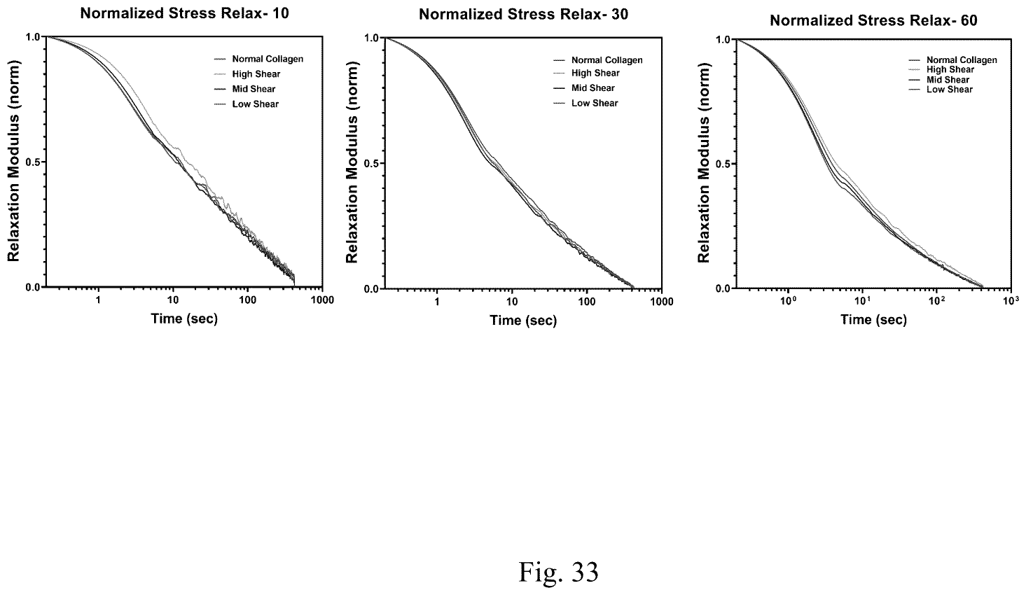

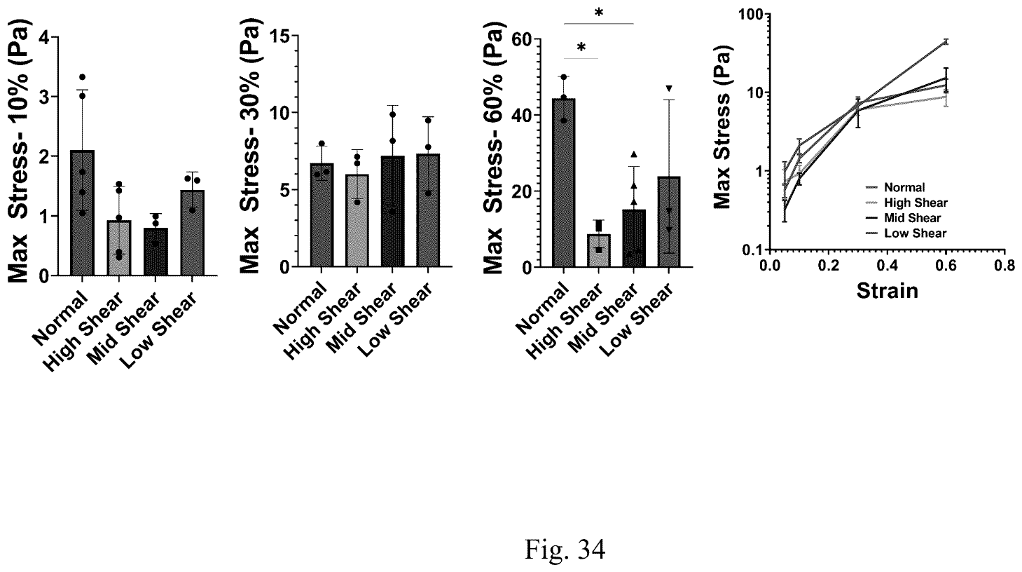

Stress relaxation tests were performed at different strains to determine the viscoelastic properties of the island gels. It was found that all gels exhibit strain dependent stress relaxation properties, in which higher strains lead to faster stress relaxation (FIG. 20B, FIG. 32). This behavior in isotropic collagen gels has been reported previously (Nam et al., PNAS 2016, 113, 5462-5497). No observable differences were found in normalized viscoelastic behavior between island gels at any strain (FIG. 20C, FIG. 33). Values were found to be around 10, 5, and 3 seconds for all gels at 10%, 30%, and 60% strain, respectively. It was found that max stress values increase with increasing strain, which was in accordance with strain sweep data (FIG. 34). This data suggested that the ability of collagen fibers to relax under applied strain was unaffected by mechanical fragmentation. Together, these results suggested that the island gels maintain both the ability to strain- stiffen and stress relax. Unlike normal collagen gels in which these properties must be tuned by changing either the density of collagen gels or by adding molecular crosslinkers (Bordeleau et al., PNAS, 2017, 114, 492-497), collagen island gels have tunable bulk mechanical properties without these alterations.

### Modulation of Cell Contractile Behavior by Heterogeneous Architecture

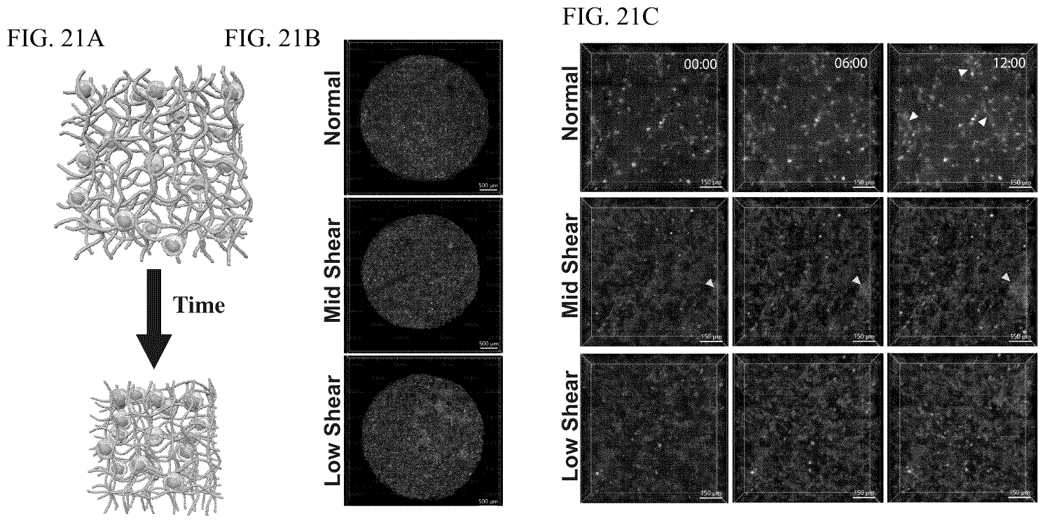

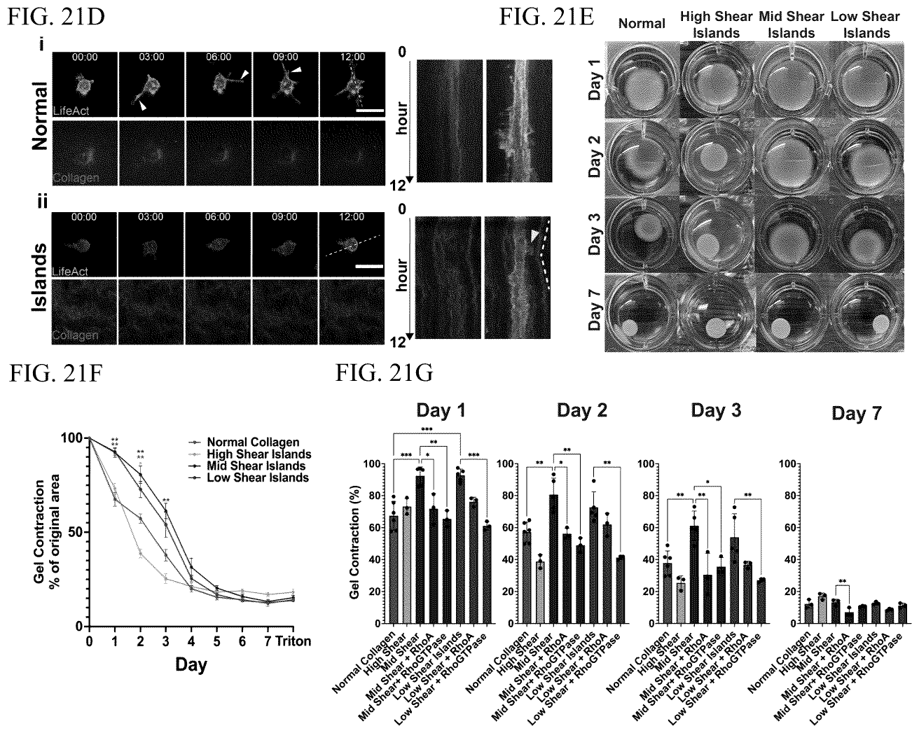

Gel compaction assays were performed in which D1 murine mesenchymal stem cells (MSCs) were embedded in gels and monitor gel area over time (FIG. 21A). Understanding MSC behavior in these gels are of particular importance for their use in tissue engineering. To initially understand cell behavior in these gels, gel compaction assays were performed in which 15 μl gels of different architectures are plated on microwell plates and imaged over 12 hours (FIGS. 21B and 21C). These plates were coated with 3% BSA to prevent collagen sticking to the plate. Over the course of the timelapse, densification and alignment profiles in the isotropic normal gels were observed (FIG. 21C, white arrows). Such profiles are indicative of local strain stiffening in the network (Han et al., Proceedings Of The National Academy Of Sciences Of The United States Of America, 2018, 115, 4075-4080). Conversely, cells were unable to deform collagen islands likely because they are too stiff to be mechanically remodeled. While all island gels showed very little plastic tracks, they do show densification of islands indicated by their compaction (FIG. 21C, yellow arrows). This suggested that cells were more able to remodel the normal (thin) collagen fiber network and the thin background fiber network which percolate between the islands than the islands themselves.

When cell-matrix interactions in the normal or mid shear island gels were further examined, differences in dynamic protrusion activity were noticed. It was found that MSCs were highly dynamic in normal gels and demonstrated the ability to form many long protrusions and densify collagen around themselves (FIG. 21D). However, MSCs cultured in island gels formed smaller protrusions and showed less densification of surrounding collagen. In addition, MSCs cultured in these gels could send out protrusions and cause movement of neighboring mesoscale islands (FIG. 21D, yellow arrow). It was also noted that once these protrusions retracted, the islands were able to recoil back near their original position, suggesting elastic deformations at this timescale (FIG. 21D, white dotted lines). Together, these data suggest that MSCs cultured in island gels have fewer dynamic protrusions and, therefore, are less able to initially non-elastically densify their local surroundings via nonelastic recruitment through protrusions (Malandrino et al., PLOS Computational Biology 2019, 15, e1006684). This may be due to fewer available thin fibers in the background of island gels for protrusions to be generated onto and to recruit. This may also be due to the background network being softer (due to reduced collagen concentration), thus reducing cell contractility via mechanosensing (Janmey et al., Physiological Reviews, 2020, 100, 695; Ghibaudo et al., Soft Matter, 2008, 4, 1836-1843).

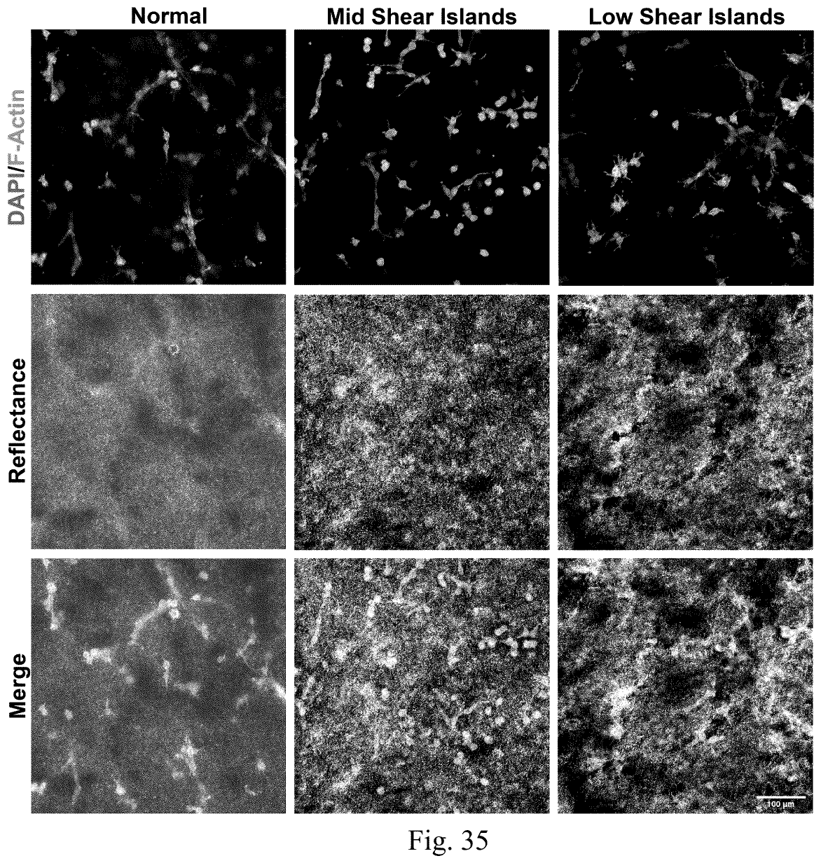

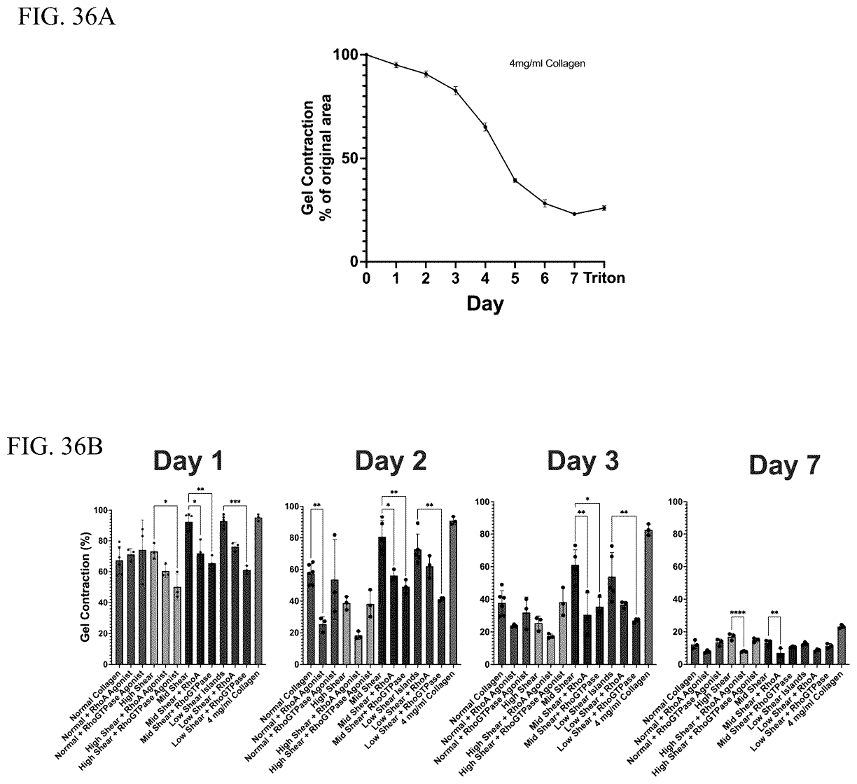

Standard gel compaction assays were performed and monitored compaction over the course of 7 days (FIG. 21E). Wells were again coated 3% BSA to prevent the collagen from sticking to the plate. It was found that the high shear island gels compacted significantly quicker than did the isotropic gel on days 2 and 3 but also eventually converged to the isotropic gel compaction area (FIG. 21F, FIG. 21G). This agreed with rheometry data which showed that high shear island gels are softer and can be strain stiffened at lower strains. Interestingly, it was found that mid shear and low shear island gels compacted significantly slower than isotropic 2 mg/ml gels over the course of the first 3 days but then eventually converged to the isotropic gel compaction area with the mid shear island gels compacting the slowest. Immunofluorescence of cells after 2 days of culture in compacting gels revealed no significant difference in cell morphology or cell proliferation (FIG. 35). To compare, gel compaction of a 4mg/m1 collagen gel was performed and it was observed that these gels compacted significantly slower than all island and normal gels and also compacted to a higher plateau area (FIG. 36A).

In addition to elastic deformation of fibers within the gel, cells dynamically remodel the matrix and plastically deform the gel (Malandrino et al., PLOS Computational Biology, 2019, 15, e1006684). Plastic deformation of fibers within gels can play a major role in gel mechanics which will in turn give rise to altered cellular response. To determine the amount of plastic deformation, all cells were removed or lysed with SDS after 7 days of culture. A 1-3% increase was observed in gel area, indicating that the majority of gel compaction was non-elastic (irreversible).

Given that MSCs cultured in the mid and low shear island gels compacted less quickly than those cultured in normal gels and that MSCs cultured in island gels showed fewer dynamic protrusions, gel compaction was sought to be rescued by increasing dynamic protrusion activity. Thus, MSCs cultured in mid or low shear gels were incubated with agonists for either RhoA or all RhoGTPases. These molecular targets are known to affect cytoskeletal dynamics and are key players in collagen remodeling (Nguyen et al., Communications Biology 2022, 5, 202; Carey et al., Integrative Biology 2016, 8, 821-835). It was found that treatment with RhoGTPase agonist was sufficient to increase gel compaction rate in mid and low shear gels to that of normal gels (FIG. 21G, FIG. 36B). Future dosing studies will further help dissect the individual role of different RhoGTPases and cytoskeletal factors in gel compaction in these environments. Together, these data suggest that these collagen island architectures can modulate compaction behavior of cells and that this can be further modulated by altering RhoGTPase activity.

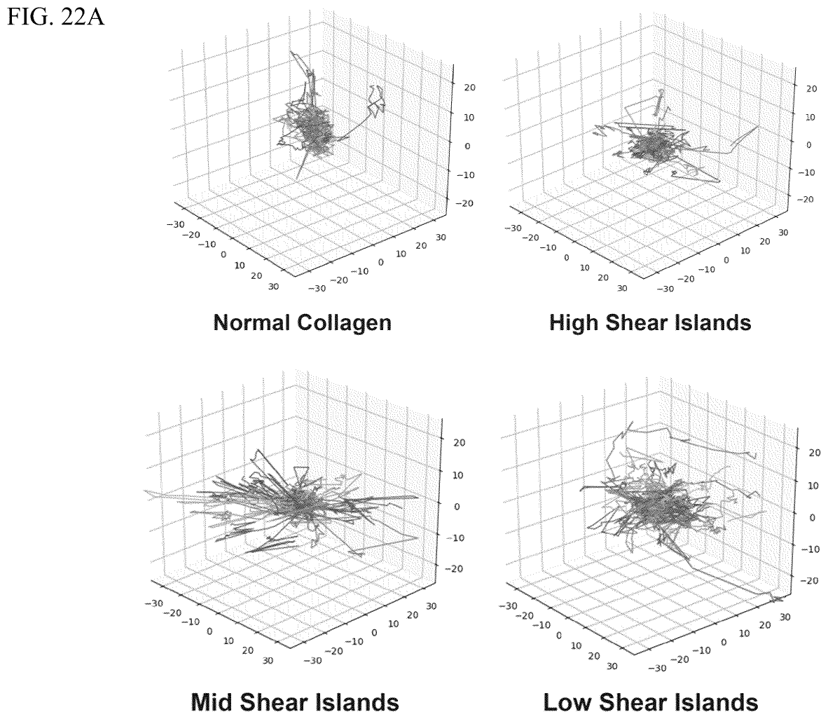

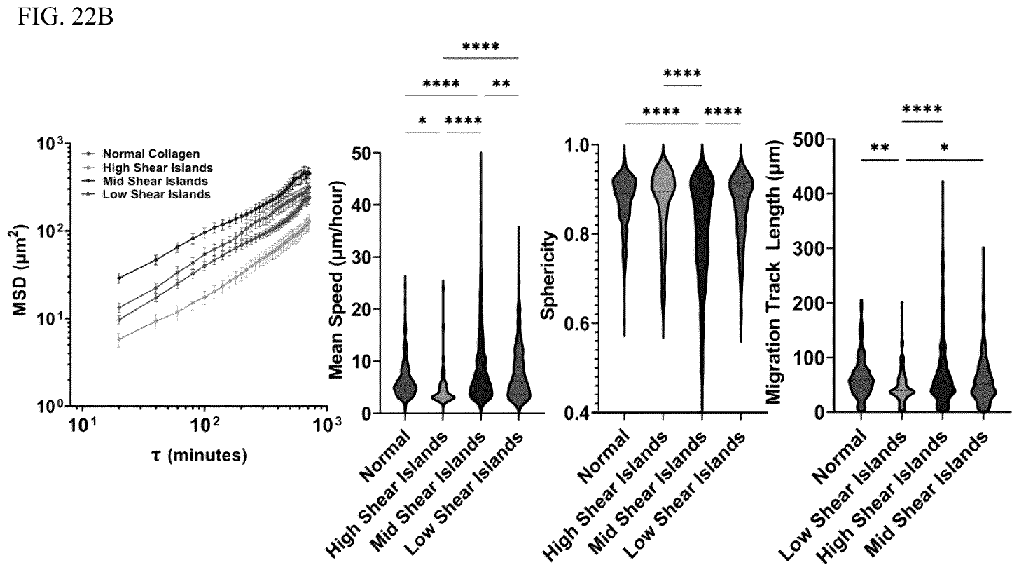

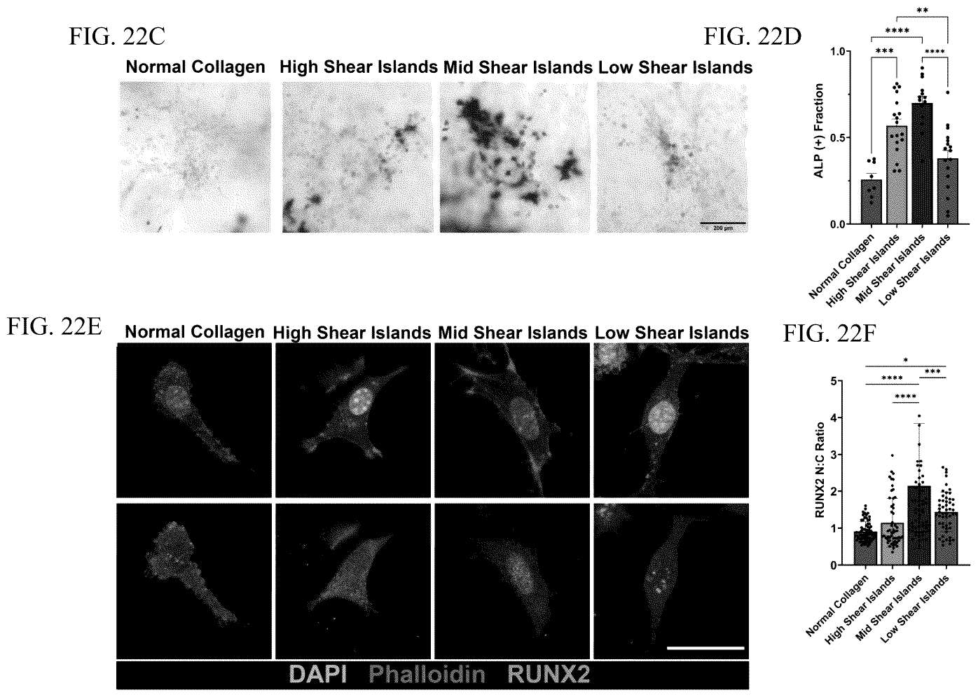

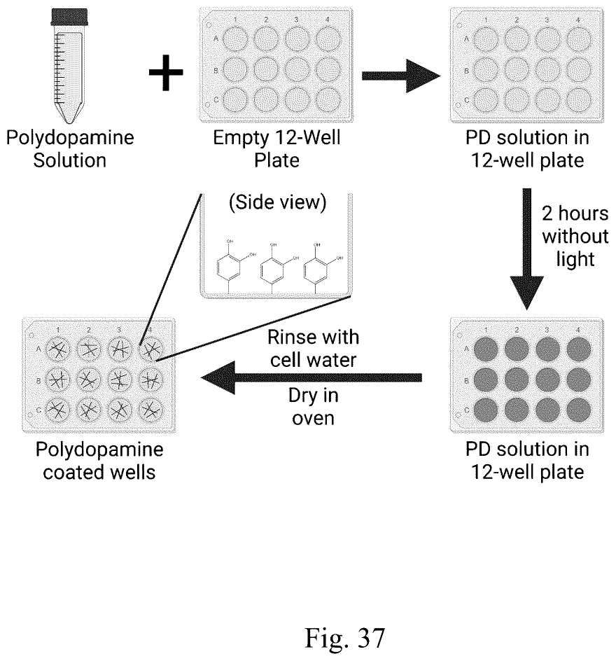

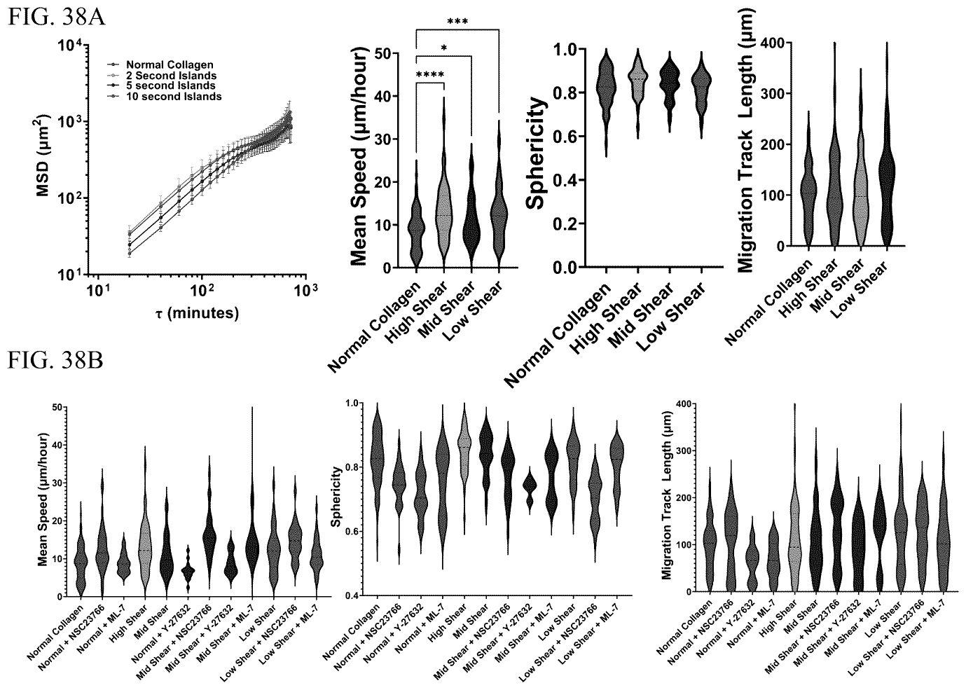

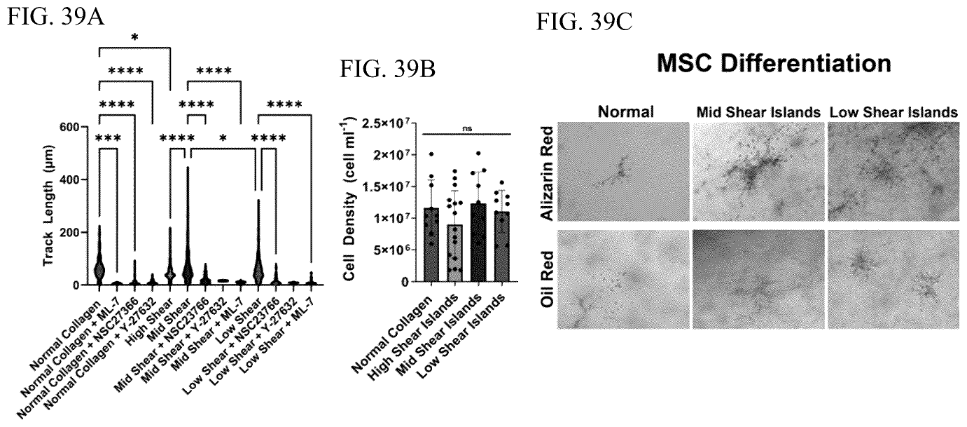

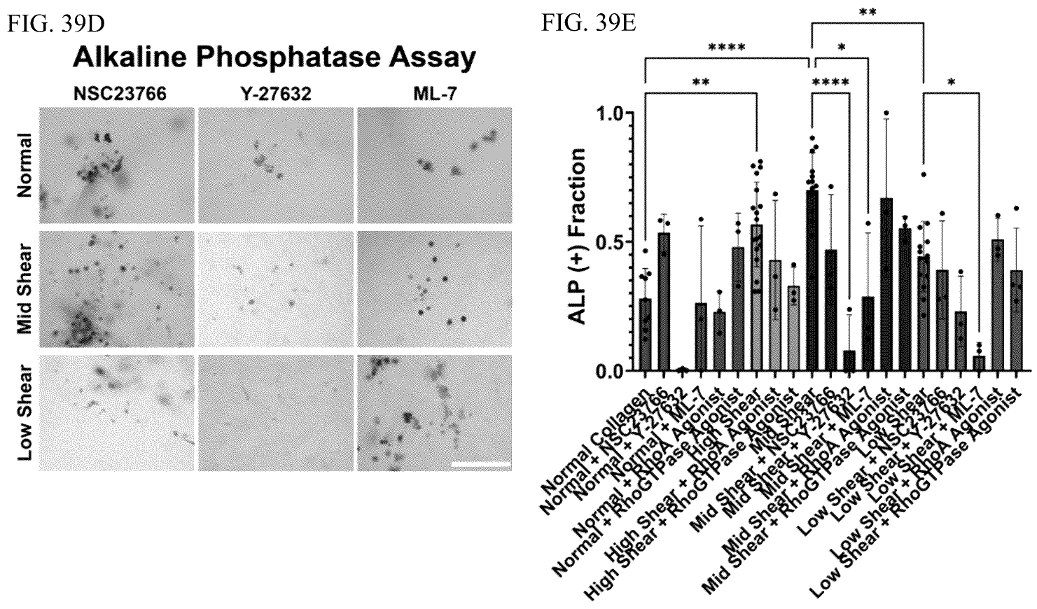

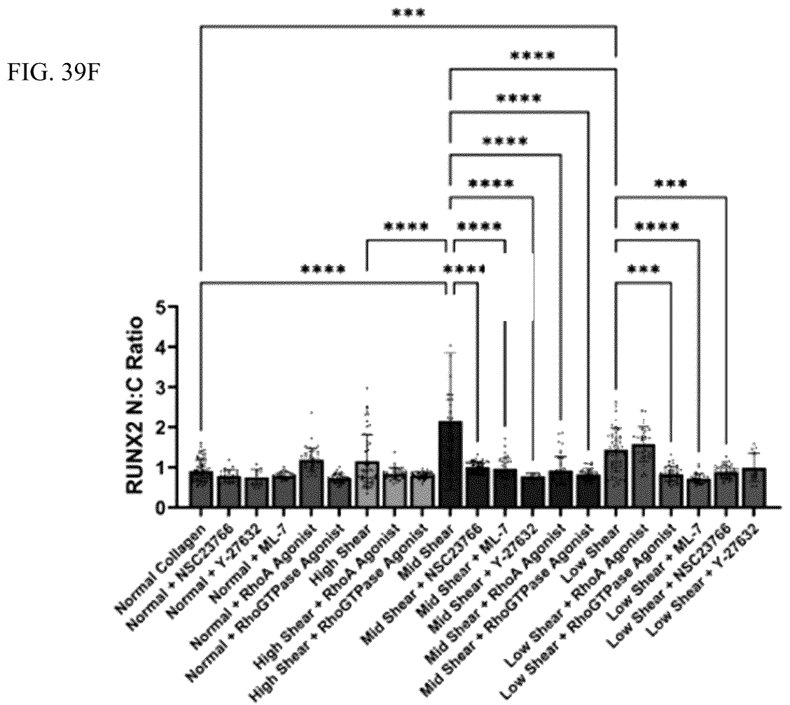

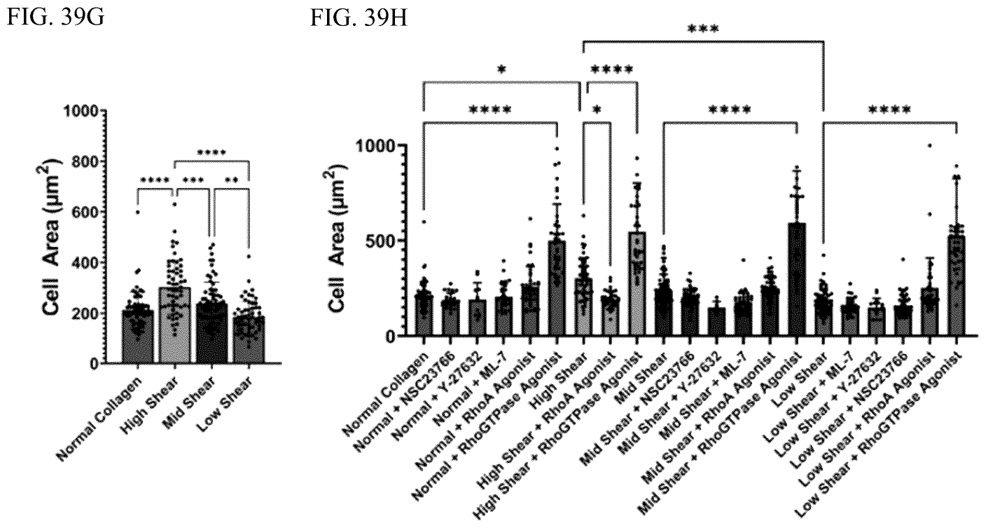

To determine how island architecture modulates stem cell behavior, DI MSC's were seeded in gels that were adherent to polydopamine covered glass (FIG. 37). Polydopamine allows for collagen to remain anchored to surfaces for imaging and prevents total compaction of the gel during experiments (Park et al., ACS Applied Materials & Interfaces 2019, 11, 23919-23925). MSC migration in gels were then visualized. To capture cell migration in these gels, a stable LifeAct-RFP dye was transduced into MSCs and then these encapsulated cells were imaged with time-lapse confocal fluorescence microscopy. Cell tracking analysis was performed to determine migration metrics (FIG. 22A). The cells both 1 day after seeding and 6 days after seeding were imaged for 12 hour timelapses. After 1 day of culture, higher mean speeds were observed in all island gels compared to normal gels but no differences in migration track length (FIGS. 38A and 38B). After 6 days of culture, differences in migratory behavior were observed between MSCs cultured in different island gels (FIG. 22B). Timelapses indicated that MSCs tended to migrate around the islands and moved through the background fibers and did not move through islands. MSC migration speed was slow in both the normal and high shear island gels, increases in mid shear island gels, and then decreased again in low shear island gels. This bell curve was observed in mean sphericity of cells and migration track length. As a control, cells were treated with inhibitors of myosin-based contractility (NSC23766 for Rac1, ML-7 for myosin light chain kinase, and Y-27632 for Rho- associated kinase (ROCK)) and decreases in cell migration track length were observed for all conditions (FIG. 39A). Together, these data suggested that migratory capability in these gels could be modulated by the ratio of background fibers to islands and that there is an optimal island architecture to promote migration.

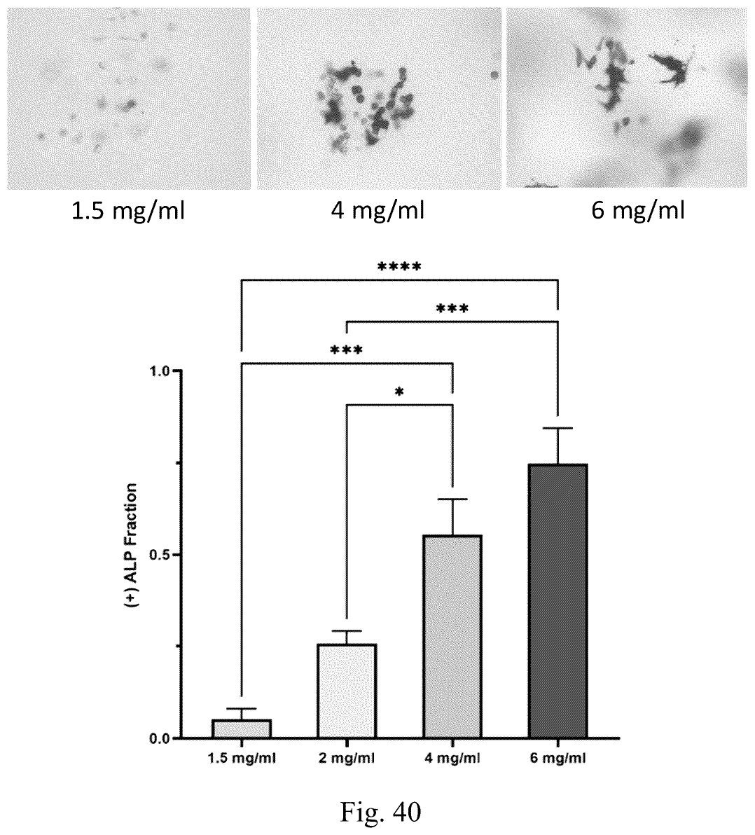

As a control to verify these results, MSCs were embedded on collagen gels of increasing density and, therefore, increasing stiffness. Indeed, we saw increased osteogenic commitment of cells in increasing collagen density (FIG. 40). Interestingly, osteogenic commitment of these MSC's also follows a bell-shaped curve (FIG. 22B). ALP signal was low in normal and high shear island gels, increased significantly in mid shear island gels, and then decreased in low shear island gels (FIGS. 22C and 22D). Differences in cell division are not detectable between island conditions (FIG. 39B). We further confirmed osteogenic commitment by staining for calcium deposition with Alizarin red (FIG. 39C). Since our cells were cultured in 50/50 osteogenic and adipogenic induction media, we also stained for adipogenic commitment but did not see significant adipogenic activity (FIG. 39C). Pharmacological inhibition of myosin-based contractility caused a decrease in ALP positive staining (FIGS. 39D and 39E).

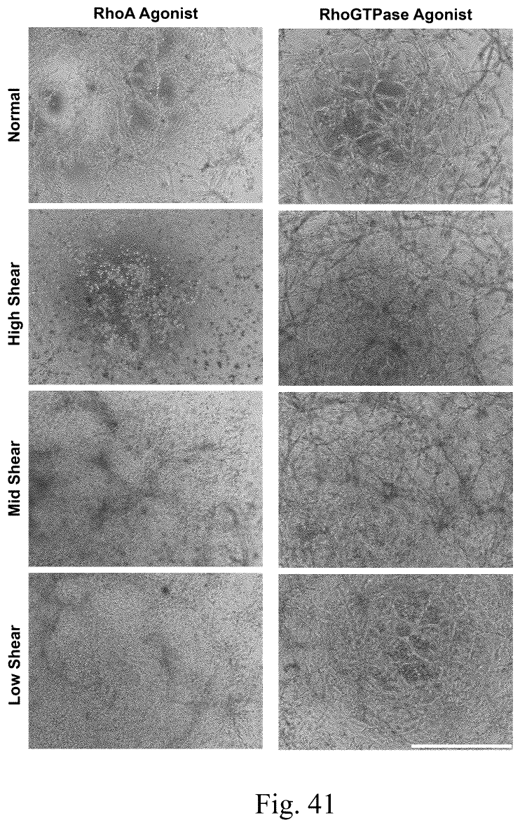

To further investigate this differentiation behavior, localization analysis was performed for RUNX2, a key transcription factor in osteogenesis (Zhao et al., Molecular Therapy: The Journal of The American Society Of Gene Therapy, 2005, 12, 247-253; Yang et al., Nature Materials 2014, 13, 645-652). A bell-shaped curve in RUNX2 nuclear localization was again observed with the highest amount of nuclear RUNX2 found in the mid shear gels (FIGS. 22E and 22F). Pharmacological inhibition of myosin-based contractility also inhibited RUNX2 nuclear localization (FIG. 39F). Interestingly, treatment of MSCs with either RhoA or RhoGTPase agonist actually decreased osteogenic commitment and promoted cellular network formation (FIG. 39G, FIG. 39H, FIG. 41). Cells are able to spread in all conditions, and there is heterogeneity in how much they spread (FIG. 39G). Their shapes can change dynamically over time. Even though cells can spread in all conditions, there is a bell-shaped curve in osteogenic commitment versus island architectures.

These results, which reveal a bell-shaped curve in osteogenic commitment in island architectures, could be explained by the complex architectures of these island gels. Though the bulk stiffness of these gels is soft, the islands provide high local stiffness which modulates stem cell behavior. This data suggests an optimal spacing of stiff inclusions to promote cell spreading. These results indicate an optimal island architecture for osteogenic differentiation which cannot be achieved in normal isotropic collagen gels.

### Differentiation of iPSCs in island architecture

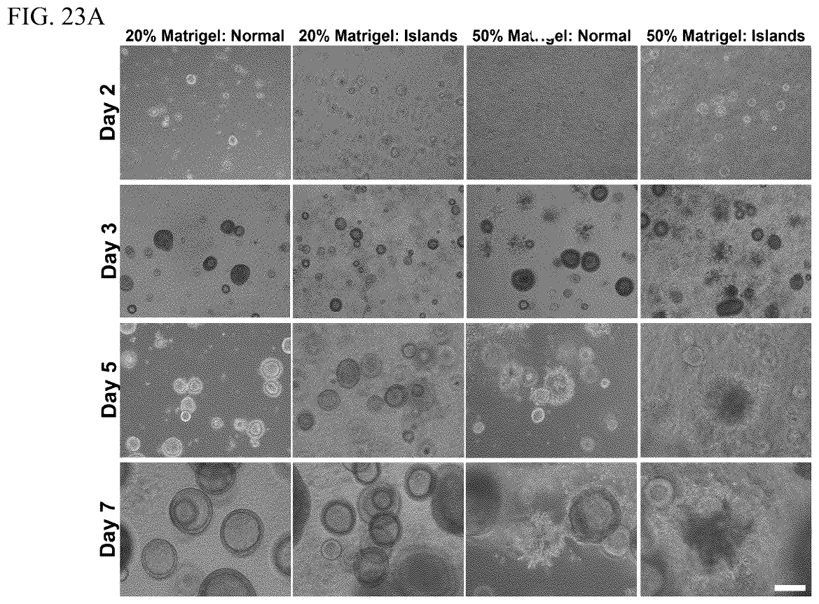

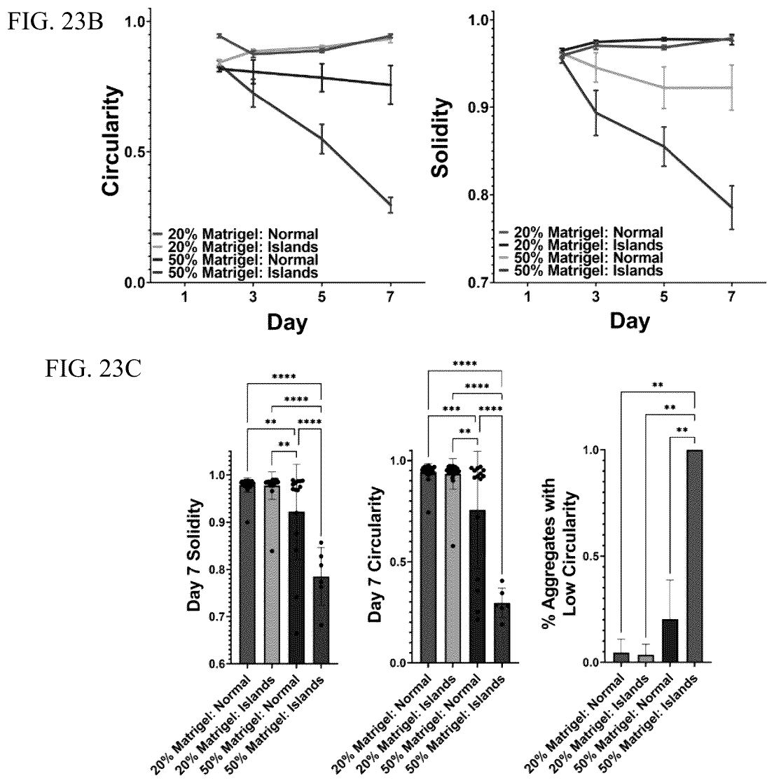

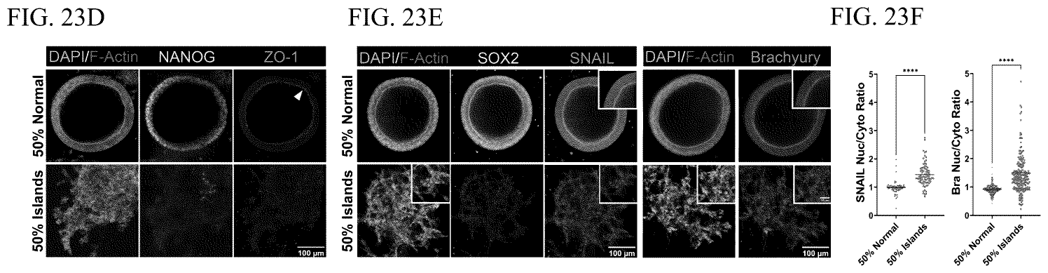

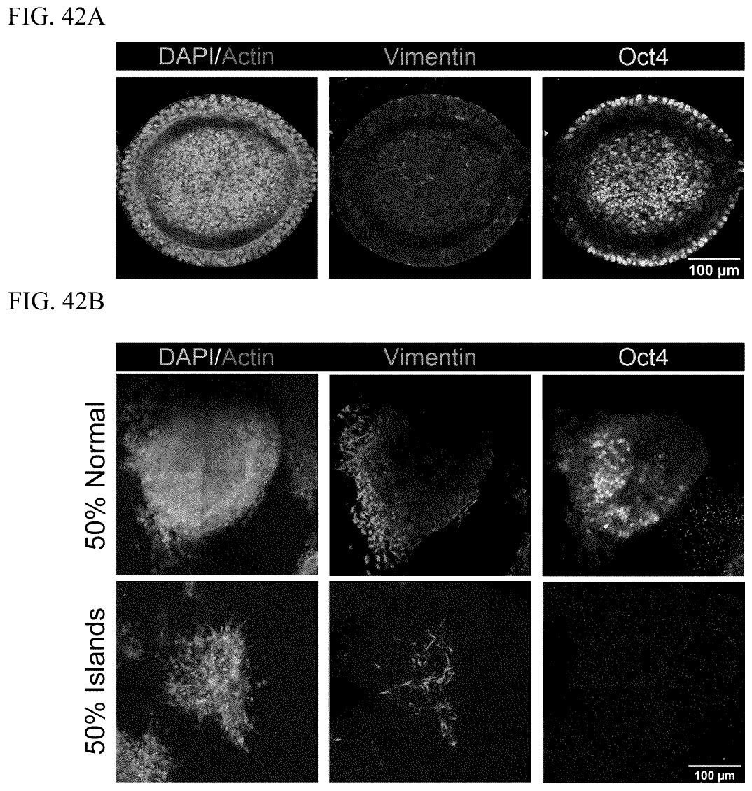

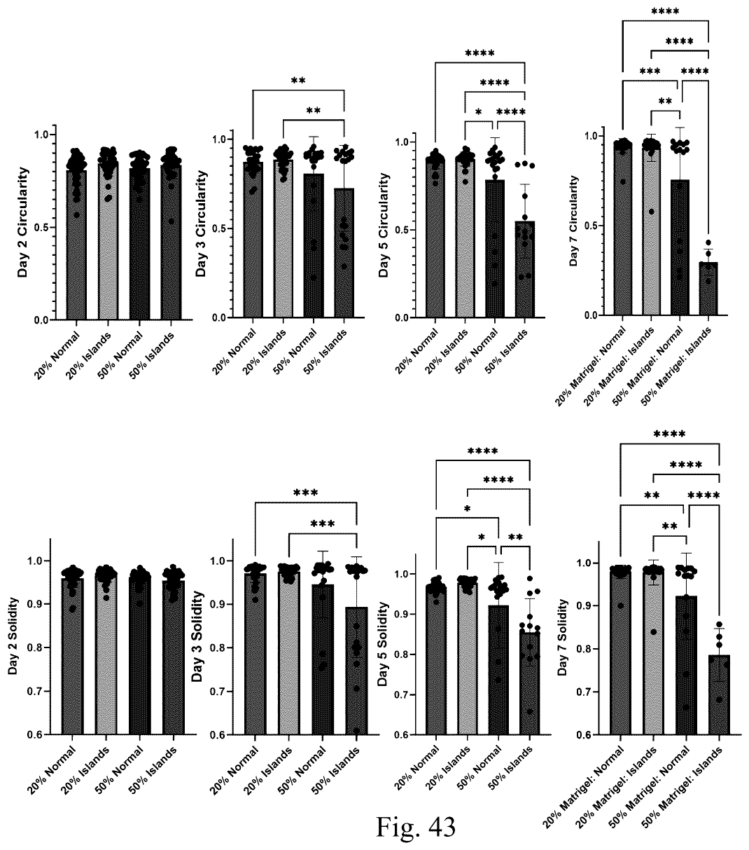

Given that these results showed that the mid shear island gels provided optimal cell behavior in terms of spreading and differentiation, it was next investigated how this architecture could affect iPSC behavior. Single cell iPSC suspensions were encapsulated in either normal or island gels and the amount of matrigel concentration was varied. iPSCs were cultured in conditions in which matrigel made up either 20% or 50% of the final volume, and PBS was added to the 20% matrigel solutions to create equal volume solutions. In both cases, normal gels or island gels made up 50% of the final volume. Growth of iPSCs were monitored over the course of 7 days without the addition of differentiation factors (FIG. 23A). The island architectures are fabricated before being added into matrigel. This eliminates the possibility of changes in collagen island gelation kinetics as the islands are already formed. As shown in previous studies (Simunovic et al., Nature Cell Biology 2019, 21(900-910); Stem Cell Reports 2015, 5(954-962)), iPSCs grown in pure Matrigel self-organized to form lumen-like structures and maintained their pluripotency after 7 days of culture (FIG. 42A). While cells grown in 20% matrigel solutions tended to form circular lumens, those grown in 50% matrigel solutions started showing invasive strands. Measurements of solidity and circularity showed that this phenotype appears after 3 days of culture and continued to diverge from the 20% conditions further over the course of the experiments (FIG. 23B, FIG. 43). Notably, roughly 30% of the iPSC lumens grown in 50% matrigel gels showed these invasive phenotypes whereas almost all the iPSC lumens grown in 50% island gels showed invasive strands, suggesting that the islands helped push the iPSCs to a differentiated state (FIG. 23C).

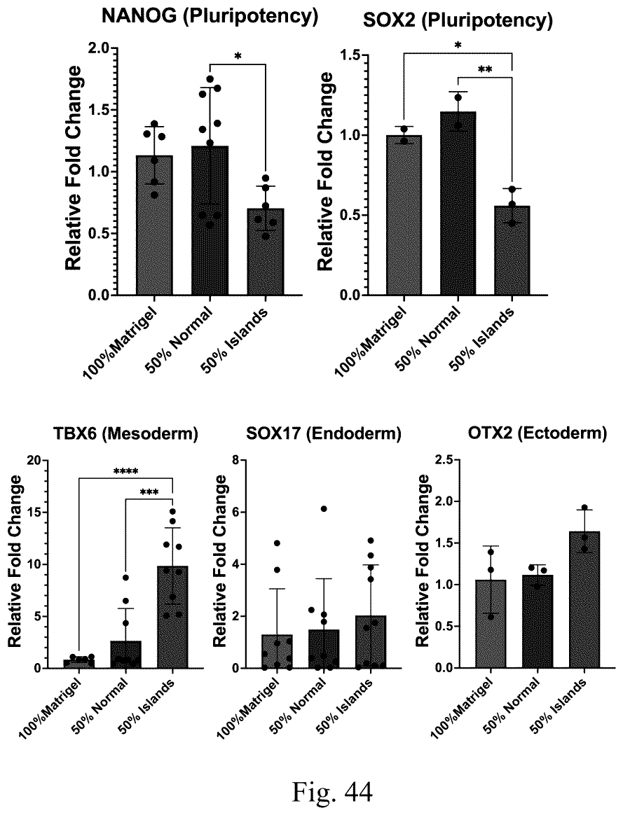

The effect island architecture could affect stem cell fate was next explored. Localization analysis was next performed on the canonical markers for pluripotency, ZO-1 and Nanog (Simunovic et al., Nature Cell Biology, 2019, 21, 900-910). Immunofluorescence staining of these structures revealed that cells cultured in 50% island gels lose both their stemness as well as their apical basal polarity, indicated by a loss of Nanog and ZO-1, respectively (FIG. 23D). Further confirmation of pluripotency loss was shown by a lack of SOX2 expression in 50% island gels. The cells were next stained for the canonical mesodermal markers Snail and Brachyury (Carver et al., Molecular and Cellular Biology, 2001, 21, 8184; Simunovic et al., Nature Cell Biology 2019, 21, 900-910). After 7 days of culture, cells cultured in 50% island gels show nuclear localization of both Snail and Brachyury, indicating mesodermal differentiation (FIGS. 23E and 23F). Mesodermal differentiation was further confirmed via qPCR and saw a significant decrease in expression of NANOG and SOX2 (pluripotency markers) and a sharp significant increase in TBX6 (mesodermal markers). No significant differences were observed in expression of either ectoderm (OTX2) or endoderm (SOX17) markers (FIG. 44). Expression of vimentin, a canonical EMT marker, was also found in invasive cells cultured in 50% normal gels and in all cells cultured in 50% island gels. The mechanism behind this differentiation pattern must be further explored. This phenomenon could be explained by several gel properties such as asymmetric distribution of ligands as well as heterogeneous local stiffnesses. Together, these data suggest that architectural cues, and notably islands, are sufficient to induce mesodermal differentiation of iPSCs. Overall, these island architectures may prove to be a useful tool to induce differentiation.

### Collagen Island Gels

Collagen island gels were developed which accurately depict features found in many real tissues. Island gels are relatively simple to manufacture as they only require mechanical shear to form and have tunable island size depending on mechanical shear frequency. Furthermore, these islands can be isolated from solution and resuspended in other natural ECM-derived matrices. Altogether, these island gels mimic tissue architecture, have tunable topographical and mechanical properties, and are compatible with modular assembly for tissue engineering.

The fluid dynamics model was able to obtain metrics of shear stress and energy introduced into the system. While this model does not consider key metrics such as gelation, it could give some predictive power in terms of defining the full phase space of collagen islands. The model allows for predictions on how changes in pipetting frequency, pipette geometry, and even distance of pipette from bottom of the tube could affect total energy output and, therefore, island architecture. Further studies will address how changes in these parameters will affect overall spacing and size of islands. Characterizing the phase space of these island gels will help guide optimal recipes for the assembly of mesoscopic ECM architectures and uncover key mechanical driving factors. This will allow for the study of physiologically mimicking heterogeneous and mesoscopic cues, which typical hydrogel gels do not offer but are important physiologically and pathologically (Provenzano et al., BMC Medicine, 2006, 4, 38; Cicchi et al., Journal of Biophotonics, 2010, 3, 34-43).

Bulk mechanical properties of these island gels are tunable. Their bulk stiffness can be modulated. However, AFM is still required to probe the local mechanical properties of these island architectures. In addition, while viscoelasticity has been shown to be a determinant of cell behavior in 3D hydrogels, bulk normalized viscoelasticity does not seem to be affected by introduction of island architecture. However, this constant viscoelastic behavior remains poorly understood. It is hypothesized that, while the network of the collagen has experienced significant rearrangement, longer fibers (which are the main contributors of viscoelasticity) remain unaffected. A particularly useful tool for teasing apart these mechanics are discrete fiber network models, which have been used extensively to understand local network behavior of various biomaterials under applied stresses (Mak et al., Nature Communications 2016, 7, 10323; Computational and Structural Biotechnology Journal, 2020, 18, 3969-3976). Future studies will incorporate these models to better understand the mechanical contributions of these islands to bulk properties. Overall, these data suggests that local stiffness, island spacing, and connections between islands are all important cues that affect cell behaviors in these gels.

### Cell Culture

MDA-MB-231s (Lifeact GFP) were a gift from the Lauffenburger lab. They were cultured in DMEM (Gibco™11965092) with 10% FBS (Gibco™16000-044) supplemented with 1% penicillin-streptomycin (5,000U/ml, Gibco™15070-063). Normal human lung fibroblasts (ATCC, PCS-201-013) were cultured in Lonza FGM-2 BulletKit (CC-3132) or RPMI1640(Gibco™11875-093) with 10% FBS and 1% Pen-Strep. Cells were all maintained at 37.0 and 5% CO2.

D1 MSC cells were obtained from the American Type Culture Collection (ATCC; Manassas, VA, USA). ATCC validated all cell lines by Short Tandem Repeat Analysis. Cells were maintained at 37° C., 5% CO2. D1 MSCs were maintained in Dulbecco's Modified Eagle's Medium (DMEM) medium supplemented with 10% fetal bovine serum, 1% L-glutamine, and 1% penicillin/streptomycin. Media was changed every other day, and cells were split every 3-4 days. To generate MSC cells expressing LifeAct-GFP, lentiviral particles containing a pLentiCMV-MCS-LifeAct-GFP vector were used. Cells were selected with puromycin (10 μg ml⁻¹) and were cultured at no more than 80% confluency at no greater than passage 25 in serum-supplemented DMEM.

The hiPSCs were a gift from Dr. Stuart Campbell (Department of Biomedical Engineering, Yale University). hiPSCs were cultured on TC-treated 6-well plates (Costar, Corning) coated with lactose dehydrogenase elevating virus (LDEV)-free human-embryonic-stem-cell-qualified Matrigel (Corning, 354277) in mTeSR1 media (STEMCELL Technologies) at 37° C. in 5% CO2. Media was changed daily. hiPSCs cultured in mTeSR1 were passaged at 70% confluency as pluripotent aggregates with manual selection using ReLeSR (STEMCELL Technologies) following the manufacturer's protocol.

### Collagen Gel

Each collagen gel was made by adding sufficient 0.5 N NaOH to neutralize a mixture of double-distilled H2O, 10×PBS, and acetic-acid-solubilized type I rat tail collagen (Corning, Corning, NY, USA) on ice for a final collagen concentration of 2 mg/ml. Island gels were made by first neutralizing 350u1 of collagen. Stained collagen gels were made by incorporating 10% 647-ester dye (ThermoFisher) stained collagen. The gels were then allowed to sit at room temperature for 6.5 minutes. Finally, the gels were sheared with a p200 pipette tip set to 175u1 at a specified frequency for 3 minutes before being plated. Prior to depositing the collagen, plates used for collagen gel seeding were surface-coated with polydopamine, as previously described (Park et al., ACS Applied Material Interfaces 2019, 11, 23919-23925; Lee et al., Science 2007, 318, 426-430; Nguyen et al., Communications Biology, 2002, 5), which allowed collagen gels to stick to the surface of the plates to prevent detachment of the gel. After plating, gels were transferred to an incubator at 37° C. with 5% CO2 for 1 hour.

### Encapsulation of iPSCs within Hydrogels

hiPSCs at 70% confluency or less were passaged as single cells using Accutase (STEMCELL Technologies), washed in mTeSR1 media, and resuspended as a single-cell suspension in mTeSR1 media with 10×10-6 m ROCK inhibitor (Y-27632, STEMCELL Technologies) to prevent dissociation-induced apoptosis. For 50% collagen gels, isotropic or island gels were made and mixed in equal parts on ice with Matrigel (Corning, 354277). For 20% matrigel:collagen gels, isotropic or island gels were made and mixed with Matrigel and PBS on ice such that the resulting gel was 20% Matrigel by volume. Collagen gels were made, and an appropriate number of cells were mixed to achieve a final concentration of 100000 cells/ml. After gelation, mTeSR1 media with 10×10−6 m ROCK inhibitor was added. 24 h post encapsulation, the media was changed to mTeSR1 which was replenished daily.

Encapsulation of MSCs within hydrogels

D1 MSCs or D1 MSC-LifeActRFP cells were mixed with the collagen solution prior to gelation to achieve a final cell concentration of 15000 cells/ml. Cells were cultured in 50%/50% mixture of the osteogenic induction medium and adipogenic induction medium (differentiation medium). The differentiation medium contained 50 μg/ml L-ascorbic acid (MilliporeSigma), 10 mM β-glycerophosphate (MilliporeSigma), and 0.1 μM dexamethasone (MilliporeSigma) in the DMEM growth medium.

MSC time lapses were performed in 12 well glass bottom dishes which had been polydopamine coated. 12 hour time lapses were taken on Day 1 and Day 6. Automatic cell segmentation was obtained using Imaris (Oxford Instruments). Manual filtering was done to determine which cells were to be used for downstream analysis. From these segmentations, cell movement was tracked and metrics such as mean speed, sphericity, and migration track length over the timelapse were obtained. Mean speed is defined as the average of instantaneous speeds between timepoints.

### MSD Image Analysis

Custom Python3 scripts were used to calculate MSD plots. Trajectories were exported to Python to calculate average speed and mean squared displacement. Average speed specifically refers to the mean of the absolute value of the net displacement of the cell center per hour. Mean squared displacements MSD(n) were computed with the following equation:

$$M S D ( n ) = \frac{1}{( N - n + 1 )} {\sum }_{i = 0}^{N - n} [ {( {x}_{i + n} - {x}_{i} )}^{2} + {( {y}_{i + n} - {y}_{i} )}^{2} + {( {z}_{i + n} - {z}_{i} )}^{2} ]$$

where N is the total number of steps, n is the n-th step, and x,y,z are the x,y,z coordinates, respectively.

Gel Compaction

Gel compaction experiments were performed in 24 well plastic bottom plates. Wells were incubated with 3% BSA for 1 hour and then washed with PBS to avoid adhesion of the gel to the plate. D1 MSCs were mixed with 500 μL of collagen solution prior to gelation to achieve a final cell concentration of 600000 cells/ml. Cells were cultured in the differentiation medium described above, and media was changed every day. Images of the gels were taken every day for 7 days. After 7 days of culture, gels were treated with an 8% Triton and 0.125% Trypsin-EDTA solution for 30 minutes to decellularize gels and observe the degree of plastic deformation. Images of the gels were taken after decellularization. Timelapses of gel compaction assays were performed in 15 well plates (Ibidi). Cells were incubated with 3% BSA for 1 hour and then washed with PBS to avoid adhesion of the gel to the plate. D1 MSCs were mixed with 15 μL of collagen solution prior to gelation to achieve a final cell concentration of 600000 cells/ml. Cells were cultured in 50 μL of the differentiation medium described above. Images were taken every 6 minutes for 12 hours.

### MCF10A maintenance and acini formation

MCF10As (ATCC, CRL-10317™) were cultured with MCF10A growth medium which includes DMEM/F12 (Gibco™, 11330-032) supplemented with 5% horse serum (Gibco™16050122), 20 ng/ml EGF, 0.5 mg/ml hydrocortisone (Sigma H- 0888), 100 ng/ml cholera toxin (Sigma C-8052), 10 ug/ml insulin (Sigma 1-1882) and 1% penicillin-streptomycin (5,000U/ml, Gibco™15070-063). To produce MCF10A acini, 24 well plates were first coated with a thin layer of pre-thawed Matrigel (Corning 354230) and then kept in incubator for 30 minutes to allow Matrigel gelation. Around 5K to 10K MCF10A single cells were then seeded on top of the Matrigel and maintained with MCF10A assay medium in 37.C, 5% CO2 incubator until day4. MCF10A assay medium has the same components with MCF10A growth medium but with 2% horse serum and no EGF. MCF10A maintenance and acini formation protocols are adopted from the Brugge lab. Cell recovery solution (Corning354253) was used to extract MCF10A acini from Matrigel following manufacturer's protocol. Briefly, MCF10A acini (in Matrigel) were first washed with ice cold PBS 3 times followed by 1 hr ice incubation in cell recovery solution. Then MCF10A acini were spun down and washed in ice cold PBS for 3 times at 70 g for 2 minutes in pre-chilled centrifuge. Washed MCF10A acini were seeded in normal or thick collagen for further study.

### 3D Tissue Culture in Normal Collagen

Acid solubilized collagen I (Corning354249) was first neutralized with NaOH and then diluted with suspended cells (or acini) to a final concentration of 2 mg/ml or 1 mg/ml (MCF10A study) on ice. Culture media were added after 1 hr gelation at 37° C. For labeled ECM studies, the initial collagen solution was labeled with Alexa Fluor 647 NHS Ester (Succinimidyl Ester) and dialyzed as before (Debnath et al., Methods, 2003, 30, 256-268). To note, the surface of the multi-well plates used for imaging were all pre-coated with polydopamine to anchor the collagen gel (Lee et al., Science, 2007, 318, 426-430; Yu et al., RSC Advances, 2014, 4, 7185), including thick collagen gels.

### 3D Tissue Culture in Thick Collagen

Acid solubilized collagen I (Corning354249) was neutralized with NaOH in the same way as in normal collagen preparation mentioned above (step 1 in FIG. 1D). Then the solution was kept still in room temperature for around 6.5 to 7.5 minutes (step 2) to allow initial nucleation. In step 3, wide-bore tips were used to gently pipette up and down the collagen while warming up the gel with fingertips. Mixing is stopped when the collagen solution becomes cloudy. The turbidity of the cloudy thick collagen after complete gelation (without adding medium or PBS) at A405 should fall around 0.4 to 0.5. The cloudy collagen solution can be directly mixed with cells (acini) and aliquoted into polydopamine coated wells followed by 1 hr gelation at 37° C. Alternatively, the cloudy collagen solution can be kept on ice for up to 30 minutes before mixing with cells. In this way, the cell mixing step needs to be prolonged accordingly before aliquoting (i.e., the longer the ice incubation, the longer the cell mixing is needed to retain collagen clusters). To note, over mixing will result in collapsed (non-cohesive) scaffold. The other steps are the same with standard 3D tissue culture. In all cell seeding experiment, 2 mg/ml thick collagen was used unless otherwise noted.

### Automatic Collagen Mixer

To further validate the replicability of the unique Collagen I architecture, a computer-controlled extruder was designed and constructed. Conventional off the shelf syringe pumps offer the accuracy required for this application, however, they tend to be heavy, bulky, costly and few offer the option of running custom pumping sequences without bypassing the whole controller board and connecting directly to the motor. It was therefore decided that to achieve the desired pumping in/out cycle, the best option would be to design a linear actuator that could operate any syringe and would be light enough to attach to a scaffold over the hot plate. The main components of the actuator are a 3D-printed frame, a Nema 17 stepper motor, a A4988 stepper driver, a set of 20T/60T GT2 gears, a T8 2 mm pitch lead screw, and an Arduino Uno microcontroller. The complete structure was printed in about 11 h using an Elegoo Saturn resin printer. The Arduino IDE was used to program and upload the code on the board. Using the known gear ratios and rotational-to-linear motion conversion of the lead screw, the speed of the stepper motor was adjusted to obtain the desired flow rates. The complete collagen shearing system can be built for less than $200.

### Migration Inhibition Drug Assay

Drugs were reconstituted and stored following manufacturers' recommendations and diluted in culture media to working concentrations. Specifically, 20 μM pan-MMPs inhibitor GM6001(Abcam, ab120845), 30 μM ROCK inhibitor Y27632 (Abcam, ab144494), 10 μM Cdc42 inhibitor ML141 (Calbiochem®217708), 50 μM formin inhibitor SMIFH2 (Abcam, ab218296), 50 μM ARP2/3 inhibitor CK666 (Abcam, ab141231), 100 μM Rac1 inhibitor NSC23766, 20 μM Rac1 inhibitor EHT1864 (Cayman 17258) were used. Fresh media mixed with specific drug were prepared right before experiment and replaced daily. In the drug assays, at least two independent experiments were performed with at least two replicates (technical duplicates) in each experiment.

### Turbidimetry

The microplate reader was first pre-heated to 37° C. For normal collagen measurement, acid solubilized collagen was neutralized and diluted to 2 mg/ml as above-mentioned. Ice-cold collagen solution was aliquoted into 96 well plates (80 μl per well) and absorbance at 405 nm (A405) was tracked every minute for 20 minutes. For thick collagen measurement, after following the standard thick collagen protocol to step 4 (without ice incubation), thick collagen solution was aliquoted into 96 well plates (80 μl per well) and readouts of A405 were recorded every minute for 20 minutes.

### Immunostaining

Culture media were first removed and then the gels were rinsed 3X gently with 1×PBS. Cells were then fixed with 4% formaldehyde for 30 min and permeabilized by 0.3% Triton X-100 for another 30 min. After fixation and permeabilization, cells were blocked with 1% BSA followed by primary antibody incubation (1:200), 3×PBS wash and secondary antibody incubation (1:500) supplemented by Hoechst stain (1:2000) and Alexa Fluor™647 Phalloidin (Invitrogen™A22287, 1:200). Fluorescence imaging were acquired within one week after immunostaining.

### Immunofluorescence Staining

Gels were rinsed twice with PBS and fixed with 4% paraformaldehyde (Thermo Fisher Scientific) in PBS for 25 min at room temperature. Following fixation, gels were permeabilized with 0.2% Triton X-100 in PBS for 1 h at room temperature. They were then blocked with 3% BSA in PBS containing 0.01% Triton X-100 for 3 h at room temperature. The samples were then incubated for 24 hat 4° C. with the primary antibody Nanog (1:100; α-Rabbit; Cell Signaling Technologies), SOX2 (1:100; α-Rabbit; Cell Signaling Technologies), Oct4 (1:200; a-Rabbit; Cell Signaling Technologies), Vimentin (1:200; α-mouse; Santa Cruz), SNAIL (1:200; α-Goat; R&D Systems), Brachyury (1:100; a-Goat; R&D Systems), or RUNX2 (1:100; Mouse; Cell Signaling Technologies). After washing for 3-5 h at room temperature, samples were incubated overnight at 4° C. with secondary antibody Alexa 647 goat-a-rabbit (1:1000 in PBS; Invitrogen), Alexa Fluor donkey-α-goat (1:1000 in PBS; Invitrogen), or Alexa 488 goat-α-mouse (1:1000 in PBS; Invitrogen), DAPI (1:2,000, Invitrogen) and phalloidin-Alexa 555 (1:500; Abcam). After at least 3 hours of washing, samples were mounted in Vectashield (Vector Laboratories) before imaging.

### ALP Staining

ALP was stained with a FastBlue working solution of 500 μg/ml Fast Blue BB (MilliporeSigma) and 500 μg/ml naphthol-AS-MX (MilliporeSigma) phosphate in an alkaline buffer (100 mM Tris-HCl, 100 mM NaCl, 0.1% Tween-20, 50 mM MgCl2, pH=8.2). Fixed samples were first washed three times in DPBS, equilibrated in alkaline buffer for 15 min, then incubated in FastBlue working solution for 60 min at room temperature. The samples were then washed in alkaline buffer for 15 min followed by 15 min in DPBS. For Oil Red staining, gels were equilibrated with 60% isopropanol for 30 minutes before being incubated in Oil Red in 60% isopropanol for 1 hour. Gels were then washed three times with water for 1 hour before imaging. For Alizarin Red staining, fixed samples were first equilibrated with water for 30 minutes. Gels were then incubated in 2% ARS solution (Sigma) for 30 minutes. Gels were then washed three times with H2O for an hour. Samples were then imaged after 7 days on a Leica DMH transmitted light microscope.

### mRNA Quantitative Testing

To quantify stem cell differentiation, mRNA from iPSCs cultured in gels for 7 days were extracted. At least 3 gels per replicate were suspended in Trizol and homogenized with a 20G needle. RNA was isolated by phenol-chloroform extraction and subsequent RNA extraction columns from the RNEasy Extraction Kit (Qiagen). 5 μg of RNA were reverse transcribed and amplified using the iTaq Universal SYBR Green One Step Kit (BioRad). Samples were analyzed using the Bio-Rad iTaq Universal Probes One-Step Kit in 20-μl reactions run at 50° C. for 10 min and 95° C. for 1 min, followed by 40 cycles of 95° C. for 10 s and 60° C. for 2 minutes per the manufacturer's recommendations. Reactions were performed on an Applied Biosystems 7500 instrument.

### Imaging

Imaging was performed using a Leica SP8 confocal microscope. Time-lapse imaging (XYZT mode) was taken with a 20× objective (0.75 NA), with time interval set to 3 minutes or 6 minutes and z-step size set to 2 μm. Day0 time-lapse imaging was acquired between around 0hr to 12 hrs post embedding cells into gels. Day1 time-lapse imaging was acquired between around 24 hrs to 36 hrs post gel-embedment. Overview tilescan images were taken at day1, day2 or day5 respectively.

### Confocal Microscopy

A Leica SP8 laser scanning confocal microscope with a x10 objective or x 5 objective (Wetzlar, Germany) was used for live imaging of MSC's. The 10x objective was used for 7 day time lapse experiments, and Z-stacks were taken with a thickness of 2 um. 12 hour time lapses were taken on Day 1 and Day 6. The 10x was used for 12 hour gel compaction experiments, and Z-stacks were taken with a thickness of Sum. A temperature of 37° C. and a 5% CO2 atmosphere were maintained using a humidified OKO labs live-cell imaging incubator. 3D fluorescence images were taken with either a 20x objective or 40x water objective. 3D Z stacks were taken with a thickness of 2 um. Cell and nuclear outlines were manually traced in ImageJ.

### Image Analysis

To track cell migration, XYZT data were first reduced in dimensions by conducting standard deviation projection along the z-axis. The XYT data were then processed by TrackMate in Fiji and produced cell trajectories. Trajectories were exported to MATLAB to calculate average speed and mean squared displacement. Average speed specifically refers to the mean of the absolute value of the net displacement of the cell center per hour. Mean squared displacements were computed with the following equation (Gorelik, Nature Protocols 2014, 9, 1931-1943):

$$M S D ( n ) = \frac{1}{( N - n + 1 )} {\sum }_{i = 0}^{N - n} [ {( {x}_{i + n} - {x}_{i} )}^{2} + {( {y}_{i + n} - {y}_{i} )}^{2} ]$$

In this equation, N is the total step number, n is the n-th step, x,y are the x,y coordinates respectively. For morphology analysis, z projected tilescan images were segmented and analyzed by in-house MATLAB codes. Segmentation performance was validated, with wrong segmentation manually corrected or removed. For difficult automatic segmentation data such as the Y27632 treated condition, manually tracked data were supplemented. The definitions of elongation, compactness, sphericity and shape variance were described in FIG. 4B (Liu, Scientific Reports 2022, 12, 791). A custom ImageJ script was used to derive the colocalization of the YAP/TAZ signal in the nucleus and cytoplasm. Specifically, the central plane of each cell is first extracted. DAPI and YAP/TAZ channels are then used to produce masks for cell nucleus and cell body respectively. Nucleus YAP/TAZ is calculated by averaging YAP/TAZ intensity within the nucleus mask and cytoplasmic YAP/TAZ is calculated by averaging YAP/TAZ intensity outside the nucleus mask but within cell body. To note, only YAP/TAZ of focal plane is used for average calculation. For thick collagen bundle size measurement, “Analyze Particle” function from Fiji was used.

### Rheometry

An Anton Parr Shear Stress Rheometer was used for mechanical characterization of collagen gels with the 25-mm parallel-plate geometry and a 500 μm gap. To prevent slip between the gel and the plate, no. 1 25-mm cover glasses (VWR) used for the top plate and a 40 mm cover glass were chemically treated with polydopamine and attached to each plate of the rheometer with double-sided tape (3M 666). The rheometer was then zeroed and calibrated. The temperature of the system is preset and maintained at 37° C. Around 300 μL collagen was pipetted onto the rheometer, the top plate was quickly lowered 500 μm, and the sample was kept in a custom-made humidity chamber to prevent evaporation. After at least 60 minutes of gelation, gels were immersed in PBS and allowed to sit for at least 15 minutes before taking measurements. Then, the shear modulus was measured at 2% strain and at 0.1 Hz. The shear modulus was determined and analyzed using custom Python scripts. Frequency sweeps were carried out at 2% strain.

In the testing of hydrogels, collagen was deposited onto the bottom plate of the rheometer immediately before gelation, and the top plate was lowered rapidly so that the gel formed a uniform disk between the two plates. Approximately, 350 μL collagen was pipetted onto the rheometer, the gap was set to 500 μm, and the sample was kept in a custom made humidity chamber to prevent evaporation. Polymerization progress was monitored by imposing three cycles of 0.5% strain every 5 min, measuring the shear storage modulus G′ as a function of polymerization time. After at least 60 minutes of gelation, gels were immersed in PBS and allowed to sit for at least 15 minutes before taking measurements. For strain sweep measurements, collagen gels were subjected to 5 oscillations at 0.1 Hz at increasing amplitudes from 2 to 12% in 2% increments and 12 to 100% in 4% increments. Custom Python scripts were used to determine the differential shear moduli and subsequent metrics. For stress relaxation measurements, strains were applied with a rise time of 0.15 s. Only one stress relaxation test is conducted on any given sample. Custom Python scripts were used to determine the relaxation moduli, peak stress, and half max relaxation time (t).

### Scanning Electron Microscopy

The collagen sample is placed in 4% PFA for 1 hour at room temperature. The sample is then soaked twice in PBS for 10 minutes each. This is followed by two 10-minute ddH2O washes. The sample is then put through a graded ethanol+H2O wash for 10 minutes each in this order: 30% EtOH, 50% EtOH, 66% EtOH, and 100% EtOH. The sample was then put through a graded ethanol +HMDS wash for 10 minutes each in this order: 30% HMDS, 50% HMDS, 66% HMDS, and 100% HMDS. Samples were then placed on aluminum foil and allowed to dry in the fume hood overnight. Subsequently, the samples were mounted on a support with carbon tape and covered with an 8 nm layer of iridium with a sputter coater. The samples were then imaged with a scanning electron microscope.

For hydrogels, the collagen sample is placed in 4% paraformaldehyde (Thermo Fisher Scientific) for 1 hour at room temperature. The sample is then washed twice in PBS for 10 minutes each. This is followed by two 10-minute ddH2O washes. The sample was then through a graded ethanol+H2O wash for 10 minutes each in this order: 30% EtOH, 50% EtOH, 66%

EtOH, and 100% EtOH. The sample was then put through a graded ethanol+HMDS wash for 10 minutes each in this order: 30% HMDS, 50% HMDS, 66% HMDS, and 100% HMDS. Samples were then placed on aluminum foil and allowed to dry in the fume hood overnight. Subsequently, the samples were mounted on a support with carbon tape and covered with a 8 nm layer of iridium with a sputter coater. The samples were then imaged with a scanning electron microscope.

### Statistical Analysis

Sampling and statistical analyses of various results plots are indicated in their corresponding figure caption. For shear modulus, unpaired t-test is performed. For statistical comparisons in drug studies, one-way ANOVA with post-hoc Tukey HSD test was performed. * indicates p-values<0.05. ** indicates p-value<0.01. *** indicates p-value<0.001, **** indicates p-value<0.0001. # indicates current condition is significantly different (p<0.05) with every other condition unless otherwise annotated.

GraphPad Prism was used for all statistical analyses. In vitro experiments were repeated at least three independent times. To compare differences between more than two groups, a one-way ANOVA with Tukey's post-hoc test was used. Different levels of statistical significance were set at *p<0.05, **p<0.01, ***p<0.001, ****p<0.0001.

### SHG Imaging

HCC cases were selected from the Yale New Haven Hospital pathology database. No approval from a research ethics committee was required for this study, as coded tissue obtained from routine diagnostic workflow was used and the included patients are not affected by the study. Anonymous or coded use of redundant tissue for research purposes is part of the standard treatment agreement with patients, to which patients may opt out. None of the included patients submitted an objection against use of residual material.

Antigen retrieval and slide staining for SHG imaging were adapted from previous protocols. Formalin fixed slides were first incubated at 55° C. for 10 minutes. Slides were then immersed in Xylene baths 2 times, 5 min each time, to wash off paraffin. Slides were then immersed in a series of ethanol baths to rehydrate tissue. Slides were then soaked in DI water to hydrate. Tissues were then immersed in 10 mM Sodium Citrate, 0.05% Tween 20, pH 6 solution at 95° C. for 15 minutes. The solution was then brought down to room temperature, and slides were removed and carefully dried. The sample was then outlined with a hydrophobic pen. The sample was then soaked in TB S+0.025% TritonX-100 two times for 5 minutes each. The tissue was then blocked in TBS+10% BSA at RT for 2 hours. Tissue samples were then stained with DAPI for 1 hour at room temperature. Samples were mounted in Vectashield before imaging.

Images were acquired using two-photon microscopy (MaiTai Ti:Sapphire Laser, Spectra Physics) with a ×40 and excitation at 890 and 1,090 nm.

### COMSOL Simulations

Flow simulations corresponding to the pipetting cycles were performed in Comsol Multiphysics. The computational domain was originally limited to the pipette tip, however due to potentially significant interactions between the flow and the microcentrifuge tube, the domain was expanded to include the fluid both inside the micropipette tip and the microcentrifuge tube. The geometry of the P200 micropipette (Thermo Scientific ART) and the 1.5 mL microcentrifuge tube (USA Scientific) were built in Comsol Multiphysics as an axisymmetric model. The model was meshed using the adaptive mesh refinement tool within the Time-Dependent Solver to obtain a grid of 5716 triangular elements and 438 edge elements. The effects of Col. I crosslinking and other chemical reactions were ignored, so the properties of the fluid domain were simplified as water at 303.15 K. To simulate the cycle of pipetting in and pipetting out at different frequencies, the inlet boundary condition of the Laminar Flow study was defined by overlapping two functions: a positive mass flow rate (m′) times a step function going from 0 to T seconds, and a negative mass flow rate (−m′) times a step function from T to 2T seconds. Periods (T) of 2, 5 and 10 seconds were defined in a Parametric Sweep study. To reduce computational time, a single cycle of flow out-in was simulated for each condition. Thus, the time length of each Time Dependent study was set at 2T (0.1 sec steps) and solved using the PARDISO Direct Solver. Using the solution for shear rate (\dot{γ}), the shear stress (r) was computed for each element through the geometry using for each element through the geometry where μ is the viscosity of the fluid. The computed shear stress was then integrated over the volume of the geometry V to obtain the instantaneous shearing energy for each time step n:

ε[n]=∫ᵥτ[n]dV

which is the average energy between two consecutive timepoints. Therefore, for any length of time T, the total shearing energy delivered to the fluid can be computed as

$$ε [ T ] = {\sum }_{n = 0}^{T} \frac{ε [ n ]}{N}$$

where N is the total number of time steps between times 0 and T.

The disclosures of each and every patent, patent application, and publication cited herein are hereby incorporated herein by reference in their entirety. While this invention has been disclosed with reference to specific embodiments, it is apparent that other embodiments and variations of this invention may be devised by others skilled in the art without departing from the true spirit and scope of the invention. The appended claims are intended to be construed to include all such embodiments and equivalent variations.

What is claimed is:

1. A method of generating thickened collagen bundles or clusters, comprising the steps of:
preparing a neutralized collagen gel sample;
warming the neutralized collagen gel sample to provide a warmed neutralized collagen gel sample; and
agitating the warmed neutralized collagen gel sample to provide an agitated collagen gel sample.

2. The method of claim 1, further comprising the steps of:
cooling the agitated collagen gel sample to about 0° C. for about 10 to 30 minutes to provide a cooled collagen gel sample; and
reagitating the cooled collagen gel sample at a temperature greater than or equal to 0° C.

3. The method of claim 1, further comprising the step of allowing the neutralized collagen gel sample to sit at room temperature for 6 to 10 minutes.

4. The method of claim 1, further comprising the step of cooling the neutralized collagen gel sample to 0° C.

5. The method of claim 1, wherein the step of warming the neutralized collagen gel sample comprises the step of heating the neutralized collagen gel sample to a temperature greater than or equal to 20° C.

6. The method of claim 1, wherein the step of agitating the warmed neutralized collagen gel sample comprises the step of mixing the warmed neutralized collagen gel sample with a pipette.

7. The method of claim 1, wherein the agitated collagen gel sample has a turbidity which measures above background.

8. The method of claim 1, wherein the step of agitating the warmed neutralized gel sample is automated via a mixing device.

9. The method of claim 1, wherein the step of agitating the warmed neutralized collagen gel is performed for over about 10 minutes.

10. The method of claim 9, wherein the step of agitating the warmed neutralized collagen gel provides disconnected collagen clusters.

11. The method of claim 10, further comprising the step of mixing the disconnected collagen clusters with cells to provide a cell and collagen cluster mixture.

12. The method of claim 11, wherein the cell and collagen cluster mixture self-aggregates.

13. The method of claim 11, wherein the cell and collagen cluster mixture comprises a structure selected from the group consisting of compact cell-collagen clusters, spheroids, and fibrotic cell clusters.

14. The method of claim 1, wherein the neutralized collagen sample has a collagen concentration of about 1 mg/mL to about 10 mg/mL.

15. The method of claim 1, wherein the neutralized collagen sample comprises a collagen selected from the group consisting of type I collagen, type II collagen, type III collagen, type IV collagen, type XI collagen, thick collagen, hydrolyzed collagen, mammal collagen, powdered collagen, and synthetic collagen.

16. A thickened collagen structure produced by the method of claim 1.

17. The thickened collagen structure of claim 16, wherein the thickened collagen structure is macroporous or mesoporous.

18. The thickened collagen structure of claim 16, wherein the thickened collagen structure is used in a bioactive cue, an in situ injectable, a vaccine, a biomimetic material, or a vascular organoid.

19. The thickened collagen structure of claim 16, wherein the thickened collagen structure comprises at least one collagen island.

20. The thickened collagen structure of claim 19, wherein the area of the at least one collagen island is between 1 and 1500 μm².
